SEQUENCE LISTING (76 sequences)

SEQ ID NO: 1 - DNA, 183 bp, synthetic construct
  source 1..183: mol_type = other DNA
cgcaagcgctggcagaacgagaagctgggcctggacgccggcgacgagtacgaggacgagaacctgtacgagggcctgaacctggacgactgctccatgtacgaggacatctcccgcggcctgcagggcacctaccaggacgtgggctccctgaacatcggcgacgtgcagctggagaagccc

SEQ ID NO: 2 - DNA, 183 bp, synthetic construct
  source 1..183: mol_type = other DNA
cgcaagcgctggcagaacgagaagctgggcctggacgccggcgacgagtacgaggacgagaacctgtacgagggcctgaacctggacgactgcgccatgtacgaggacatcgcccgcggcctgcagggcgtgtaccaggacgtgggctccctgaacatcggcgacgtgcagctggagaagccc

SEQ ID NO: 3 - DNA, 147 bp, synthetic construct
  source 1..147: mol_type = other DNA
ctggacaaggacgactccaaggccggcatggaggaggaccacacctacgagggcctggacatcgaccagaccgccacctacgaggacatcgtgaccctgcgcaccggcgaggtgaagtggtccgtgggcgagcaccccggccaggag

SEQ ID NO: 4 - DNA, 360 bp, synthetic construct
  source 1..360: mol_type = other DNA
tggcgccgcaagcgcaaggagaagcagtccgagacctcccccaaggagttcctgaccatctacgaggacgtgaaggacctgaagacccgccgcaaccacgagcaggagcagaccttccccggcggcggctccaccatctactccatgatccagtcccagtcctccgcccccacctcccaggagcccgcctacaccctgtactccctgatccagccctcccgcaagtccggctcccgcaagcgcaaccactccccctccttcaactccaccatctacgaggtgatcggcaagtcccagcccaaggcccagaaccccgcccgcctgtcccgcaaggagctggagaacttcgacgtgtactcc

SEQ ID NO: 5 - DNA, 72 bp, synthetic construct
  source 1..72: mol_type = other DNA
ctgtgcgcccgcccccgccgctcccccgcccaggaggacggcaaggtgtacatcaacatgcccggccgcggc

SEQ ID NO: 6 - DNA, 267 bp, synthetic construct
  source 1..267: mol_type = other DNA
ttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 7 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 8 - DNA, 182 bp, synthetic construct
  source 1..182: mol_type = other DNA
aaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaag

SEQ ID NO: 9 - DNA, 512 bp, synthetic construct
  source 1..512: mol_type = other DNA
tttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattag

SEQ ID NO: 10 - DNA, 242 bp, synthetic construct
  source 1..242: mol_type = other DNA
attccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaa

SEQ ID NO: 11 - DNA, 167 bp, synthetic construct
  source 1..167: mol_type = other DNA
agtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaag

SEQ ID NO: 12 - DNA, 351 bp, synthetic construct
  source 1..351: mol_type = other DNA
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 13 - DNA, 210 bp, synthetic construct
  source 1..210: mol_type = other DNA
atccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 14 - DNA, 144 bp, synthetic construct
  source 1..144: mol_type = other DNA
aagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 15 - DNA, 141 bp, synthetic construct
  source 1..141: mol_type = other DNA
aaggctctgatccacctgagcgacctccgggagtacaggcgctttgagaaggagaagctcaagtcccagtggaacaatgataatccccttttcaagagcgccaccacgacggtcatgaaccccaagtttgctgagagttag

SEQ ID NO: 16 - DNA, 144 bp, synthetic construct
  source 1..144: mol_type = other DNA
aaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 17 - DNA, 861 bp, synthetic construct
  source 1..861: mol_type = other DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 18 - DNA, 573 bp, synthetic construct
  source 1..573: mol_type = other DNA
tataaagttgacttggttctgttctataggcgcatagcggaaagagacgagacactaacagatggtaaaacatatgatgcctttgtgtcttacctgaaagagtgtcatcctgagaataaagaagagtatacttttgctgtggagacgttacccagggtcctggagaaacagtttgggtataagttatgcatatttgaaagagatgtggtgcctggcggagctgttgtcgaggagatccattcactgatagagaaaagccggaggctaatcatcgttctcagccagagttacctgactaacggagccaggcgtgagctcgagagtggactccacgaagcactggtagagaggaagattaagatcatcttaattgagtttactccagccagcaacatcacctttctccccccgtcgctgaaactcctgaagtcctacagagttctaaaatggagggctgacagtccctccatgaactcaaggttctggaagaatcttgtttacctgatgcccgcaaaagccgtcaagccatggagagaggagtcggaggcgcggtctgttctctcagcaccttga

SEQ ID NO: 19 - DNA, 279 bp, synthetic construct
  source 1..279: mol_type = other DNA
aacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtga

SEQ ID NO: 20 - DNA, 660 bp, synthetic construct
  source 1..660: mol_type = other DNA
cattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 21 - DNA, 858 bp, synthetic construct
  source 1..858: mol_type = other DNA
agcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 22 - DNA, 1542 bp, synthetic construct
  source 1..1542: mol_type = other DNA
cccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctga

SEQ ID NO: 23 - DNA, 375 bp, synthetic construct
  source 1..375: mol_type = other DNA
atggccttaccagtgaccgccttgctcctgccgctggccttgctgctccacgccgccaggccggacggcaacgaggagatgggcggcatcacccagaccccctacaaggtgtccatctccggcaccaccgtgatcctgacctgcccccagtaccccggctccgagatcctgtggcagcacaacgacaagaacatcggcggcgacgaggacgacaagaacatcggctccgacgaggaccacctgtccctgaaggagttctccgagctggagcagtccggctactacgtgtgctacccccgcggctccaagcccgaggacgccaacttctacctgtacctgcgcgcccgcgtgtgcgagaactgcatggagatggac

SEQ ID NO: 24 - DNA, 468 bp, synthetic construct
  source 1..468: mol_type = other DNA
atggccttaccagtgaccgccttgctcctgccgctggccttgctgctccacgccgccaggccgaacaagatcctggtgaagcagtcccccatgctggtggcctacgacaacgccgtgaacctgtcctgcaagtactcctacaacctgttctcccgcgagttccgcgcctccctgcacaagggcctggactccgccgtggaggtgtgcgtggtgtacggcaactactcccagcagctgcaggtgtactccaagaccggcttcaactgcgacggcaagctgggcaacgagtccgtgaccttctacctgcagaacctgtacgtgaaccagaccgacatctacttctgcaagatcgaggtgatgtacccccccccctacctggacaacgagaagtccaacggcaccatcatccacgtgaagggcaagcacctgtgcccctcccccctgttccccggcccctccaagcccttc

SEQ ID NO: 25 - DNA, 795 bp, synthetic construct
  source 1..795: mol_type = other DNA
atggccttaccagtgaccgccttgctcctgccgctggccttgctgctccacgccgccaggccgggatcccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggtggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaagctagc

SEQ ID NO: 26 - DNA, 864 bp, synthetic construct
  source 1..864: mol_type = other DNA
atggccttaccagtgaccgccttgctcctgccgctggccttgctgctccacgccgccaggccggacatccagatgacacaaaccacctccagcctgtccgcatcccttggagatcgagtcaccattagctgccgggcgtcccaggacatcagcaaatacctcaattggtatcagcaaaaacccgacggcacggtgaagcttctgatataccacacttcacggctgcactcaggcgtgccaagtcggttttcaggatccggctccggcaccgattattctctgaccattagcaatctcgagcaagaggatatcgccacttatttctgtcagcaggggaacaccctcccttatacgtttggtggtgggaccaagttggagattacacgggcagatgccgcccctactgtgagcatatttcctcctagctctaatgggggtggaggctccggtggtggggggagcggcggcggtggctcaggaggcggcggcagcgaggtcaaactgcaagagtctggtccaggcctggtggccccctcccagtctctcagtgtgacctgtaccgtttcaggcgtgtctttgcctgattacggcgtgtcctggataaggcagccaccaagaaagggactggaatggctgggcgttatctggggatcagagacaacttattacaactcagctcttaaatccagactcacgatcattaaggataactctaaatcccaggtgttcctgaagatgaattctctccagacagacgatactgccatctactattgcgctaagcattattattacgggggctcatacgccatggactactggggacagggcaccagcgtgactgtgagttcccctagggctagc

SEQ ID NO: 27 - DNA, 798 bp, synthetic construct
  source 1..798: mol_type = other DNA
atggccctgcccgtgaccgctctcctgctgcctctggccctgctcctccatgctgccagacccgacatccagatgacacagacaaccagcagcctgtccgcttccctcggagacagggtgacaatttcctgcagggccagccaggacatcagcaagtacctgaactggtaccagcagaaacccgacggcaccgtcaagctcctgatctaccacaccagcagactgcacagcggagtgccttccaggttcagcggcagcggctccggcaccgattactccctgaccattagcaacttagaacaggaggacattgccacctacttttgtcagcagggcaacaccctcccctacacctttggaggcggaaccaagttagaaatcaccggcggcggcggcagcggaggaggaggcagcggaggcggaggctccgaggtgaaactgcaggagagcggccccggactggtcgcccctagccaatccctctccgtcacctgcaccgtgagcggagtgagcctgcctgactacggagtgagctggatcagacagccccctaggaaaggactggaatggctgggcgtgatttggggcagcgagaccacctattacaacagcgccctgaagtccagactgacaatcatcaaggacaatagcaaaagccaagtgtttctgaagatgaacagcctgcagaccgatgacaccgccatctattattgcgccaagcactactactacggaggaagctacgctatggattattggggccaaggcacaagcgtgaccgtcagcagcgcggccgcc

SEQ ID NO: 28 - DNA, 921 bp, synthetic construct
  source 1..921: mol_type = other DNA
atggccttaccagtgaccgccttgctcctgccgctggccttgctgctccacgccgccaggccgatggaaaaggatacactgttgttgtgggttctgctcctgtgggtgcccggcagcaccggagatattgtgctgacgcagtctcctgcatcactcgccgtgtctctgggccagcgcgctaccatcagctgcagagcctctgaaagtgttgacaattatggaatttctttcatgaattggttccagcagaagcctggccagcccccgaaactcctcatatatgccgcgtctaatcagggctctggggtccctgctagattttctggcagcggctccggcaccgacttcagtctgaatatacatcccatggaagaagacgataccgccatgtacttttgccaacaatctaaggaggtgccttggacgttcggcggcggtacgaagctggaaattaagggcggcgggggaagcggcggggggggatcaggcgggggtggctccggaggcggtggaagtatgggctggagttggatcttccttttccttctttctggtaccgcgggagtgcactctgaggtgcagctccagcagtccggccccgagctcgtcaagcctggggccagtgtcaagatttcctgtaaggcatctggatatacctttacagattacaatatgcattgggtgaaacagtcacatggaaagtcactcgagtggatcggatacatttacccttacaatggaggaaccggatataatcagaagtttaagagcaaggccacactcacggtggacaattcttcatctacagcctacatggatgttcggtctctgacttccgaggatagtgcggtgtattactgcgccaggggacgccccgctatggattactgggggcagggaacctctgtaacagttagctca

SEQ ID NO: 29 - DNA, 1044 bp, synthetic construct
  source 1..1044: mol_type = other DNA
atggccttaccagtgaccgccttgctcctgccgctggccttgctgctccacgccgccaggccggacttcgtgatgactcagtctcctagctccctgaccgtgacagccggcgagaaggtgaccatgtcctgcaaatctagtcagagtctgctgaactcaggcaatcagaagaactatctgacatggtacctgcagaagccagggcagccccctaaactgctgatctattgggccagcaccagggaatccggcgtgcccgacagattcaccggctccgggtctggaacagattttactctgaccatttcaagcgtgcaggccgaggacctggctgtgtactattgtcagaatgattacagctatccctacacatttggcgggggaactaagctggaaatcaaaggtggtggtggttctggtggtggtggttccggcggcggcggctccggtggtggtggatccgaggtgcagctgcagcagagtggacccgaactggtgaaacctggcgcctccgtgaaaatgtcttgcaaggctagtgggtacaccttcacagactactatatgaaatgggtgaagcagtcacacgggaagagcctggagtggatcggagatatcattccctctaacggcgccactttctacaatcagaagtttaaaggcaaggctactctgaccgtggaccggagctcctctaccgcctatatgcacctgaacagtctgacatcagaagatagcgctgtgtactattgtacacggtcccatctgctgagagcctcttggtttgcttattggggccaggggacactggtgactgtgagctccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtg

SEQ ID NO: 30 - DNA, 639 bp, synthetic construct
  source 1..639: mol_type = other DNA
atggccttaccagtgaccgccttgctcctgccgctggccttgctgctccacgccgccaggccgggcatgcgcaccgaggacctgcccaaggccgtggtgttcctggagccccagtggtaccgcgtgctggagaaggactccgtgaccctgaagtgccagggcgcctactcccccgaggacaactccacccagtggttccacaacgagtccctgatctcctcccaggcctcctcctacttcatcgacgccgccaccgtggacgactccggcgagtaccgctgccagaccaacctgtccaccctgtccgaccccgtgcagctggaggtgcacatcggctggctgctgctgcaggccccccgctgggtgttcaaggaggaggaccccatccacctgcgctgccactcctggaagaacaccgccctgcacaaggtgacctacctgcagaacggcaagggccgcaagtacttccaccacaactccgacttctacatccccaaggccaccctgaaggactccggctcctacttctgccgcggcctgttcggctccaagaacgtgtcctccgagaccgtgaacatcaccatcacccagggcctggccgtgcccaccatctcctccttcttcccccccggctaccag

SEQ ID NO: 31 - DNA, 615 bp, synthetic construct
  source 1..615: mol_type = other DNA
atggccttaccagtgaccgccttgctcctgccgctggccttgctgctccacgccgccaggccgcaggccgccgccccccccaaggccgtgctgaagctggagcccccctggatcaacgtgctgcaggaggactccgtgaccctgacctgccagggcgcccgctcccccgagtccgactccatccagtggttccacaacggcaacctgatccccacccacacccagccctcctaccgcttcaaggccaacaacaacgactccggcgagtacacctgccagaccggccagacctccctgtccgaccccgtgcacctgaccgtgctgtccgagtggctggtgctgcagaccccccacctggagttccaggagggcgagaccatcatgctgcgctgccactcctggaaggacaagcccctggtgaaggtgaccttcttccagaacggcaagtcccagaagttctcccacctggaccccaccttctccatcccccaggccaaccactcccactccggcgactaccactgcaccggcaacatcggctacaccctgttctcctccaagcccgtgaccatcaccgtgcaggtgccctccatgggctcctcctcccccatgggc

SEQ ID NO: 32 - DNA, 894 bp, synthetic construct
  source 1..894: mol_type = other DNA
atggccttaccagtgaccgccttgctcctgccgctggccttgctgctccacgccgccaggccgcaggtggacaccaccaaggccgtgatcaccctgcagcccccctgggtgtccgtgttccaggaggagaccgtgaccctgcactgcgaggtgctgcacctgcccggctcctcctccacccagtggttcctgaacggcaccgccacccagacctccaccccctcctaccgcatcacctccgcctccgtgaacgactccggcgagtaccgctgccagcgcggcctgtccggccgctccgaccccatccagctggagatccaccgcggctggctgctgctgcaggtgtcctcccgcgtgttcaccgagggcgagcccctggccctgcgctgccacgcctggaaggacaagctggtgtacaacgtgctgtactaccgcaacggcaaggccttcaagttcttccactggaactccaacctgaccatcctgaagaccaacatctcccacaacggcacctaccactgctccggcatgggcaagcaccgctacacctccgccggcatctccgtgaccgtgaaggagctgttccccgcccccgtgctgaacgcctccgtgacctcccccctgctggagggcaacctggtgaccctgtcctgcgagaccaagctgctgctgcagcgccccggcctgcagctgtacttctccttctacatgggctccaagaccctgcgcggccgcaacacctcctccgagtaccagatcctgaccgcccgccgcgaggactccggcctgtactggtgcgaggccgccaccgaggacggcaacgtgctgaagcgctcccccgagctggagctgcaggtgctgggcctgcagctgcccacccccgtgtggttccac

SEQ ID NO: 33 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 34 - DNA, 252 bp, synthetic construct
  source 1..252: mol_type = other DNA
tccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 35 - DNA, 72 bp, synthetic construct
  source 1..72: mol_type = other DNA
ccattttttttctgctgcttcatcgctgtagccatgggaatccgtttcattattatggtaacaatatggagt

SEQ ID NO: 36 - DNA, 252 bp, synthetic construct
  source 1..252: mol_type = other DNA
cacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 37 - DNA, 73 bp, synthetic construct
  source 1..73: mol_type = other DNA
gtctctttctgcttggtgatggtactcctttttgcagtggacacaggactatatttctctgtgaagacaaaca

SEQ ID NO: 38 - DNA, 252 bp, synthetic construct
  source 1..252: mol_type = other DNA
tgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgcc

SEQ ID NO: 39 - DNA, 72 bp, synthetic construct
  source 1..72: mol_type = other DNA
ctggtgcctgtgttctgtggactcctcgtagccaagagcctggtgctgtcagccctgctcgtctggtggggg

SEQ ID NO: 40 - DNA, 252 bp, synthetic construct
  source 1..252: mol_type = other DNA
tccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 41 - DNA, 72 bp, synthetic construct
  source 1..72: mol_type = other DNA
gctggtacagtcctcctccttcgggctggattctatgctgtcagctttctctctgtggccgtgggcagcacc

SEQ ID NO: 42 - DNA, 252 bp, synthetic construct
  source 1..252: mol_type = other DNA
ttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcgg

SEQ ID NO: 43 - DNA, 72 bp, synthetic construct
  source 1..72: mol_type = other DNA
atgggcctggcctttctagtcctggtggctctagtgtggttcctggttgaagactggctcagcaggaagagg

SEQ ID NO: 44 - DNA, 252 bp, synthetic construct
  source 1..252: mol_type = other DNA
agggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacat

SEQ ID NO: 45 - DNA, 69 bp, synthetic construct
  source 1..69: mol_type = other DNA
gttctgattgggacctcagtggtcaaaatccctttcaccatcctcctcttctttctccttcatcgctgg

SEQ ID NO: 46 - DNA, 210 bp, synthetic construct
  source 1..210: mol_type = other DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 47 - DNA, 78 bp, synthetic construct
  source 1..78: mol_type = other DNA
ctcactgccgaggtcctaggaatcatttgcattgtcctgatggccactgtgttaaaaacaatagttcttattcctttc

SEQ ID NO: 48 - DNA, 252 bp, synthetic construct
  source 1..252: mol_type = other DNA
gtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 49 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 50 - DNA, 252 bp, synthetic construct
  source 1..252: mol_type = other DNA
gcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacacc

SEQ ID NO: 51 - DNA, 84 bp, synthetic construct
  source 1..84: mol_type = other DNA
attccgtggctcggccacctcctcgtgggcctcagcggggcttttggcttcatcatcttagtgtacttgctgatcaactgcagg

SEQ ID NO: 52 - protein, 15 aa, synthetic construct
GGGGSGGGGSGGGGS

SEQ ID NO: 53 - protein, 20 aa, synthetic construct
GGGGSGGGGSGGGGSGGGGS

SEQ ID NO: 54 - protein, 22 aa, synthetic construct
GSGATNFSLLKQAGDVEENPGP

SEQ ID NO: 55 - protein, 465 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 56 - protein, 152 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWS

SEQ ID NO: 57 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 137 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIA

SEQ ID NO: 59 - protein, 42 aa, synthetic construct
KRGRKKLLYIFKQPFMRPVQTTQEEDGCSCRFPEEEEGGCEL

SEQ ID NO: 60 - protein, 264 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 286 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 62 - protein, 423 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIA

SEQ ID NO: 63 - protein, 179 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 64 - DNA, 858 bp, synthetic construct
  source 1..858: mol_type = other DNA
aactgccgcaacaccggcccctggctgaagaaggtgctgaagtgcaacacccccgacccctccaagttcttctcccagctgtcctccgagcacggcggcgacgtgcagaagtggctgtcctcccccttcccctcctcctccttctcccccggcggcctggcccccgagatctcccccctggaggtgctggagcgcgacaaggtgacccagctgctgctgcagcaggacaaggtgcccgagcccgcctccctgtcctccaaccactccctgacctcctgcttcaccaaccagggctacttcttcttccacctgcccgacgccctggagatcgaggcctgccaggtgtacttcacctacgacccctactccgaggaggaccccgacgagggcgtggccggcgcccccaccggctcctccccccagcccctgcagcccctgtccggcgaggacgacgcctactgcaccttcccctcccgcgacgacctgctgctgttctccccctccctgctgggcggcccctcccccccctccaccgcccccggcggctccggcgccggcgaggagcgcatgcccccctccctgcaggagcgcgtgccccgcgactgggacccccagcccctgggcccccccacccccggcgtgcccgacctggtggacttccagcccccccccgagctggtgctgcgcgaggccggcgaggaggtgcccgacgccggcccccgcgagggcgtgtccttcccctggtcccgcccccccggccagggcgagttccgcgccctgaacgcccgcctgcccctgaacaccgacgcctacctgtccctgcaggagctgcagggccaggaccccacccacctggtg

SEQ ID NO: 65 - DNA, 348 bp, synthetic construct
  source 1..348: mol_type = other DNA
atggagcagggcaagggcctggccgtgctgatcctggccatcatcctgctgcagggcaccctggcccagtccatcaagggcaaccacctggtgaaggtgtacgactaccaggaggacggctccgtgctgctgacctgcgacgccgaggccaagaacatcacctggttcaaggacggcaagatgatcggcttcctgaccgaggacaagaagaagtggaacctgggctccaacgccaaggacccccgcggcatgtaccagtgcaagggctcccagaacaagtccaagcccctgcaggtgtactaccgcatgtgccagaactgcatcgagctgaacgccgccaccatctcc

SEQ ID NO: 66 - DNA, 315 bp, synthetic construct
  source 1..315: mol_type = other DNA
atggagcactccaccttcctgtccggcctggtgctggccaccctgctgtcccaggtgtcccccttcaagatccccatcgaggagctggaggaccgcgtgttcgtgaactgcaacacctccatcacctgggtggagggcaccgtgggcaccctgctgtccgacatcacccgcctggacctgggcaagcgcatcctggacccccgcggcatctaccgctgcaacggcaccgacatctacaaggacaaggagtccaccgtgcaggtgcactaccgcatgtgccagtcctgcgtggagctggaccccgccaccgtggcc

SEQ ID NO: 67 - DNA, 78 bp, synthetic construct
  source 1..78: mol_type = other DNA
gtgatgtccgtggccaccatcgtgatcgtggacatctgcatcaccggcggcctgctgctgctggtgtactactggtcc

SEQ ID NO: 68 - DNA, 63 bp, synthetic construct
  source 1..63: mol_type = other DNA
ggcttcctgttcgccgagatcgtgtccatcttcgtgctggccgtgggcgtgtacttcatcgcc

SEQ ID NO: 69 - DNA, 63 bp, synthetic construct
  source 1..63: mol_type = other DNA
ggcatcatcgtgaccgacgtgatcgccaccctgctgctggccctgggcgtgttctgcttcgcc

SEQ ID NO: 70 - protein, 541 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRKRWQNEKLGLDAGDEYEDENLYEGLNLDQTATYEDISRGLQGTYQDVGSLNIGDVQLEKPWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGSGATNFSLLKQAGDVEENPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIALDKDDSKAGMEEDHTYEGLDIDQTATYEDIVTLRTGEVKWSVGEHPGQE

SEQ ID NO: 71 - protein, 916 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRKRWQNEKLGLDAGDEYEDENLYEGLNLDQTATYEDISRGLQGTYQDVGSLNIGDVQLEKPFWLERTMPRIPTLKNLEDLVTEYHGNFSAWSGVSKGLAESLQPDYSERLCLVSEIPPKGGALGEGPGASPCNQHSPYWAPPCYTLKPETWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGSGATNFSLLKQAGDVEENPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIALDKDDSKAGMEEDHTYEGLDIDQTATYEDIVTLRTGEVKWSVGEHPGQENCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 72 - protein, 840 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 73 - DNA, 948 bp, synthetic construct
  source 1..948: mol_type = other DNA
atgcagtccggcacccactggcgcgtgctgggcctgtgcctgctgtccgtgggcgtgtggggccaggacggcaacgaggagatgggcggcatcacccagaccccctacaaggtgtccatctccggcaccaccgtgatcctgacctgcccccagtaccccggctccgagatcctgtggcagcacaacgacaagaacatcggcggcgacgaggacgacaagaacatcggctccgacgaggaccacctgtccctgaaggagttctccgagctggagcagtccggctactacgtgtgctacccccgcggctccaagcccgaggacgccaacttctacctgtacctgcgcgcccgcgtgtgcgagaactgcatggagatggacgtgatgtccgtggccaccatcgtgatcgtggacatctgcatcaccggcggcctgctgctgctggtgtactactggtccaagaacggaagcggagccacgaacttctctctgttaaagcaagcaggagatgttgaagaaaaccccgggcctatggagcagggcaagggcctggccgtgctgatcctggccatcatcctgctgcagggcaccctggcccagtccatcaagggcaaccacctggtgaaggtgtacgactaccaggaggacggctccgtgctgctgacctgcgacgccgaggccaagaacatcacctggttcaaggacggcaagatgatcggcttcctgaccgaggacaagaagaagtggaacctgggctccaacgccaaggacccccgcggcatgtaccagtgcaagggctcccagaacaagtccaagcccctgcaggtgtactaccgcatgtgccagaactgcatcgagctgaacgccgccaccatctccggcttcctgttcgccgagatcgtgtccatcttcgtgctggccgtgggcgtgtacttcatcgccggctaataa

SEQ ID NO: 74 - DNA, 1785 bp, synthetic construct
  source 1..1785: mol_type = other DNA
atgcagtccggcacccactggcgcgtgctgggcctgtgcctgctgtccgtgggcgtgtggggccaggacggcaacgaggagatgggcggcatcacccagaccccctacaaggtgtccatctccggcaccaccgtgatcctgacctgcccccagtaccccggctccgagatcctgtggcagcacaacgacaagaacatcggcggcgacgaggacgacaagaacatcggctccgacgaggaccacctgtccctgaaggagttctccgagctggagcagtccggctactacgtgtgctacccccgcggctccaagcccgaggacgccaacttctacctgtacctgcgcgcccgcgtgtgcgagaactgcatggagatggacgtgatgtccgtggccaccatcgtgatcgtggacatctgcatcaccggcggcctgctgctgctggtgtactactggtccggcagtggctggcgccgcaagcgcaaggagaagcagtccgagacctcccccaaggagttcctgaccatctacgaggacgtgaaggacctgaagacccgccgcaaccacgagcaggagcagaccttccccggcggcggctccaccatctactccatgatccagtcccagtcctccgcccccacctcccaggagcccgcctacaccctgtactccctgatccagccctcccgcaagtccggctcccgcaagcgcaaccactccccctccttcaactccaccatctacgaggtgatcggcaagtcccagcccaaggcccagaaccccgcccgcctgtcccgcaaggagctggagaacttcgacgtgtactccggcagtggccgcgtgaagttctcccgctccgccgacgcccccgcctacaagcagggccagaaccagctgtacaacgagctgaacctgggccgccgcgaggagtacgacgtgctggacaagcgccgcggccgcgaccccgagatgggcggcaagccccgccgcaagaacccccaggagggcctgtacaacgagctgcagaaggacaagatggccgaggcctactccgagatcggcatgaagggcgagcgccgccgcggcaagggccacgacggcctgtaccagggcctgtccaccgccaccaaggacacctacgacgccctgcacatgcaggccctgcccccccgcggaagcggagccacgaacttctctctgttaaagcaagcaggagatgttgaagaaaaccccgggcctatggagcagggcaagggcctggccgtgctgatcctggccatcatcctgctgcagggcaccctggcccagtccatcaagggcaaccacctggtgaaggtgtacgactaccaggaggacggctccgtgctgctgacctgcgacgccgaggccaagaacatcacctggttcaaggacggcaagatgatcggcttcctgaccgaggacaagaagaagtggaacctgggctccaacgccaaggacccccgcggcatgtaccagtgcaagggctcccagaacaagtccaagcccctgcaggtgtactaccgcatgtgccagaactgcatcgagctgaacgccgccaccatctccggcttcctgttcgccgagatcgtgtccatcttcgtgctggccgtgggcgtgtacttcatcgccggctccggcgccctgtacctgctgcgccgcgaccagcgcctgccccccgacgcccacaagccccccggcggcggctccttccgcacccccatccaggaggagcaggccgacgcccactccaccctggccaagatctaa

SEQ ID NO: 75 - DNA, 2199 bp, synthetic construct
  source 1..2199: mol_type = other DNA
atgcagtccggcacccactggcgcgtgctgggcctgtgcctgctgtccgtgggcgtgtggggccaggacggcaacgaggagatgggcggcatcacccagaccccctacaaggtgtccatctccggcaccaccgtgatcctgacctgcccccagtaccccggctccgagatcctgtggcagcacaacgacaagaacatcggcggcgacgaggacgacaagaacatcggctccgacgaggaccacctgtccctgaaggagttctccgagctggagcagtccggctactacgtgtgctacccccgcggctccaagcccgaggacgccaacttctacctgtacctgcgcgcccgcgtgtgcgagaactgcatggagatggacgtgatgtccgtggccaccatcgtgatcgtggacatctgcatcaccggcggcctgctgctgctggtgtactactggtcccgcgtgaagttctcccgctccgccgacgcccccgcctacaagcagggccagaaccagctgtacaacgagctgaacctgggccgccgcgaggagtacgacgtgctggacaagcgccgcggccgcgaccccgagatgggcggcaagccccgccgcaagaacccccaggagggcctgtacaacgagctgcagaaggacaagatggccgaggcctactccgagatcggcatgaagggcgagcgccgccgcggcaagggccacgacggcctgtaccagggcctgtccaccgccaccaaggacacctacgacgccctgcacatgcaggccctgcccccccgcggaagcggagccacgaacttctctctgttaaagcaagcaggagatgttgaagaaaaccccgggcctatggagcagggcaagggcctggccgtgctgatcctggccatcatcctgctgcagggcaccctggcccagtccatcaagggcaaccacctggtgaaggtgtacgactaccaggaggacggctccgtgctgctgacctgcgacgccgaggccaagaacatcacctggttcaaggacggcaagatgatcggcttcctgaccgaggacaagaagaagtggaacctgggctccaacgccaaggacccccgcggcatgtaccagtgcaagggctcccagaacaagtccaagcccctgcaggtgtactaccgcatgtgccagaactgcatcgagctgaacgccgccaccatctccggcttcctgttcgccgagatcgtgtccatcttcgtgctggccgtgggcgtgtacttcatcgccaagcgcggccgcaagaagctgctgtacatcttcaagcagcccttcatgcgccccgtgcagaccacccaggaggaggacggctgctcctgccgcttccccgaggaggaggagggcggctgcgagctgggaagcggagagggcaggggaagtcttctaacatgcggggacgtggaggaaaatcccggccccatggagcactccaccttcctgtccggcctggtgctggccaccctgctgtcccaggtgtcccccttcaagatccccatcgaggagctggaggaccgcgtgttcgtgaactgcaacacctccatcacctgggtggagggcaccgtgggcaccctgctgtccgacatcacccgcctggacctgggcaagcgcatcctggacccccgcggcatctaccgctgcaacggcaccgacatctacaaggacaaggagtccaccgtgcaggtgcactaccgcatgtgccagtcctgcgtggagctggaccccgccaccgtggccggcatcatcgtgaccgacgtgatcgccaccctgctgctggccctgggcgtgttctgcttcgcctggcgccgcaagcgcaaggagaagcagtccgagacctcccccaaggagttcctgaccatctacgaggacgtgaaggacctgaagacccgccgcaaccacgagcaggagcagaccttccccggcggcggctccaccatctactccatgatccagtcccagtcctccgcccccacctcccaggagcccgcctacaccctgtactccctgatccagccctcccgcaagtccggctcccgcaagcgcaaccactccccctccttcaactccaccatctacgaggtgatcggcaagtcccagcccaaggcccagaaccccgcccgcctgtcccgcaaggagctggagaacttcgacgtgtactcctaa

SEQ ID NO: 76 - DNA, 126 bp, synthetic construct
  source 1..126: mol_type = other DNA
gccctgtacctgctgcgccgcgaccagcgcctgccccccgacgcccacaagccccccggcggcggctccttccgcacccccatccaggaggagcaggccgacgcccactccaccctggccaagatc

(19) United States
(12) Patent Application Publication
(10) Pub. No.: US 2024/0173407 A1
(43) Pub. Date: May 30, 2024

(54) GENERATION OF CD3 EXPRESSING IMMUNE CELLS FOR USE IN CONJUNCTION WITH CD3 BINDING BISPECIFIC TARGETING AGENTS

(71) Applicant: Wugen, Inc., Saint Louis, MO (US)

(72) Inventors: You ZHOU, Creve Coeur, MO (US); Elizabeth SCHRAMM, O'Fallon, IL (US); Matthew COOPER, St. Louis, MO (US)

(21) Appl. No.: 18/501,908

(22) Filed: Nov. 3, 2023

Related U.S. Application Data
(60) Provisional application No. 63/424,120, filed on Nov. 9, 2022.
(60) Provisional application No. 63/422,919, filed on Nov. 4, 2022.

(51) Int. Cl. A61K 39/00; C07K 14/705; C07K 14/725; C07K 16/28; C12N 5/0783

(52) U.S. Cl. CPC A61K 39/4613; A61K 39/4631; A61K 39/4633; A61K 39/464411; C07K 14/70503; C07K 14/7051; C07K 14/70578; C07K 16/2809; C12N 5/0646; A61K 2239/15; C07K 2319/02; C07K 2319/03; C12N 2510/00

(57) ABSTRACT

The present disclosure provides chimeric receptor constructs comprising a CD3e extracellular domain and engineered NK and T cells expressing such receptors and methods of making and using same for the treatment of cancer and other diseases of the immune system.

### CROSS REFERENCE TO RELATED APPLICATIONS

This application claims priority to U.S. Provisional Application Ser. No. 63/422,919, filed on Nov. 4, 2022, and U.S. Provisional Application Ser. No. 63/424,120, filed on Nov. 9, 2022, both of which are incorporated by reference herein in their entirety for all purposes.

### SEQUENCE LISTING

The Sequence Listing XML associated with this application is provided in XML file format and is hereby incorporated by reference into the specification. The name of the XML file containing the Sequence Listing XML is WUGE_004_02US_ST26.xml. The XML file is 96,342 bytes in size, was created on Nov. 3, 2023, and is being submitted electronically via USTPO Patent Center.

### FIELD OF THE INVENTION

The present disclosure relates generally to genetically modified immune cells containing chimeric receptors and methods of making and using the same.

### BACKGROUND

Natural killer (NK) cells are an immune cell type being used to develop cellular therapeutics for cancer treatment. NK cells have properties that make them attractive for therapeutic use, including low toxicity profile and broad tumor killing ability. However, some tumor cells are resistant to endogenous NK cell killing. To circumvent this problem, NK cells can be administered in conjunction with targeting molecules that link the NK cell and the tumor cells.

T cell engagers are engineered proteins designed to crosslink target cells (i.e. Tumor cells) to effector cells (i.e. T cells), triggering cytotoxic killing of the target by the effector. Structurally, bispecific engagers such as bispecific T cell engagers (BiTEs) are typically composed of two distinct single chain variable fragments (scFvs) connected by a protein linker. There are a large number of engagers that harness T cells, including but not limited to BiTEs, trispecific T cell engagers (TriTEs), and quadrispecific T cell engagers, approved for clinical use or in development. However, relatively few engagers that harness NK cells are in development. CD3 is a protein complex that is co-expressed with the T cell receptor. CD3 is composed of the epsilon, delta, gamma and zeta chains. CD3 epsilon is a T cell antigen typically targeted by currently available BiTEs. The engagement of BiTEs to CD3 with a T cell triggers signaling via endogenous immunoreceptor tyrosine-based activation motifs (ITAMs) contained within the TCR:CD3 complex. However, it is generally understood that CD3 epsilon will not traffic to the cell membrane on its own, and that all components of the TCR complex (TCR+CD3 subunits) are required for cell surface trafficking. Additionally, it has been demonstrated CD3 epsilon needs to co-localize with the rest of the TCR:CD3 complex in order for the BiTE:CD3 epsilon interaction to occur. Accordingly, it is generally understood that the TCR complex is a necessary component in the BiTE:CD3 epsilon interaction.

However, the naturally occurring NK cell does not express a TCR:CD3 complex, which presents a challenge in using CD3-targeting BiTEs to engage NK cells. To overcome this challenge, there is a need for engineered NK cells that express receptors capable of interacting with BiTEs or other engager molecules, in order to specifically target NK cells to cancer cells.

### SUMMARY

The disclosure provides an engineered natural killer cell (NK cell) comprising a chimeric receptor comprising a CD3 epsilon (CD3e) extracellular domain, a first transmembrane domain, and a first intracellular domain; wherein the first transmembrane domain does not comprise a CD3e transmembrane domain.

In addition, the disclosure provides an engineered natural killer cell (NK cell) comprising a chimeric receptor comprising a CD3 epsilon (CD3e) extracellular domain, a first transmembrane domain, and a first intracellular domain; wherein the first intracellular domain does not comprise a CD3e intracellular domain.

In some embodiments of the engineered NK cell of the disclosure, the CD3e extracellular domain is noncovalently associated with a CD3 gamma (CD3g) extracellular domain or a CD3 delta (CD3d) extracellular domain. In some embodiments of the engineered NK cell of the disclosure, the CD3e extracellular domain is linked to a CD3 gamma (CD3g) extracellular domain or a CD3 delta (CD3d) extracellular domain by a linker.

In some embodiments of the engineered NK cell of the disclosure, the linker comprises a polypeptide chain comprising a Gly Ser linker.

In some embodiments of the engineered NK cell of the disclosure, the linker comprises the polypeptide sequence set forth in SEQ ID NO. 52 or SEQ ID NO. 53.

In some embodiments of the engineered NK cell of the disclosure, the chimeric receptor is noncovalently associated with a second chimeric receptor.

In some embodiments of the engineered NK cell of the disclosure, the chimeric receptor is linked to a second chimeric receptor by a second linker. In some embodiments, the intracellular domain of the first chimeric receptor is linked to the extracellular domain of the second chimeric receptor by the second linker. In some embodiments, the second linker is a cleavable linker. In some embodiments, the second linker comprises SEQ ID NO. 54.

In some embodiments of the engineered NK cell of the disclosure, the CD3e extracellular domain is capable of binding to a bispecific T cell engager (BiTE).

In some embodiments of the engineered NK cell of the disclosure, the BiTE is Blinatumomab, Tebentafusp, Mosunetuzumab, Teclistamab, Cibisatamab, or Tarlatamab.

In some embodiments of the engineered NK cell of the disclosure, the BiTE is Blinatumomab.

In some embodiments of the engineered NK cell of the disclosure, the CD3e extracellular domain comprises a polypeptide encoded by a nucleic acid comprising SEQ ID NO. 23, or a functional fragment or variant thereof having at least 90% identity to SEQ ID 23.

In some embodiments of the engineered NK cell of the disclosure, the CD3g extracellular domain or the CD3d extracellular domain is linked to a second transmembrane domain, wherein the second transmembrane domain is linked to a second intracellular domain.

In some embodiments of the engineered NK cell of the disclosure, the first intracellular domain is linked to the extracellular domain of a second chimeric receptor by a second linker. In some embodiments, the second linker is a cleavable linker. In some embodiments, the second linker comprises SEQ ID NO. 54.

In some embodiments of the engineered NK cell of the disclosure, the first transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

In some embodiments of the engineered NK cell of the disclosure, the first transmembrane domain comprises a CD3e transmembrane domain.

In some embodiments of the engineered NK cell of the disclosure, the second transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

In some embodiments of the engineered NK cell of the disclosure, the second transmembrane domain comprises a CD3g transmembrane domain.

In some embodiments of the engineered NK cell of the disclosure, the first intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

In some embodiments of the engineered NK cell of the disclosure, the first intracellular signaling domain comprises a CD3z ITAM intracellular signaling domain.

In some embodiments of the engineered NK cell of the disclosure, the first intracellular signaling domain comprises a CD3z ITAM intracellular signaling domain comprising SEQ ID NO. 57.

In some embodiments of the engineered NK cell of the disclosure, the second intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

In some embodiments of the engineered NK cell of the disclosure, the second intracellular signaling domain comprises a 4-1BB intracellular signaling domain.

In some embodiments of the engineered NK cell of the disclosure, the second intracellular signaling domain comprises a 4-1BB intracellular signaling domain comprising SEQ ID NO. 59.

In some embodiments of the engineered NK cell of the disclosure, the 4-1BB intracellular signaling domain comprises a polypeptide encoded by a nucleic acid comprising SEQ ID NO. 7, or a functional fragment or variant thereof having at least 90% identity to SEQ ID NO. 7.

In some embodiments of the engineered NK cell of the disclosure, the CD79A intracellular signaling domain comprises a polypeptide encoded by a nucleic acid comprising SEQ ID NO: 1 (WT), a polypeptide encoded by a nucleic acid sequence comprising SEQ ID NO: 2 (CD79A (S197A, S203A, T209V), or a functional fragment or variant thereof having at least 90% identity to SEQ ID NO: 1 or SEQ ID NO: 2.

In some embodiments of the engineered NK cell of the disclosure, the CD79B intracellular signaling domain comprises a polypeptide encoded by a nucleic acid comprising SEQ ID NO: 3, or a functional fragment or variant thereof having at least 90% identity to SEQ ID NO: 3.

In some embodiments of the engineered NK cell of the disclosure, the 2B4 intracellular signaling domain comprises a polypeptide encoded by a nucleic acid comprising SEQ ID NO: 4, or a functional fragment or variant thereof having at least 90% identity to SEQ ID NO: 4.

In some embodiments of the engineered NK cell of the disclosure, the CD132 intracellular signaling domain comprises a polypeptide encoded by a nucleic acid comprising SEQ ID NO: 6, or a functional fragment or variant thereof having at least 90% identity to SEQ ID NO: 6.

In some embodiments of the engineered NK cell of the disclosure, the chimeric receptor comprises a CD3e extracellular domain linked to a CD3g extracellular domain, a CD16 transmembrane domain, and 2B4, CD79A, CD79B and CD132 intracellular domains.

In some embodiments of the engineered NK cell of the disclosure, the chimeric receptor comprises a CD3e extracellular domain linked to a CD3g extracellular domain, a CD3e transmembrane domain, and a CD3z intracellular domain.

In some embodiments of the engineered NK cell of the disclosure, the chimeric receptor comprises a CD3e extracellular domain, a CD3e transmembrane domain, and a CD3z intracellular domain.

In some embodiments of the engineered NK cell of the disclosure, the chimeric receptor comprises SEQ ID NO. 56.

In some embodiments of the engineered NK cell of the disclosure, the chimeric receptor comprises SEQ ID NO. 57.

In some embodiments of the engineered NK cell of the disclosure, the chimeric receptor comprises SEQ ID NO. 60.

In some embodiments of the engineered NK cell of the disclosure, the chimeric receptor comprises SEQ ID NO. 61.

In some embodiments of the engineered NK cell of the disclosure, the chimeric receptor comprises SEQ ID NO. 62.

In some embodiments of the engineered NK cell of the disclosure, the chimeric receptor comprises SEQ ID NO. 55.

In some embodiments of the engineered NK cell of the disclosure, the CD3g extracellular domain is linked to a CD3g transmembrane domain, and wherein the CD3g transmembrane domain is linked to a 4-1BB intracellular domain.

In some embodiments of the engineered NK cell of the disclosure, the engineered NK cell comprises a second chimeric receptor comprising a CD3g extracellular domain, a CD3g transmembrane domain, and a 4-1BB intracellular domain. In some embodiments, the second chimeric receptor comprises SEQ ID NO. 58. In some embodiments, the second chimeric receptor comprises SEQ ID NO. 63.

In some embodiments of the engineered NK cell of the disclosure, the chimeric receptor comprises a CD3e extracellular domain linked to a CD3d extracellular domain, a CD16 transmembrane domain, and 2B4, CD79A, CD79B and CD132 intracellular domains.

In some embodiments of the engineered NK cell of the disclosure, the chimeric receptor is expressed under the control of a promoter that is transcriptionally active in NK cells.

In some embodiments of the engineered NK cell of the disclosure, the promoter is MND.

In some embodiments of the engineered NK cell of the disclosure, the chimeric receptor comprises a signal peptide. In some embodiments, the signal peptide is the CD3e, CD3d, or CD3g signal peptide.

In some embodiments of the engineered NK cell of the disclosure, the chimeric receptor comprises a signal peptide. In some embodiments, the signal peptide is the CD3e, CD3d, or CD3g signal peptide.

In some embodiments of the engineered NK cell of the disclosure, the chimeric receptor comprises a P2A truncated CD34 protein on the terminal end of the chimeric receptor.

In some embodiments of the engineered NK cell of the disclosure, the cell is deficient for NKG2A and/or CD8 expression, activity, or signaling.

In some embodiments of the engineered NK cell of the disclosure, the NK cell is derived from cord blood, peripheral blood, an immortalized cell line, or an iPSC.

In some embodiments of the engineered NK cell of the disclosure, the NK cell is a memory-like NK cell.

The disclosure provides an engineered T cell comprising a chimeric receptor comprising a CD3 epsilon (CD3e) extracellular domain, a first transmembrane domain, and a first intracellular domain; where the first transmembrane domain does not comprise a CD3e transmembrane domain; where the engineered T cell does not comprise a T cell receptor (TCR).

The disclosure provides an engineered T cell comprising a chimeric receptor comprising a CD3 epsilon (CD3e) extracellular domain, a first transmembrane domain, and a first intracellular domain; where the first intracellular domain does not comprise a CD3e intracellular domain; where the engineered T cell does not comprise a T cell receptor (TCR).

In some embodiments of the engineered T cell of the disclosure, the T Cell Receptor Alpha chain (TRAC) gene is genetically modified or deleted.

In some embodiments of the engineered T cell of the disclosure, the CD3e extracellular domain is noncovalently associated with or linked to a CD3 gamma (CD3g) extracellular domain or a CD3 delta (CD3d) extracellular domain by a linker. In some embodiments, the linker comprises a polypeptide chain comprising a Gly Ser linker. In some embodiment, the linker comprises the polypeptide sequence set forth in SEQ ID NO. 52 or SEQ ID NO. 53.

In some embodiments of the engineered T cell of the disclosure, the CD3e extracellular domain is capable of binding to a T cell engager. In some embodiments, the T cell engager is selected from the list consisting of a bispecific T cell engager (BiTE), a trispecific T cell engager (TriTE), a TeTriTE, and a dual affinity retargeting antibody (DART). In some embodiments, the T cell engager is capable of binding to a target selected from PSMA, MAGE-A4, HER2, GPC3, GD2, FLT3, EPCAM, DLL3, CLDN 18.2, CD38, CD33, CD20, CD19, CD123, BCMA, B&-H7, and Mesothelin.

In some embodiments of the engineered T cell of the disclosure, the CD3e extracellular domain is capable of binding to a bispecific T cell engager (BiTE). In some embodiments, the BiTE is Blinatumomab, Tebentafusp, Mosunetuzumab, Teclistamab, Cibisatamab, or Tarlatamab. In some embodiments, the BiTE is Blinatumomab.

In some embodiments of the engineered T cell of the disclosure, the CD3e extracellular domain comprises a polypeptide encoded by a nucleic acid comprising SEQ ID NO. 23, or a functional fragment or variant thereof having at least 90% identity to SEQ ID 23.

In some embodiments of the engineered T cell of the disclosure, the CD3g extracellular domain or the CD3d extracellular domain is linked to a second transmembrane domain, and the second transmembrane domain is linked to a second intracellular domain.

In some embodiments of the engineered T cell of the disclosure, the first transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

In some embodiments, the first transmembrane domain comprises a CD3e transmembrane domain.

In some embodiments of the engineered T cell of the disclosure, the second transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains. In some embodiments, the second transmembrane domain comprises a CD3g transmembrane domain.

In some embodiments of the engineered T cell of the disclosure, the first intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains. In some embodiments, the first intracellular signaling domain comprises a CD3z ITAM intracellular signaling domain. In some embodiments of the engineered T cell of the disclosure, the second intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains. In some embodiments, the second intracellular signaling domain comprises a 4-1BB intracellular signaling domain. In some embodiments, the 4-1BB intracellular signaling domain comprises a polypeptide encoded by a nucleic acid comprising SEQ ID NO. 7, or a functional fragment or variant thereof having at least 90% identity to SEQ ID NO. 7. In some embodiments, the CD79A intracellular signaling domain comprises a polypeptide encoded by a nucleic acid comprising SEQ ID NO: 1 (WT), a polypeptide encoded by a nucleic acid sequence comprising SEQ ID NO: 2 (CD79A (S197A, S203A, T209V), or a functional fragment or variant thereof having at least 90% identity to SEQ ID NO: 1 or SEQ ID NO: 2. In some embodiments, the CD79B intracellular signaling domain comprises a polypeptide encoded by a nucleic acid comprising SEQ ID NO: 3, or a functional fragment or variant thereof having at least 90% identity to SEQ ID NO: 3. In some embodiments, the 2B4 intracellular signaling domain comprises a polypeptide encoded by a nucleic acid comprising SEQ ID NO: 4, or a functional fragment or variant thereof having at least 90% identity to SEQ ID NO: 4. In some embodiments, the CD132 intracellular signaling domain comprises a polypeptide encoded by a nucleic acid comprising SEQ ID NO: 6, or a functional fragment or variant thereof having at least 90% identity to SEQ ID NO: 6.

In some embodiments of the engineered T cell of the disclosure, the chimeric receptor comprises a CD3e extracellular domain linked to a CD3g extracellular domain, a CD16 transmembrane domain, and 2B4, CD79A, CD79B and CD132 intracellular domains. In some embodiments of the engineered T cell of the disclosure, the chimeric receptor comprises a CD3e extracellular domain linked to a CD3g extracellular domain, a CD3e transmembrane domain, and a CD3z intracellular domain. In some embodiments, the CD3g extracellular domain is linked to a CD3g transmembrane domain, and the CD3g transmembrane domain is linked to a 4-1BB intracellular domain.

In some embodiments of the engineered T cell of the disclosure, the chimeric receptor comprises a CD3e extracellular domain linked to a CD3d extracellular domain, a CD16 transmembrane domain, and 2B4, CD79A, CD79B and CD132 intracellular domains.

In some embodiments of the engineered T cell of the disclosure, the chimeric receptor is expressed under the control of a promoter that is transcriptionally active in T cells. In some embodiments, the promoter is MND.

In some embodiments of the engineered T cell of the disclosure, the chimeric receptor comprises a P2A truncated CD34 protein on the terminal end of the chimeric receptor.

In some embodiments of the engineered T cell of the disclosure, the cell is deficient for NKG2A and/or CD8 expression, activity, or signaling.

In some embodiments of the engineered T cell of the disclosure, the T cell is derived from cord blood, peripheral blood, an immortalized cell line, or an iPSC.

In some embodiments of the engineered T cell of the disclosure, the T cell is a memory T cell.

In some embodiments, the disclosure provides a chimeric receptor capable of being expressed in a natural killer (NK) cell, wherein the receptor comprises: (a) a CD3 epsilon (CD3e) extracellular domain; (b) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and (c) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, and IRE1a.

In some embodiments, the disclosure provides a chimeric receptor capable of being expressed in a natural killer (NK) cell, wherein the receptor comprises: (a) a CD3 epsilon (CD3e) extracellular domain; (b) a first transmembrane domain selected from the group consisting of: CD3d, CD3e, CD3g, CD16, NKG2D, DAP10, FcγRIIIa, NKp44, NKp30, NKp46, actKIR, NKG2C, CD8α, and IL15; and (c) a first intracellular signaling domains selected from the group consisting of: CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, and IRE1a.

In some embodiments, the disclosure provides a chimeric receptor capable of being expressed in a natural killer (NK) cell, where the receptor includes (a) a CD3 epsilon (CD3e) extracellular domain, (b) a first transmembrane domain selected from the group consisting of: CD3d, CD3e, CD3g, CD16, NKG2D, DAP10, FcγRIIIa, NKp44, NKp30, NKp46, actKIR, NKG2C, CD8α, and IL15, and (c) a first intracellular signaling domain selected from the group consisting of: CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, and IRE1a.

In some embodiments, the disclosure provides a chimeric receptor capable of being expressed in a natural killer (NK) cell, where the receptor includes a CD3 epsilon (CD3e) extracellular domain, a first transmembrane domain selected from the group consisting of: CD3d, CD3g, CD16, NKG2D, DAP10, FcγRIIIa, NKp44, NKp30, NKp46, actKIR, NKG2C, CD8α, and IL15, and a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

In some embodiments of the chimeric receptor of the disclosure, the chimeric receptor comprises SEQ ID NO. 56.

In some embodiments of the chimeric receptor of the disclosure, the chimeric receptor comprises SEQ ID NO. 57.

In some embodiments of the chimeric receptor of the disclosure, the chimeric receptor comprises SEQ ID NO. 60.

In some embodiments of the chimeric receptor of the disclosure, the chimeric receptor comprises SEQ ID NO. 61.

In some embodiments of the chimeric receptor of the disclosure, the chimeric receptor comprises SEQ ID NO. 62.

In some embodiments of the chimeric receptor of the disclosure, the chimeric receptor comprises SEQ ID NO. 55.

In some embodiments of the chimeric receptor of the disclosure, the CD3e extracellular domain is noncovalently associated with or linked to a CD3 gamma (CD3g) extracellular domain or a CD3 delta (CD3d) extracellular domain by a linker.

In some embodiments of the chimeric receptor of the disclosure, the linker comprises a polypeptide chain comprising a Gly Ser linker.

In some embodiments of the chimeric receptor of the disclosure, the linker comprises the polypeptide sequence set forth in SEQ ID NO. 52 or SEQ ID NO. 53.

In some embodiments of the chimeric receptor of the disclosure, the CD3e extracellular domain is capable of binding to a bispecific T cell engager (BiTE).

In some embodiments of the chimeric receptor of the disclosure, the BiTE is selected from Blinatumomab, Tebentafusp, Mosunetuzumab, Teclistamab, Cibisatamab, and Tarlatamab.

In some embodiments of the chimeric receptor of the disclosure, the BiTE is Blinatumomab.

In some embodiments of the chimeric receptor of the disclosure, the CD3e extracellular domain comprises a polypeptide encoded by a nucleic acid comprising SEQ ID NO. 23, or a functional fragment or variant thereof having at least 90% identity to SEQ ID NO. 23.

In some embodiments of the chimeric receptor of the disclosure, the first transmembrane domain comprises a CD3e transmembrane domain.

In some embodiments of the chimeric receptor of the disclosure, the CD3g extracellular domain or the CD3d extracellular domain is linked to a second transmembrane domain, wherein the second transmembrane domain is linked to a second intracellular domain.

In some embodiments of the chimeric receptor of the disclosure, the first intracellular domain is linked to the extracellular domain of a second chimeric receptor by a second linker. In some embodiments, the second linker is a cleavable linker. In some embodiments, the second linker comprises SEQ ID NO. 54.

In some embodiments of the chimeric receptor of the disclosure, the second transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

In some embodiments of the chimeric receptor of the disclosure, the second transmembrane domain comprises a CD3g transmembrane domain.

In some embodiments of the chimeric receptor of the disclosure, the first intracellular signaling domain comprises a CD3z intracellular signaling domain.

In some embodiments of the chimeric receptor of the disclosure, the first intracellular signaling domain comprises a CD3z intracellular signaling domain comprising SEQ ID NO. 57.

In some embodiments of the chimeric receptor of the disclosure, the second intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

In some embodiments of the chimeric receptor of the disclosure, the second intracellular signaling domain comprises a 4-1BB intracellular signaling domain.

In some embodiments of the chimeric receptor of the disclosure, the second intracellular signaling domain comprises a 4-1BB intracellular signaling domain comprising SEQ ID NO. 59.

In some embodiments of the chimeric receptor of the disclosure, the 4-1BB intracellular signaling domain comprises a polypeptide encoded by a nucleic acid comprising SEQ ID NO. 7, or a functional fragment or variant thereof having at least 90% identity to SEQ ID NO. 7.

In some embodiments of the chimeric receptor of the disclosure, the CD79A intracellular signaling domain comprises a polypeptide encoded by a nucleic acid comprising SEQ ID NO: 1 (WT), a polypeptide encoded by a nucleic acid sequence comprising SEQ ID NO: 2 (CD79A (S197A, S203A, T209V), or a functional fragment or variant thereof having at least 90% identity to SEQ ID NO: 1 or SEQ ID NO: 2.

In some embodiments of the chimeric receptor of the disclosure, the CD79B intracellular signaling domain comprises a polypeptide encoded by a nucleic acid comprising SEQ ID NO: 3, or a functional fragment or variant thereof having at least 90% identity to SEQ ID NO: 3.

In some embodiments of the chimeric receptor of the disclosure, the 2B4 intracellular signaling domain comprises a polypeptide encoded by a nucleic acid comprising SEQ ID NO: 4, or a functional fragment or variant thereof having at least 90% identity to SEQ ID NO: 4.

In some embodiments of the chimeric receptor of the disclosure, the CD132 intracellular signaling domain comprises a polypeptide encoded by a nucleic acid comprising SEQ ID NO: 6, or a functional fragment or variant thereof having at least 90% identity to SEQ ID NO: 6.

In some embodiments of the chimeric receptor of the disclosure, the chimeric receptor comprises a CD3e extracellular domain linked to a CD3g extracellular domain, a CD16 transmembrane domain, and 2B4, CD79A, CD79B and CD132 intracellular domains.

In some embodiments of the chimeric receptor of the disclosure, the chimeric receptor comprises a CD3e extracellular domain linked to a CD3g extracellular domain, a CD3e transmembrane domain, and a CD3z intracellular domain.

In some embodiments of the chimeric receptor of the disclosure, the chimeric receptor comprises a CD3e extracellular domain, a CD3e transmembrane domain, and a CD3z intracellular domain.

In some embodiments of the chimeric receptor of the disclosure, the CD3g extracellular domain is linked to a CD3g transmembrane domain, and wherein the CD3g transmembrane domain is linked to a 4-1BB intracellular domain.

In some embodiments of the chimeric receptor of the disclosure, the chimeric receptor comprises a CD3e extracellular domain linked to a CD3d extracellular domain, a CD16 transmembrane domain, and 2B4, CD79A, CD79B and CD132 intracellular domains.

In some embodiments of the chimeric receptor of the disclosure, the chimeric receptor is

In some embodiments of the chimeric receptor of the disclosure, the chimeric receptor is expressed under the control of a promoter that is transcriptionally active in NK cells.

In some embodiments of the chimeric receptor of the disclosure, the promoter is MND.

In some embodiments of the chimeric receptor of the disclosure, the chimeric receptor comprises a signal peptide. In some embodiments, the signal peptide is the CD3e, CD3d, or CD3g signal peptide.

In some embodiments of the chimeric receptor of the disclosure, the chimeric receptor comprises a P2A truncated CD34 protein on the terminal end of the chimeric receptor.

In some embodiments of the chimeric receptor of the disclosure, the chimeric receptor is noncovalently associated or linked to a second chimeric receptor comprising a CD3g extracellular domain, a CD3g transmembrane domain, and a 4-1BB intracellular domain. In some embodiments, the second chimeric receptor comprises SEQ ID NO. 58. In some embodiments, the second chimeric receptor comprises SEQ ID NO. 63.

In some embodiments, the disclosure provides a vector comprising a nucleic acid encoding a chimeric receptor of the disclosure.

In some embodiments of the vector of the disclosure, the vector is a viral vector.

In some embodiments of the vector of the disclosure, the vector is a retroviral or lentiviral vector.

In some embodiments, the disclosure provides a bicistronic chimeric receptor comprising a first and a second chimeric receptor, wherein the first and the second chimeric receptors are co-expressed by a bicistronic vector, wherein the first chimeric receptor is a chimeric receptor of the disclosure.

In some embodiments, the disclosure provides a bicistronic chimeric receptor comprising a first and a second chimeric receptor, where the first and the second chimeric receptors are co-expressed by a bicistronic vector, the first chimeric receptor comprises a CD3e extracellular domain, and the second chimeric receptor comprises a CD3g or a CD3d extracellular domain.

In some embodiments of the bicistronic chimeric receptor of the disclosure, the first chimeric receptor comprises: (a) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and (b) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

In some embodiments of the bicistronic chimeric receptor of the disclosure, the first chimeric receptor comprises: (a) a first transmembrane domain selected from the group consisting of: CD3e, CD3g, CD16, NKG2D, DAP10, FcγRIIIa, NKp44, NKp30, NKp46, actKIR, NKG2C, CD8α, and IL15; and (b) a first intracellular signaling domain selected from the group consisting of: CD3d, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

In some embodiments of the bicistronic chimeric receptor of the disclosure, the first chimeric receptor comprises a CD3e extracellular domain, a CD3e transmembrane domain, and a CD3zeta intracellular domain; and the second chimeric receptor comprises a CD3g extracellular domain, a CD3g transmembrane domain, and a 41BB intracellular domain.

In some embodiments of the bicistronic chimeric receptor of the disclosure, the first chimeric receptor comprises a CD3e extracellular domain, a CD3e transmembrane domain, and CD3zeta and 2B4 intracellular domains; and the second chimeric receptor comprises a CD3g extracellular domain, a CD3g transmembrane domain, and a OX40 intracellular domain.

In some embodiments of the bicistronic chimeric receptor of the disclosure, the first chimeric receptor is linked to the second chimeric receptor by a second linker. In some embodiments, the intracellular domain of the first chimeric receptor is linked to the extracellular domain of the second chimeric receptor by the second linker. In some embodiments, the second linker is a cleavable linker. In some embodiments, the second linker comprises SEQ ID NO. 54.

In some embodiments of the bicistronic chimeric receptor of the disclosure, the first chimeric receptor comprises a CD3e extracellular domain, a CD3e transmembrane domain, and a CD3e intracellular domain; wherein the second chimeric receptor comprises a CD3g extracellular domain, a CD3g transmembrane domain, and a CD3g intracellular domain.

In some embodiments of the bicistronic chimeric receptor of the disclosure, the first chimeric receptor comprises a CD3e extracellular domain, a CD3e transmembrane domain, and CD79A and 2B4 intracellular domains; wherein the second chimeric receptor comprises a CD3g extracellular domain, a CD3g transmembrane domain, and a CD79B intracellular domain.

In some embodiments of the bicistronic chimeric receptor of the disclosure, the first chimeric receptor comprises a CD3e extracellular domain, a CD3e transmembrane domain, and CD79A, CD132 and 2B4 intracellular domains; wherein the second chimeric receptor comprises a CD3g extracellular domain, a CD3g transmembrane domain, and CD79B and IL2Rbeta intracellular domains.

In some embodiments of the bicistronic chimeric receptor of the disclosure, the first chimeric receptor comprises a CD3e extracellular domain, a CD3e transmembrane domain, and CD79A, CD132 and 2B4 intracellular domains; wherein the second chimeric receptor comprises a CD3d extracellular domain, a CD3d transmembrane domain, and CD79B and IL2Rbeta intracellular domains.

In some embodiments of the bicistronic chimeric receptor of the disclosure, the first chimeric receptor comprises a CD3e extracellular domain, a CD3e transmembrane domain, and a CD3zeta intracellular domain; wherein the second chimeric receptor comprises a CD3g extracellular domain, a CD3g transmembrane domain, and a 41BB intracellular domain.

In some embodiments of the bicistronic chimeric receptor of the disclosure, the first intracellular signaling domain comprises a CD3z ITAM intracellular signaling domain comprising SEQ ID NO. 57.

In some embodiments of the bicistronic chimeric receptor of the disclosure, the second intracellular signaling domain comprises a 4-1BB intracellular signaling domain comprising SEQ ID NO. 59.

In some embodiments of the bicistronic chimeric receptor of the disclosure, the bicistronic chimeric receptor comprises SEQ ID NO. 56.

In some embodiments of the bicistronic chimeric receptor of the disclosure, the bicistronic chimeric receptor comprises SEQ ID NO. 57.

In some embodiments of the bicistronic chimeric receptor of the disclosure, the bicistronic chimeric receptor comprises SEQ ID NO. 60.

In some embodiments of the bicistronic chimeric receptor of the disclosure, the bicistronic chimeric receptor comprises SEQ ID NO. 61.

In some embodiments of the bicistronic chimeric receptor of the disclosure, the bicistronic chimeric receptor comprises SEQ ID NO. 62.

In some embodiments of the bicistronic chimeric receptor of the disclosure, the bicistronic chimeric receptor comprises SEQ ID NO. 55.

In some embodiments of the bicistronic chimeric receptor of the disclosure, the first chimeric receptor comprises a CD3e extracellular domain, a CD3e transmembrane domain, and CD3zeta and CD132 intracellular domains; wherein the second chimeric receptor comprises a CD3g extracellular domain, a CD3g transmembrane domain, and 41BB and IL2Rb intracellular domains.

In some embodiments of the bicistronic chimeric receptor of the disclosure, the bicistronic chimeric receptor is expressed under the control of a promoter that is transcriptionally active in NK cells. In some embodiments, the promoter is MND.

In some embodiments of the bicistronic chimeric receptor of the disclosure, the chimeric receptor comprises a signal peptide. In some embodiments, the signal peptide is the CD3e, CD3d, or CD3g signal peptide.

In some embodiments of the bicistronic chimeric receptor of the disclosure, the bicistronic chimeric receptor comprises a P2A truncated CD34 protein on the terminal end of the chimeric receptor.

In some embodiments, the disclosure provides a bicistronic vector comprising a nucleic acid encoding a bicistronic chimeric receptor of the disclosure.

In some embodiments of the bicistronic vector of the disclosure, the bicistronic vector is a viral vector.

In some embodiments of the bicistronic vector of the disclosure, the bicistronic vector is a retroviral or lentiviral vector.

In some embodiments, the disclosure provides a tricistronic chimeric receptor comprising a first, a second, and a third chimeric receptor, where the first, the second, and the third chimeric receptors are co-expressed by a tricistronic vector, where the first chimeric receptor comprises a CD3e extracellular domain, the second chimeric receptor comprises a CD3g extracellular domain, and the third chimeric receptor comprises a CD3d extracellular domain.

In some embodiments of the tricistronic chimeric receptor of the disclosure, the first chimeric receptor comprises: (a) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and (b) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

In some embodiments of the tricistronic chimeric receptor of the disclosure, the first chimeric receptor comprises: (a) a first transmembrane domain selected from the group consisting of: CD3e, CD3g, CD16, NKG2D, DAP10, FcγRIIIa, NKp44, NKp30, NKp46, actKIR, NKG2C, CD8α, and IL15; and (b) a first intracellular signaling domain selected from the group consisting of: CD3d, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

In some embodiments of the tricistronic chimeric receptor of the disclosure, the first chimeric receptor comprises a CD3e extracellular domain, a CD3e transmembrane domain, and a CD3z intracellular domain; the second chimeric receptor comprises a CD3d extracellular domain, a CD3d transmembrane domain, and a 2B4 intracellular domain; and the third chimeric receptor comprises a CD3g extracellular domain, a CD3g transmembrane domain, and a 4-1BB intracellular domain.

In some embodiments, the disclosure provides a tricistronic vector comprising a nucleic acid encoding a tricistronic chimeric receptor of the disclosure.

In some embodiments of the tricistronic vector of the disclosure, the tricistronic vector is a viral vector.

In some embodiments of the tricistronic vector of the disclosure, the tricistronic vector is a retroviral or lentiviral vector.

In some embodiments, the disclosure provides an engineered NK cell comprising a chimeric receptor of the disclosure.

In some embodiments, the disclosure provides an engineered NK cell comprising a bicistronic chimeric receptor of the disclosure.

In some embodiments, the disclosure provides an engineered NK cell comprising a tricistronic chimeric receptor of the disclosure.

In some embodiments, the disclosure provides an engineered NK cell comprising a vector of the disclosure.

In some embodiments, the disclosure provides an engineered NK cell comprising a bicistronic vector of the disclosure.

In some embodiments, the disclosure provides an engineered NK cell comprising a tricistronic vector of the disclosure.

In some embodiments, the disclosure provides an engineered NK cell comprising a chimeric receptor of the disclosure and a vector of the disclosure.

In some embodiments, the disclosure provides an engineered NK cell comprising a bicistronic chimeric receptor of the disclosure and a bicistronic vector of the disclosure.

In some embodiments, the disclosure provides an engineered NK cell comprising a tricistronic chimeric receptor of the disclosure and a tricistronic vector of the disclosure.

In some embodiments of the engineered NK cell of the disclosure, the cell is deficient for NKG2A and/or CD8 expression, activity, or signaling.

In some embodiments of the engineered NK cell of the disclosure, the NK cell is derived from cord blood, peripheral blood, an immortalized cell line, or an iPSC.

In some embodiments of the engineered NK cell of the disclosure, the NK cell is a memory-like NK cell.

In some embodiments of the engineered NK cell of the disclosure, the extracellular domain of the chimeric receptor, bicistronic chimeric receptor, or tricistronic chimeric receptor is capable of binding to a T cell engager. In some embodiments, the T cell engager is selected from the list consisting of a bispecific T cell engager (BiTE), a trispecific T cell engager (TriTE), a TeTriTE, and a dual affinity retargeting antibody (DART).

In some embodiments, the disclosure provides an engineered T cell comprising a chimeric receptor of the disclosure.

In some embodiments, the disclosure provides an engineered T cell comprising a bicistronic chimeric receptor of the disclosure.

In some embodiments, the disclosure provides an engineered T cell comprising a tricistronic chimeric receptor of the disclosure.

In some embodiments, the disclosure provides an engineered T cell comprising a vector of the disclosure.

In some embodiments, the disclosure provides an engineered T cell comprising a bicistronic vector of the disclosure.

In some embodiments, the disclosure provides an engineered T cell comprising a tricistronic vector of the disclosure.

In some embodiments, the disclosure provides an engineered T cell comprising a chimeric receptor of the disclosure and a vector of the disclosure.

In some embodiments, the disclosure provides an engineered T cell comprising a bicistronic chimeric receptor of the disclosure and a bicistronic vector of the disclosure.

In some embodiments, the disclosure provides an engineered T cell comprising a tricistronic chimeric receptor of the disclosure and a tricistronic vector of the disclosure.

In some embodiments of the engineered T cell of the disclosure, the T Cell Receptor Alpha chain (TRAC) gene is genetically modified or deleted.

In some embodiments of the engineered T cell of the disclosure, the cell is deficient for NKG2A and/or CD8 expression, activity, or signaling.

In some embodiments of the engineered T cell of the disclosure, the T cell is derived from cord blood, peripheral blood, an immortalized cell line, or an iPSC.

In some embodiments of the engineered T cell of the disclosure, the T cell is a memory T cell.

In some embodiments of the engineered T cell of the disclosure, the extracellular domain of the chimeric receptor, bicistronic chimeric receptor, or tricistronic chimeric receptor is capable of binding to a T cell engager. In some embodiments, the T cell engager is selected from the list consisting of a bispecific T cell engager (BiTE), a trispecific T cell engager (TriTE), a TeTriTE, and a dual affinity retargeting antibody (DART).

In some embodiments, the disclosure provides a method of inducing an immune response to a disease in a subject in need thereof comprising administering to the subject an engineered NK cell of the disclosure.

In some embodiments of the methods of the disclosure, the method comprises administering to the subject a BiTE.

In some embodiments of the methods of the disclosure, the BiTE is selected from Blinatumomab, Tebentafusp, Mosunetuzumab, Teclistamab, Cibisatamab, and Tarlatamab.

In some embodiments of the methods of the disclosure, the BiTE is Blinatumomab.

In some embodiments of the methods of the disclosure, the disease is cancer, an autoimmune condition, or an infectious disease.

In some embodiments of the methods of the disclosure, the disease is cancer.

In some embodiments of the methods of the disclosure, the cancer comprises a solid tumor.

In some embodiments of the methods of the disclosure, the cancer is a hematologic cancer.

### BRIEF DESCRIPTION OF THE DRAWINGS

FIG. 1 provides a schematic diagram showing a chimeric receptor-expressing NK cell using bispecific T cell engagers (BiTEs) to target cancer cells.

FIG. 2 provides a graph showing the number of available, clinical bispecific T cell engagers (TCEs) compared to the number of NK cell engagers (NKEs).

FIG. 3A provides a chart showing the number of exemplary clinical NKEs that can be used in combination with CD3-NK cells, their tumor target antigens, and the category of the tumor targets.

FIG. 3B provides a chart showing the number of exemplary clinical TCEs that can be used in combination with CD3-NK cells, their tumor target antigens, and the category of the tumor targets.

FIG. 4 provides a set of schematic diagrams showing exemplary embodiments of killing target cells using TCEs with endogenous T cells, using TCEs with endogenous T cells and CD3-NK cells, and using TCEs in combination with CD3-NK cells after conditioning to deplete endogenous T cells.

FIG. 5 provides a schematic diagram for stabilizing cell surface expression of CD3e as a single chain variable fragment (Fv) by co-expression with other CD3 members (1) through a linker (2) through altering the highly conserved CXXC motif found on the membrane proximal end of CD3e, CD3g or CD3d (3) and through varying the position of CD3e as the membrane proximal or distal domain within the extracellular region (4).

FIG. 6A and FIG. 6B provide schematic diagrams showing chimeric receptor constructs and bicistronic chimeric receptor constructs for expression of CD3e on the cell surface.

FIG. 7A provides a diagram showing two chimeric receptor constructs WU76E and WU71A. WU76E is a bicistronic receptor expressing a first chimeric receptor with a CD3e extracellular domain, a CD3e transmembrane domain, and a CD3zeta intracellular domain; and a second chimeric receptor with a CD3g extracellular domain, a CD3g transmembrane domain, and a 41BB intracellular domain. WU71A is a chimeric receptor with an anti-CD19 scFv extracellular domain, a CD16 transmembrane domain, and 2B4 and CD79A/B intracellular domains.

FIG. 7B provides graphs showing the fluorescent intensity of cell surface CD3 expression by 293T-X cells transduced with lentivirus plasmid constructs encoding WU76E and WU71A. Expression was assessed by flow cytometry staining using 5 different anti-CD3 antibody clones: OKT3, UCHT1, TR66, HIT3a, or SK7.

FIG. 8 provides representative plots of flow cytometry staining of cell surface CD3 and CD56 on NK cells transduced with WU76E. Cells were analyzed on day 6 and day 14 of manufacturing.

FIG. 9 provides a graph showing the fold expansion of non-transduced NK cells (NK101), NK cells transduced with WU71A and NK cells transduced with WU76E over the course of 14 days in culture.

FIG. 10A provides representative plots of flow cytometry staining of cell surface CD56 and CD3 on three populations of cells: WU76E-NK cells prior to CD3 positive selection using STEMCELL human CD3 Positive Selective kits (Presort), WU76E-NK cells that were positively selected by the kit (Post Sort) and the flow through population that was not selected.

FIG. 10B provides representative plots of flow cytometry staining of cell surface CD56 and CD3 on WU76E-NK cells that were positively selected using STEMCELL human CD3 Positive Selection kits after 24 hours of culturing and on T cells.

FIG. 11 provides the A Sartorius Incucyte scan parameters used to quantify GFP+NALM6 cancer cells and representative images of the phase and green signals captured by A Sartorius Incucyte, as well as illustrative images of the green object mask analysis performed by the A Sartorius Incucyte software to identify viable NALM6 cells.

FIG. 12 provides a graph showing the cell growth of NALM6 cells cultured in the presence of different concentrations of Blinatumomab. Cell growth was measured using A Sartorius Incucyte and normalized to the starting number of cells.

FIG. 13 provides graphs showing the quantification of viable NALM6 cells remaining in each test condition over the course of 120 hours. NALM6 cells were co-cultured with different ratios of naïve T cells in the presence of 5 g/mL, 312 ng/mL, 78 ng/mL or Ong/mL of Blinatumomab and growth of NALM6 cells were measured via A Sartorius Incucyte and normalized to the number of starting cells.

FIG. 14 provides a graph showing the quantification of viable NALM6 cells remaining in each test condition over the course of 72 hours as measured by A Sartorius Incucyte. Isolated NK cells were primed, expanded and transduced with lentivirus expressing the WU76E or WU71A construct (WU76E-NK cells and WU71A-NK cells, respectively) and subsequently positively selected for CD3 on day 14 of culturing. The NALM6 cells, WU76E-NK cells, and WU71A-NK cells were used on day 15 for cytotoxicity assays and tested under six different conditions:
(1) target only—NALM6 cells alone,
(2) target+Blina, NALM6 cells with 100 ng/mL of Blinatumomab,
(3) T Cell—NALM6 cells co-cultured with naïve, isolated CD3+ T cells at a 1:3 effector to target ratio in the presence of 100 ng/mL of Blinatumomab
(4) NK101—NALM6 cells co-cultured with NK101 at a 1:3 effector to target ratio in the presence of 100 ng/mL of Blinatumomab
(5) CAR19-NK—NALM6 cells co-cultured with WU71A-NK cells at a 1:3 effector to target ratio in the presence of 100 ng/mL of Blinatumomab
(6) CD3CAR-NK—NALM6 cells co-cultured with WU76E-NK cells at a 1:3 effector to target ratio in the presence of 100 ng/mL of Blinatumomab
   All T and NK cells are isolated from the same donor and the assay was carried out in the presence of 100 IU/mL of IL-2 in complete RPMI.

FIG. 15 provides a graph showing the quantification of viable NALM6 cells remaining over the course of 72 hours as measured by A Sartorius Incucyte in each of the following test conditions:
(1) Target only—NALM6 cells alone
(2) Target+BiTE—NALM6 cells with 200 ng/mL of Blinatumomab
(3) NTD—NALM6 cells co-cultured with untransduced NK cells (NTD)
(4) NTD+BiTE—NALM6 cells co-cultured with untransduced NK cells at a 1:1 effector to target ratio in the presence of 200 ng/mL of Blinatumomab
(5) WU76E—NALM6 cells co-cultured with WU76E-NK cells at a 1:1 effector to target ratio
(6) WU76E+BiTE—NALM6 cells co-cultured with WU76E-NK cells at a 1:1 effector to target ratio in the presence of 200 ng/mL of Blinatumomab

FIG. 16 provides a schematic showing the intracellular, transmembrane, and extracellular domains of WU76Z, a tricistronic construct encoding three chains.

FIG. 17 provides a graph showing the quantification of viable NALM6 cells remaining over the course of 72 hours as measured by A Sartorius Incucyte in each of the following test conditions:
(1) Target—NALM6 cells alone
(2) Target+BiTE—NALM6 cells with 200 ng/mL of Blinatumomab
(3) NTD—NALM6 cells co-cultured with untransduced NK cells
(4) NTD+BiTE—NALM6 cells co-cultured with untransduced NK cells at a 1:1 effector to target ratio in the presence of 200 ng/mL of Blinatumomab
(5) WU76Z—NALM6 cells co-cultured with WU76Z-NK cells at a 1:1 effector to target ratio
(6) WU76Z+BiTE—NALM6 cells co-cultured with WU76Z-NK cells at a 1:1 effector to target ratio in the presence of 200 ng/mL of Blinatumomab

FIG. 18 provides a schematic showing the intracellular, transmembrane, and extracellular domains of WU76UB, a bicistronic construct.

FIG. 19 provides graphs showing the quantification by flow cytometry of the amount of different T cell engagers that bind to WU76UB-NK cells. The T cell engagers tested were biosimilars for blinatumomab and tarlatamab, EGFR×CD3 and CEA×CD3 T cell engagers, at two different concentrations: 20 ng/ml and 200 ng/ml.

FIG. 20A and FIG. 20B show the cytotoxicity of WU76UB-NK cells against target NALM6 cells and HT144 cells. FIG. 20A provides a graph showing the quantification of viable NALM6 target cells remaining, as a percentage of starting targets, over the course of 72 hours as measured by A Sartorius Incucyte in each of the following test conditions:
(1) Target—NALM6 cells alone
(2) Target+BiTE—NALM6 cells with 200 ng/mL of Blinatumomab
(3) WU76UB—NALM6 cells co-cultured with WU76UB-NK cells at a 1:1 effector to target ratio
(4) WU76UB+BiTE—NALM6 cells co-cultured with WU76UB-NK cells at a 1:1 effector to target ratio in the presence of 200 ng/mL of Blinatumomab

FIG. 20B provides a graph showing the quantification of viable HT144 target cells remaining in each of the following test conditions:
(1) Target—HT144 cells alone
(2) Target+BiTE—HT144 cells with 200 ng/mL of EGFR BiTE
(3) WU76UB—HT144 cells co-cultured with WU76UB-NK cells at a 1:1 effector to target ratio
(4) WU76UB+BiTE—HT144 cells co-cultured with WU76UB-NK cells at a 1:1 effector to target ratio in the presence of 200 ng/mL of EGFR BiTE

FIG. 21A and FIG. 21B show the quantification of phosphor-S6 (pS6) levels in WU76UB-NK cells, WU76Z-NK cells, or untransduced NK cells (NTD). FIG. 21A provides quantification of phosphor-S6 levels by flow cytometry in untransduced NK cells (NTD) and WU76UB-NK cells when stimulated with phorbol myristate acetate and ionomycin (PMA:J) or NALM6 cells and Blinatumomab (NALM6+BiTE) or left unstimulated (Unstim). FIG. 21B shows the fold change of pS6 levels in untransduced NK cells, WU76UB-NK cells, and WU76Z-NK cells after coculturing with NALM6 cells in the absence (Nalm6) or presence (Nalm6+BiTE) of Blinatumomab.

FIG. 22A and FIG. 22B show the quantification of NFAT signaling in Jurkat NFAT-GFP reporter cells. FIG. 22A shows the quantification of cell surface CD3 expression by (1) wildtype Jurkat cells (Jurkat WT), (2) T cell receptor alpha chain (TRAC) knockout Jurkat cells (Jurkat TRAC KO), and Jurkat TRAC KO cells transduced with each of the following constructs: (3) WU76B (76B), (4) WU76E (76E), (5) WU76UB (76UB), and (6) WU76Z (76Z).

FIG. 22B shows the quantification of NFAT signaling in each of the 6 population of cells shown in FIG. 22A when cocultured with SKOV3 tumor cells in the absence (SKOV3) or presence of a EGFR targeting T cell engager (SKOV3+EGFR BiTE).

FIG. 23A and FIG. 23B show the quantification of IFNγ production by WU76UB-NK cells, WU76Z-NK cells, or untransduced NK cells (NTD). FIG. 23A shows the flow cytometry quantification of intracellular staining of IFNγ in untransduced NK cells (NTD) and WU76UB-NK cells in the following conditions: (1) stimulated with PMA and ionomycin, (2) cocultured with NALM6 cells in the presence of Blinatumomab (E+T+BiTE), (3) cocultured with NALM6 cells (E+T), and (4) unstimulated (E Only). FIG. 23B provides a heatmap showing the median fluorescent intensity (MFI) of IFNγ expression in untransduced NK cells (NTD), WU76UB-NK cells, and WU76Z-NK cells, quantified by flow cytometry.

FIG. 24 shows the quantification of granzyme B by ELISA in the supernatant of untransduced NK cell (NTD) and WU76UB-NK cell cultures, each cocultured with NALM6 cells in the absence (Target Only) or presence (Target+BiTE) of Blinatumomab.

FIG. 25 provides representative plots of flow cytometry staining of cell surface TCRαβ and CD3 expression on unmodified T cells (T Cell), T cells with TRAC deletion (TRAC^{KO} T Cell), and T cells with TRAC deletion that were transduced with the WU76UB chimeric receptor (TRACKO T Cell+WU76UB).

### DETAILED DESCRIPTION

The present disclosure relates to NK cells modified to express a chimeric receptor for use in combination with bispecific targeting agents that bind CD3 and methods of making and using the same. Natural killer (NK) cells are an immune cell type being used to develop cellular therapeutics for treatment of cancer and other diseases affecting the immune system. Inherently, NK cells have properties that make them attractive for therapeutic use, including low toxicity profile and broad tumor killing ability. However, some target cells are resistant to endogenous NK cell killing. To circumvent this problem, NK cells can be administered in conjunction with targeting molecules that link the NK cell and the tumor cells. Bispecific engagers are engineered proteins designed to crosslink target cells (i.e. Tumor cells) to effector cells (i.e. T cells or NK cells), triggering cytotoxic killing of the target by the effector. Structurally, bispecific engagers such as bispecific T cell engagers (BiTEs) are typically composed of two distinct single chain variable fragments (scFvs) connected by a protein linker. There are a large number of engagers that harness T cells (BiTEs), but relatively fewer engagers that harness NK cells, in development.

Though expression of CD3e should be sufficient to engage the scFv of BiTEs, it is generally known in the field that CD3e will not traffic to the cell membrane on its own. According to the literature and general understanding in the field, all components of the T cell receptor (TCR) complex, namely TCR and all CD3 subunits, are typically required for cell surface trafficking. The present disclosure identified different strategies to enable stable CD3e expression on the cell surface. Surprisingly, the inventors discovered that contrary to general understanding, cell surface trafficking may be achieved even without all CD3 subunits. The present disclosure provides that a minimal number of CD3 subunits may be sufficient for cell surface trafficking. Furthermore, while the CD3-targeting scFv of BiTEs typically bind to epitopes containing solely CD3e, it has been demonstrated CD3e needs to co-localize with the rest of TCR:CD3 complex in order for the BiTE:CD3e interaction to occur. However, the lack of TCRs on the NK cell surface presents a challenge in utilizing CD3e-targeting BiTEs with NK cells. The present disclosure describes embodiments of expressing CD3e on NK cells for BiTE engagement that overcome this challenge.

Moreover, the engagement of BiTEs to CD3 on a T cell triggers signaling via endogenous ITAMs contained within the TCR:CD3 complex. Therefore, expression of CD3e in NK cells lacking the full TCR:CD3 complex presents a challenge in achieving BiTE-mediated signal transduction. The present disclosure addresses this challenge by incorporating exogenous signaling domains into the engineered CD3e receptor to generate improved intracellular domains.

The disclosure provides engineered NK cells that express CD3e, a T cell antigen typically targeted by BiTEs. The engineered NK cells of the disclosure can be used in combination with clinically approved BiTEs or those undergoing the approval process. The engineered NK cells comprising chimeric receptors that engage bispecific targeting agents may be used in novel indications with target flexibility. Additionally, use of a chimeric receptor with improved intracellular domains would provide signaling selective to NK cells, improving function.

### Chimeric Receptors

In the present disclosure, chimeric receptors (CRs) refer to engineered receptors wherein one or more native structures are substituted, displaced, or otherwise genetically modified. These structures include but are not limited to: the extracellular domain, the transmembrane domain, and the intracellular domain. In some embodiments, these structures can be replaced with multiple other structures. For example, a single native intracellular signaling domain can be substituted with an engineered intracellular domain comprising multiple other intracellular domains. In some other embodiments, the structures can be linked to other peptides, proteins, or domains to generate novel structures. As such, the terms “chimeric receptor” and “chimeric antigen receptor” and “chimeric receptors” and “chimeric antigen receptors” and “CRs” and “CARs” are used interchangeably in the present disclosure to refer to an engineered receptor as described herein.

Chimeric antigen receptors (CARs) are generally designed in a modular fashion that includes at least an extracellular target-binding domain, a transmembrane domain that anchors the CAR to the cell membrane, and one or more intracellular signaling domains (also known as costimulatory domains) that transmit activation signals within the cell. The domains present within a CAR construct are operably linked with suitable linker sequences.

Much of the information known about CARs has come from studies of CAR T cells. However, CAR NK cells have unique benefits in the therapeutic setting. Introduction of a functional CAR molecule into an NK cell can effectively redirect the NK cell with new antigen specificity and can provide the necessary signals to drive full NK cell activation. Also, because antigen recognition by CAR-modified NK cells is based on the binding of the target-binding region of an extracellular domain (e.g., an scFv sequence) to intact surface antigens, targeting of tumor cells is not MHC restricted, co-receptor dependent, or dependent on processing and effective presentation of target epitopes.

As described in the present disclosure, the extracellular domain of a CAR can be the extracellular domain of proteins, such as CD3e or CD28, instead of an antigen-specific scFv. These CARs can be used in combination with bispecific T cell engagers to target novel antigens. In some embodiments, the present disclosure provides a CAR construct comprising at least one extracellular domain, at least one intracellular domain, and a transmembrane domain as specified in rows 1-23 of Table 1.

TABLE 1
Novel CAR constructs

| | Extra- | Extra- | Trans- | |
|---|---|---|---|---|
| Construct | cellular | cellular | membrane | Intracellular |
| # | domain 1 | domain 2 | domain | signaling domain |
| 1 | CD3e | | CD16 | 2B4, CD79a, CD79b, |
| | | | | and CD132 |
| 2 | CD3e | CD3g | CD16 | 2B4, CD79a, CD79b, |
| | | | | and CD132 |
| 3 | CD3e | CD3d | CD16 | 2B4, CD79a, CD79b, |
| | | | | and CD132 |
| 4 | CD3d | CD3e | CD16 | 2B4, CD79a, CD79b, |
| | | | | and CD132 |
| 5 | CD3g | CD3e | CD16 | 2B4, CD79a, CD79b, |
| | | | | and CD132 |
| 6 | CD3e | | CD3e | 2B4, CD79a, CD79b, |
| | | | | and CD132 |
| 7 | CD3e | CD3g | CD3e | 2B4, CD79a, CD79b, |
| | | | | and CD132 |
| 8 | CD3e | CD3d | CD3e | 2B4, CD79a, CD79b, |
| | | | | and CD132 |
| 9 | CD3d | CD3e | CD3e | 2B4, CD79a, CD79b, |
| | | | | and CD132 |
| 10 | CD3g | CD3e | CD3e | 2B4, CD79a, CD79b, |
| | | | | and CD132 |
| 11 | CD3e | | CD3g | 2B4, CD79a, CD79b, |
| | | | | and CD132 |
| 12 | CD3e | CD3g | CD3g | 2B4, CD79a, CD79b, |
| | | | | and CD132 |
| 13 | CD3e | CD3d | CD3g | 2B4, CD79a, CD79b, |
| | | | | and CD132 |
| 14 | CD3d | CD3e | CD3g | 2B4, CD79a, CD79b, |
| | | | | and CD132 |
| 15 | CD3g | CD3e | CD3g | 2B4, CD79a, CD79b, |
| | | | | and CD132 |
| 16 | CD3e | | CD3e | CD132 |
| 17 | CD3e | | CD3e | CD132, 41BB |
| 18 | CD3e | | CD3e | CD132, 41BB, CD79a |
| 19 | CD3g | | CD3g | IL2RB, CD3z ITAM |
| 20 | CD3e | | CD3e | CD3z |
| 21 | CD3g | | CD3g | IL2RB, Fcg ITAM |
| 22 | CD3g | | CD3g | IL2RB, CD79b |
| 23 | CD3g | | CD3g | 41BB |
| 24 | CD3g | CD3e | CD3e | 41BB, CD3z |

The CXXC motif is a highly conserved set of 4 amino acids found on the membrane proximal end of the CD3e, CD3g, and CD3d extracellular domains. This motif is thought to confer some rigidity to the alignment of these extracellular domains to the cell surface. As such, mutating the CXXC motif may improve cell surface expression of a CD3 chimeric receptor. Therefore, in some embodiments, the chimeric receptor constructs of the present disclosure comprise extracellular domains of CD3e, CD3g and CD3d, where the CXXC motif is mutated or removed altogether (truncated).

In some embodiments, the chimeric receptor constructs described in the present disclosure can be encoded by a vector, such as a viral vector (i.e. lentivirus), for expression. In some embodiments, the chimeric receptor is expressed under the control of a promoter that is transcriptionally active in NK cells. In some embodiments, the promoter is MND. In some embodiments, the chimeric receptor comprises a P2A truncated CD34 protein on the terminal end. In some embodiments, the constructs have a CD8α or IgL leader sequence. In some embodiments, the constructs comprise a cleavable linker of any suitable length. In some embodiments, the cleavable linker is P2A. In some embodiments, the chimeric receptor comprises a signal peptide. In some embodiments, the signal peptide is the CD3e, CD3d, or CD3g signal peptide.

Furthermore, illustrative CAR domain sequences are set out below. It will be understood that the present disclosure is not limited to the use of these specific sequences. Rather, it will be understood that these specific sequences can be modified in various ways, including but not limited to insertion, deletion, or substitution of nucleotides, while still improving or retaining a desired level of activity or functionality within an engineered NK cell. As such, in addition to the specific CAR domain sequences described below, the present disclosure also relates to functional fragments and variants of such sequences, e.g., nucleic acid sequences having at least 80%, 85%, 90%, 95% or 99% identity to a specific nucleic acid sequence disclosed herein, as well as to functional fragments and variants comprising amino acid sequences that are at least 80%, 85%, 90%, 95% or 99% identity to an amino acid sequence encoded by a specific CAR domain sequence disclosed herein.

### A. CAR Intracellular Signaling Domains

The disclosure provides CAR intracellular signaling domains that are highly active in NK cells that offer additional unexpected advantages. Specifically, combinations of intracellular signaling domains selected or derived from CD132, CD79A, CD79B, 2B4 and/or DAP10 in NK CAR constructs overcomes many of the limitations associated with other intracellular signaling domains that have been used in NK cells. The combinations of the disclosure overcome these limitations by providing a more potent chimeric receptor compared to those commonly used in CAR T cells and also compared to those derived from endogenous NK cell receptors. As a result, NK cells containing the CAR constructs of the present disclosure are more effective at lower doses and more resilient to tumor immuno-suppression and evasion. In addition, transduction with CARs containing these intracellular signaling domains or other combinations of suitable intracellular signaling domains disclosed herein results in self-enrichment of transduced NK cells during manufacturing and the failure of contaminating T cells to expand as observed when using standard T cell intracellular signaling domains.

In some embodiments, the present disclosure provides a chimeric antigen receptor (CAR) construct capable of being expressed in a natural killer (NK) cell, where the CAR construct comprises a combination of intracellular signaling domains selected or derived from a CD132 intracellular signaling domain, CD79A intracellular signaling domain, a CD79B intracellular signaling domain, a 2B4 intracellular signaling domain and a DAP10 signaling domain.

Illustrative intracellular signaling domain sequences for these and other intracellular domains are set out below.

Illustrative Intracellular Signaling Domain
Sequences:
CD79a_WT
(SEQ ID NO: 1)
CGCAAGCGCTGGCAGAACGAGAAGCTGGGCCTGGACGCCGGCGACGAG
TACGAGGACGAGAACCTGTACGAGGGCCTGAACCTGGACGACTGCTCC
ATGTACGAGGACATCTCCCGCGGCCTGCAGGGCACCTACCAGGACGTG
GGCTCCCTGAACATCGGCGACGTGCAGCTGGAGAAGCCC
CD79a_Mut
(SEQ ID NO: 2)
CGCAAGCGCTGGCAGAACGAGAAGCTGGGCCTGGACGCCGGCGACGAG
TACGAGGACGAGAACCTGTACGAGGGCCTGAACCTGGACGACTGCGCC
ATGTACGAGGACATCGCCCGCGGCCTGCAGGGCGTGTACCAGGACGTG
GGCTCCCTGAACATCGGCGACGTGCAGCTGGAGAAGCCC
CD79b_WT
(SEQ ID NO: 3)
CTGGACAAGGACGACTCCAAGGCCGGCATGGAGGAGGACCACACCTAC
GAGGGCCTGGACATCGACCAGACCGCCACCTACGAGGACATCGTGACC
CTGCGCACCGGCGAGGTGAAGTGGTCCGTGGGCGAGCACCCCGGCCAG
GAG
2B4
(SEQ ID NO: 4)
TGGCGCCGCAAGCGCAAGGAGAAGCAGTCCGAGACCTCCCCCAAGGAG
TTCCTGACCATCTACGAGGACGTGAAGGACCTGAAGACCCGCCGCAAC
CACGAGCAGGAGCAGACCTTCCCCGGCGGCGGCTCCACCATCTACTCC
ATGATCCAGTCCCAGTCCTCCGCCCCCACCTCCCAGGAGCCCGCCTAC
ACCCTGTACTCCCTGATCCAGCCCTCCCGCAAGTCCGGCTCCCGCAAG
CGCAACCACTCCCCCTCCTTCAACTCCACCATCTACGAGGTGATCGGC
AAGTCCCAGCCCAAGGCCCAGAACCCCGCCCGCCTGTCCCGCAAGGAG
CTGGAGAACTTCGACGTGTACTCC
DAP10
(SEQ ID NO: 5)
CTGTGCGCCCGCCCCCGCCGCTCCCCCGCCCAGGAGGACGGCAAGGTG
TACATCAACATGCCCGGCCGCGGC
CD132
(SEQ ID NO: 6)
TTCTGGCTGGAACGGACGATGCCCCGAATTCCCACCCTGAAGAACCTA
GAGGATCTTGTTACTGAATACCACGGGAACTTTTCGGCCTGGAGTGGT
GTGTCTAAGGGACTGGCTGAGAGTCTGCAGCCAGACTACAGTGAACGA
CTCTGCCTCGTCAGTGAGATTCCCCCAAAAGGAGGGGCCCTTGGGGAG
GGGCCTGGGGCCTCCCCATGCAACCAGCATAGCCCCTACTGGGCCCCC
CCATGTTACACCCTAAAGCCTGAAACC
IL2Rb
(SEQ ID NO: 64)
AACTGCCGCAACACCGGCCCCTGGCTGAAGAAGGTGCTGAAGTGCAAC
ACCCCCGACCCCTCCAAGTTCTTCTCCCAGCTGTCCTCCGAGCACGGC
GGCGACGTGCAGAAGTGGCTGTCCTCCCCCTTCCCCTCCTCCTCCTTC
TCCCCCGGCGGCCTGGCCCCCGAGATCTCCCCCCTGGAGGTGCTGGAG
CGCGACAAGGTGACCCAGCTGCTGCTGCAGCAGGACAAGGTGCCCGAG
CCCGCCTCCCTGTCCTCCAACCACTCCCTGACCTCCTGCTTCACCAAC
CAGGGCTACTTCTTCTTCCACCTGCCCGACGCCCTGGAGATCGAGGCC
TGCCAGGTGTACTTCACCTACGACCCCTACTCCGAGGAGGACCCCGAC
GAGGGCGTGGCCGGCGCCCCCACCGGCTCCTCCCCCCAGCCCCTGCAG
CCCCTGTCCGGCGAGGACGACGCCTACTGCACCTTCCCCTCCCGCGAC
GACCTGCTGCTGTTCTCCCCCTCCCTGCTGGGCGGCCCCTCCCCCCCC
TCCACCGCCCCCGGCGGCTCCGGCGCCGGCGAGGAGCGCATGCCCCCC
TCCCTGCAGGAGCGCGTGCCCCGCGACTGGGACCCCCAGCCCCTGGGC
CCCCCCACCCCCGGCGTGCCCGACCTGGTGGACTTCCAGCCCCCCCCC
GAGCTGGTGCTGCGCGAGGCCGGCGAGGAGGTGCCCGACGCCGGCCCC
CGCGAGGGCGTGTCCTTCCCCTGGTCCCGCCCCCCCGGCCAGGGCGAG
TTCCGCGCCCTGAACGCCCGCCTGCCCCTGAACACCGACGCCTACCTG
TCCCTGCAGGAGCTGCAGGGCCAGGACCCCACCCACCTGGTG

In addition to the presence of two or three intracellular signaling domains chosen or derived from a CD132 intracellular signaling domain, a 2B4 intracellular signaling domain, a CD79a intracellular signaling domain and a CD79b intracellular signaling domain, it will be understood that one or more additional intracellular signaling domains may be utilized in a CAR construct of the present disclosure. Illustratively, in some embodiments, such intracellular signaling domain sequences can be chosen or derived from an intracellular signaling domain of CD3 zeta ITAM, CD137/41BB (TRAF, NFkB), DNAM-1 (Y-motif), NKp80 (Y-motif), CRACC (CS1/SLAMF7)::ITSM, CD2 (Y-motifs, MAPK/Erk), CD27 (TRAF, NFkB), or integrins, a cytokine receptor associated with persistence, survival, or metabolism, such as IL-2/15Rbyc:: Jak1/3, STAT3/5, PI3K/mTOR, and MAPK/ERK; a cytokine receptor associated with activation, such as IL-18R::NFkB, a cytokine receptor associated with IFN-γ production, such as IL-12R::STAT4; a cytokine receptor associated with cytotoxicity or persistence, such as IL-21R::Jak3/Tyk2, or STAT3.

CD137/41BB
(SEQ ID NO: 7)
aaacggggcagaaagaaactcctgtatatattcaaacaaccatttatg
agaccagtacaaactactcaagaggaagatggctgtagctgccgattt
ccagaagaagaagaaggaggatgtgaactg
DNAM-1
(SEQ ID NO: 8)
aaggagaaggagagagagaagagatctatttacagagtcctgggatac
acagaaggcacccaataactatagaagtcccatctctaccagtcaacc
taccaatcaatccatggatgatacaagagaggatatttatgtcaacta
tccaaccttctctcgcagaccaaagactagagtttaag
NKp80
(SEQ ID NO: 9)
tttctcagggagtattgctaaaatgccaaaaaggaagttgttcaaatg
ccactcagtatgaggacactggagatctaaaagtgaataatggcacaa
gaagaaatataagtaataaggacctttgtgcttcgagatctgcagacc
agacagtactatgccaatcagaatggctcaaataccaagggaagtgtt
attggttctctaatgagatgaaaagctggagtgacagttatgtgtatt
gtttggaaagaaaatctcatctactaatcatacatgaccaacttgaaa
tggcttttatacagaaaaacctaagacaattaaactacgtatggattg
ggcttaactttacctccttgaaaatgacatggacttgggtggatggtt
ctccaatagattcaaagatattcttcataaagggaccagctaaagaaa
acagctgtgctgccattaaggaaagcaaaattttctctgaaacctgca
gcagtgttttcaaatggatttgtcagtattag
NTBA
(SEQ ID NO: 10)
attccctatctttgtctactcagcgaacacagggccccgcagagtccg
caaggaacctagagtatgtttcagtgtctccaacgaacaacactgtgt
atgcttcagtcactcattcaaacagggaaacagaaatctggacaccta
gagaaaatgatactatcacaatttactccacaattaatcattccaaag
agagtaaacccactttttccagggcaactgcccttgacaatgtcgtgt
aa
CRACC
(SEQ ID NO: 11)
agtacattgaagagaagaagagagtggacatttgtcgggaaactccta
acatatgcccccattctggagagaacacagagtacgacacaatccctc
acactaatagaacaatcctaaaggaagatccagcaaatacggtttact
ccactgtggaaataccgaaaaag
CD2
(SEQ ID NO: 12)
aaaaggaaaaaacagaggagtcggagaaatgatgaggagctggagaca
agagcccacagagtagctactgaagaaaggggccggaagccccaccaa
attccagcttcaacccctcagaatccagcaacttcccaacatcctcct
ccaccacctggtcatcgttcccaggcacctagtcatcgtcccccgcct
cctggacaccgtgttcagcaccagcctcagaagaggcctcctgctccg
tcgggcacacaagttcaccagcagaaaggcccgcccctccccagacct
cgagttcagccaaaacctccccatggggcagcagaaaactcattgtcc
ccttcctctaattaa
CD27
(SEQ ID NO: 13)
Atccttgtgatcttctctggaatgttccttgttttcaccctggccggg
gccctgttcctccatcaacgaaggaaatatagatcaaacaaaggagaa
agtcctgtggagcctgcagagccttgtcgttacagctgccccagggag
gaggagggcagcaccatccccatccaggaggattaccgaaaaccggag
cctgcctgctccccctga
OX40
(SEQ ID NO: 76)
GCCCTGTACCTGCTGCGCCGCGACCAGCGCCTGCCCCCCGACGCCCAC
AAGCCCCCCGGCGGCGGCTCCTTCCGCACCCCCATCCAGGAGGAGCAG
GCCGACGCCCACTCCACCCTGGCCAAGATC
Integrins
A. ITGB1
(SEQ ID NO: 14)
aagcttttaatgataattcatgacagaagggagtttgctaaatttgaa
aaggagaaaatgaatgccaaatgggacacgggtgaaaatcctatttat
aagagtgccgtaacaactgtggtcaatccgaagtatgagggaaaatga
B. ITGB2
(SEQ ID NO: 15)
aaggctctgatccacctgagcgacctccgggagtacaggcgctttgag
aaggagaagctcaagtcccagtggaacaatgataatccccttttcaag
agcgccaccacgacggtcatgaaccccaagtttgctgagagttag
C. ITGB3
(SEQ ID NO: 16)
aaactcctcatcaccatccacgaccgaaaagaattcgctaaatttgag
gaagaacgcgccagagcaaaatgggacacagccaacaacccactgtat
aaagaggccacgtctaccttcaccaatatcacgtaccggggcacttaa
IL15RB
(SEQ ID NO: 17)
aactgcaggaacaccgggccatggctgaagaaggtcctgaagtgtaac
accccagacccctcgaagttcttttcccagctgagctcagagcatgga
ggagacgtccagaagtggctctcttcgcccttcccctcatcgtccttc
agccctggcggcctggcacctgagatctcgccactagaagtgctggag
agggacaaggtgacgcagctgctcctgcagcaggacaaggtgcctgag
cccgcatccttaagcagcaaccactcgctgaccagctgcttcaccaac
cagggttacttcttcttccacctcccggatgccttggagatagaggcc
tgccaggtgtactttacttacgacccctactcagaggaagaccctgat
gagggtgtggccggggcacccacagggtcttccccccaacccctgcag
cctctgtcaggggaggacgacgcctactgcaccttcccctccagggat
gacctgctgctcttctcccccagtctcctcggtggccccagcccccca
agcactgcccctgggggcagtggggccggtgaagagaggatgccccct
tctttgcaagaaagagtccccagagactgggacccccagcccctgggg
cctcccaccccaggagtcccagacctggtggattttcagccaccccct
gagctggtgctgcgagaggctggggaggaggtccctgacgctggcccc
agggagggagtcagtttcccctggtccaggcctcctgggcagggggag
ttcagggcccttaatgctcgcctgcccctgaacactgatgcctacttg
tccctccaagaactccagggtcaggacccaactcacttggtgtag
IL18R
(SEQ ID NO: 18)
tataaagttgacttggttctgttctataggcgcatagcggaaagagac
gagacactaacagatggtaaaacatatgatgcctttgtgtcttacctg
aaagagtgtcatcctgagaataaagaagagtatacttttgctgtggag
acgttacccagggtcctggagaaacagtttgggtataagttatgcata
tttgaaagagatgtggtgcctggcggagctgttgtcgaggagatccat
tcactgatagagaaaagccggaggctaatcatcgttctcagccagagt
tacctgactaacggagccaggcgtgagctcgagagtggactccacgaa
gcactggtagagaggaagattaagatcatcttaattgagtttactcca
gccagcaacatcacctttctccccccgtcgctgaaactcctgaagtcc
tacagagttctaaaatggagggctgacagtccctccatgaactcaagg
ttctggaagaatcttgtttacctgatgcccgcaaaagccgtcaagcca
tggagagaggagtcggaggcgcggtctgttctctcagcaccttga
IL12R
IL12RB1
(SEQ ID NO: 119)
aacagggccgcacggcacctgtgcccgccgctgcccacaccctgtgcc
agctccgccattgagttccctggagggaaggagacttggcagtggatc
aacccagtggacttccaggaagaggcatccctgcaggaggccctggtg
gtagagatgtcctgggacaaaggcgagaggactgagcctctcgagaag
acagagctacctgagggtgcccctgagctggccctggatacagagttg
tccttggaggatggagacaggtgcaaggccaagatgtga
IL12RB2
(SEQ ID NO: 20)
cattacttccagcaaaaggtgtttgttctcctagcagccctcagacct
cagtggtgtagcagagaaattccagatccagcaaatagcacttgcgct
aagaaatatcccattgcagaggagaagacacagctgcccttggacagg
ctcctgatagactggcccacgcctgaagatcctgaaccgctggtcatc
agtgaagtccttcatcaagtgaccccagttttcagacatcccccctgc
tccaactggccacaaagggaaaaaggaatccaaggtcatcaggcctct
gagaaagacatgatgcacagtgcctcaagcccaccacctccaagagct
ctccaagctgagagcagacaactggtggatctgtacaaggtgctggag
agcaggggctccgacccaaagcccgaaaacccagcctgtccctggacg
gtgctcccagcaggtgaccttcccacccatgatggctacttaccctcc
aacatagatgacctcccctcacatgaggcacctctcgctgactctctg
gaagaactggagcctcagcacatctccctttctgttttcccctcaagt
tctcttcacccactcaccttctcctgtggtgataagctgactctggat
cagttaaagatgaggtgtgactccctcatgctctga
IL21R
(SEQ ID NO: 21)
agcctgaagacccatccattgtggaggctatggaagaagatatgggcc
gtccccagccctgagcggttcttcatgcccctgtacaagggctgcagc
ggagacttcaagaaatggggggtgcacccttcactggctccagcctgg
agctgggaccctggagcccagaggtgccctccaccctggaggtgtaca
gctgccacccaccacggagcccggccaagaggctgcagctcacggagc
tacaagaaccagcagagctggtggagtctgacggtgtgcccaagccca
gcttctggccgacagcccagaactcggggggctcagcttacagtgagg
agagggatcggccatacggcctggtgtccattgacacagtgactgtgc
tagatgcagaggggccatgcacctggccctgcagctgtgaggatgacg
gctacccagccctggacctggatgctggcctggagcccagcccaggcc
tagaggacccactcttggatgcagggaccacagtcctgtcctgtggct
gtgtctcagctggcagccctgggctaggagggcccctgggaagcctcc
tggacagactaaagccaccccttgcagatggggaggactgggctgggg
gactgccctggggtggccggtcacctggaggggtctcagagagtgagg
cgggctcacccctggccggcctggatatggacacgtttgacagtggct
ttgtgggctctgactgcagcagccctgtggagtgtgacttcaccagcc
ccggggacgaaggacccccccggagctacctccgccagtgggtggtca
ttcctccgccactttcgagccctggaccccaggccagctaa
IRE1a
(SEQ ID NO: 22)
cccctgagcatgcatcagcagcagcagctccagcaccagcagttccag
aaggaactggagaagatccagctcctgcagcagcagcagcagcagctg
cccttccacccacctggagacacggctcaggacggcgagctcctggac
acgtctggcccgtactcagagagctcgggcaccagcagccccagcacg
tcccccagggcctccaaccactcgctctgctccggcagctctgcctcc
aaggctggcagcagcccctccctggaacaagacgatggagatgaggaa
accagcgtggtgatagttgggaaaatttccttctgtcccaaggatgtc
ctgggccatggagctgagggcacaattgtgtaccggggcatgtttgac
aaccgcgacgtggccgtgaagaggatcctccccgagtgttttagcttc
gcagaccgtgaggtccagctgttgcgagaatcggatgagcacccgaac
gtgatccgctacttctgcacggagaaggaccggcaattccagtacatt
gccatcgagctgtgtgcagccaccctgcaagagtatgtggagcagaag
gactttgcgcatctcggcctggagcccatcaccttgctgcagcagacc
acctcgggcctggcccacctccactccctcaacatcgttcacagagac
ctaaagccacacaacatcctcatatccatgcccaatgcacacggcaag
atcaaggccatgatctccgactttggcctctgcaagaagctggcagtg
ggcagacacagtttcagccgccgatctggggtgcctggcacagaaggc
tggatcgctccagagatgctgagcgaagactgtaaggagaaccctacc
tacacggtggacatcttttctgcaggctgcgtcttttactacgtaatc
tctgagggcagccacccttttggcaagtccctgcagcggcaggccaac
atcctcctgggtgcctgcagccttgactgcttgcacccagagaagcac
gaagacgtcattgcacgtgaattgatagagaagatgattgcgatggat
cctcagaaacgcccctcagcgaagcatgtgctcaaacacccgttcttc
tggagcctagagaagcagctccagttcttccaggacgtgagcgacaga
atagaaaaggaatccctggatggcccgatcgtgaagcagttagagaga
ggcgggagagccgtggtgaagatggactggcgggagaacatcactgtc
cccctccagacagacctgcgtaaattcaggacctataaaggtggttct
gtcagagatctcctccgagccatgagaaataagaagcaccactaccgg
gagctgcctgcagaggtgcgggagacgctggggtccctccccgacgac
ttcgtgtgctacttcacatctcgcttcccccacctcctcgcacacacc
taccgggccatggagctgtgcagccacgagagactcttccagccctac
tacttccacgagcccccagagccccagcccccagtgactccagacgcc
ctctga

### B. CAR Extracellular Domains (ECD)

The CAR constructs of the present disclosure generally include an extracellular domain. In some embodiments, the extracellular domain is capable of binding to a clinical antibody of interest that binds more than one target of interest, including an antigen of interest, such as an antigen associated with an infectious disease, a bacterial infection, a virus, a cancer, an autoimmune disease, or an immune disorder or dysfunction. In some embodiments, the extracellular domain binds to a T cell engager. In some embodiments, the T cell engager is a bispecific T cell engager (BiTE) of interest where one fragment antigen-binding region Fab targets CD3e or CD28 and the second antigen-binding Fab targets an antigen associated with an infectious disease, a bacterial infection, a virus, a cancer, an autoimmune disease, or an immune disorder or dysfunction. In some embodiments, the T cell engager is a trispecific T cell engager. In some embodiments, the T cell engager is a quadrispecific T cell engager. In some embodiments, the trispecific or quadrispecific T cell engager has at least one antigen-binding region that targets CD3e or CD28.

Cell surface CD3e expression can also be improved or stabilized through covalent linkage to other CD3 subunits. Therefore, in some embodiments, the extracellular domain of CAR can be a combination of the ECD of CD3e, covalently linked to the ECD of CD3g or the ECD of CD3d. In some embodiments, these ECDs are linked with a linker of any suitable length that preserves the function of the subunits.

Illustrative sequences of a CD3e extracellular domain and a CD28 extracellular domain capable of binding BiTEs are provided below.

CD3e ECD
(SEQ ID NO: 23)
ATGGCCTTACCAGTGACCGCCTTGCTCCTGCCGCTGGCCTTGCTGCTC
CACGCCGCCAGGCCGGACGGCAACGAGGAGATGGGCGGCATCACCCAG
ACCCCCTACAAGGTGTCCATCTCCGGCACCACCGTGATCCTGACCTGC
CCCCAGTACCCCGGCTCCGAGATCCTGTGGCAGCACAACGACAAGAAC
ATCGGCGGCGACGAGGACGACAAGAACATCGGCTCCGACGAGGACCAC
CTGTCCCTGAAGGAGTTCTCCGAGCTGGAGCAGTCCGGCTACTACGTG
TGCTACCCCCGCGGCTCCAAGCCCGAGGACGCCAACTTCTACCTGTAC
CTGCGCGCCCGCGTGTGCGAGAACTGCATGGAGATGGAC
CD28 ECD
(SEQ ID NO: 24)
ATGGCCTTACCAGTGACCGCCTTGCTCCTGCCGCTGGCCTTGCTGCTC
CACGCCGCCAGGCCGAACAAGATCCTGGTGAAGCAGTCCCCCATGCTG
GTGGCCTACGACAACGCCGTGAACCTGTCCTGCAAGTACTCCTACAAC
CTGTTCTCCCGCGAGTTCCGCGCCTCCCTGCACAAGGGCCTGGACTCC
GCCGTGGAGGTGTGCGTGGTGTACGGCAACTACTCCCAGCAGCTGCAG
GTGTACTCCAAGACCGGCTTCAACTGCGACGGCAAGCTGGGCAACGAG
TCCGTGACCTTCTACCTGCAGAACCTGTACGTGAACCAGACCGACATC
TACTTCTGCAAGATCGAGGTGATGTACCCCCCCCCCTACCTGGACAAC
GAGAAGTCCAACGGCACCATCATCCACGTGAAGGGCAAGCACCTGTGC
CCCTCCCCCCTGTTCCCCGGCCCCTCCAAGCCCTTC
CD3G ECD
(SEQ ID NO: 65)
ATGGAGCAGGGCAAGGGCCTGGCCGTGCTGATCCTGGCCATCATCCTG
CTGCAGGGCACCCTGGCCCAGTCCATCAAGGGCAACCACCTGGTGAAG
GTGTACGACTACCAGGAGGACGGCTCCGTGCTGCTGACCTGCGACGCC
GAGGCCAAGAACATCACCTGGTTCAAGGACGGCAAGATGATCGGCTTC
CTGACCGAGGACAAGAAGAAGTGGAACCTGGGCTCCAACGCCAAGGAC
CCCCGCGGCATGTACCAGTGCAAGGGCTCCCAGAACAAGTCCAAGCCC
CTGCAGGTGTACTACCGCATGTGCCAGAACTGCATCGAGCTGAACGCC
GCCACCATCTCC
CD3D ECD
(SEQ ID NO: 66)
ATGGAGCACTCCACCTTCCTGTCCGGCCTGGTGCTGGCCACCCTGCTG
TCCCAGGTGTCCCCCTTCAAGATCCCCATCGAGGAGCTGGAGGACCGC
GTGTTCGTGAACTGCAACACCTCCATCACCTGGGTGGAGGGCACCGTG
GGCACCCTGCTGTCCGACATCACCCGCCTGGACCTGGGCAAGCGCATC
CTGGACCCCCGCGGCATCTACCGCTGCAACGGCACCGACATCTACAAG
GACAAGGAGTCCACCGTGCAGGTGCACTACCGCATGTGCCAGTCCTGC
GTGGAGCTGGACCCCGCCACCGTGGCC

In some embodiments, the extracellular domain is capable of binding to a target polypeptide of interest, such as an antigen associated with an infectious disease, a bacterial infection, a virus, a cancer, an autoimmune disease, or an immune disorder or dysfunction.

In some embodiments, the extracellular domain of a CAR construct of the present disclosure comprises an antibody fragment. For example, in some embodiments, the extracellular domain of a CAR construct of the present disclosure comprises a single-chain variable fragment sequence (scFv sequence) capable of binding a target polypeptide of interest, such as a disease-associated target polypeptide.

scFvs are well known in the art to be used as a binding moiety in a variety of constructs (see e.g., Sentman 2014 Cancer J. 20 156-159; Guedan 2019 Mol Ther Methods Clin Dev. 12 145-156). scFvs can be against any antigen known in the art, such as those described in US20160361360A1, which is incorporated herein by reference in its entirety. Any scFv known in the art or generated against an antigen using means known in the art can be used as the binding moiety in an extracellular domain of a CAR construct of the present disclosure.

The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the NK cell surface or whether the NK cells target the antigen and signal. In addition, the length and/or composition of the variable domain linker can contribute to the stability or affinity of the scFv.

CAR scFv affinities, modified through mutagenesis of complementary-determining regions while holding the epitope constant, or through CAR development with scFvs derived from therapeutic antibodies against the same target, but not the same epitope, can change the strength of the NK cell signal and allow NK cells to differentiate overexpressed antigens from normally expressed antigens. The scFv, a critical component of a CAR molecule, can be carefully designed and manipulated to influence specificity and differential targeting of tumors versus normal tissues. Given that these differences may only be measurable with NK cells (as opposed to soluble antibodies), pre-clinical testing of normal tissues for expression of the target and susceptibility to on-target toxicities typically requires live-cell assays rather than immunohistochemistry on fixed tissues.

In some embodiments, the scFvs described herein can be used for hematological malignancies such as AML, ALL, or Lymphoma, but can also be expanded for use in any malignancy, autoimmune, or infectious disease where a scFv can be generated against a target antigen or antigen epitope. For example, the constructs described herein can be used to treat or prevent autoimmunity associated with auto-antibodies (similar indications as rituximab for autoimmunity). As another example, the disclosed constructs can also be applied to virally infected cells, using scFv that can recognize viral antigens, for example gp120 and gp41 on HIV-infected cells.

Examples of disease-associated polypeptides that can be advantageously targeted by the CAR extracellular domains according to the disclosure can include any target. In some embodiments, the polypeptide targeted and bound by the extracellular domain of the CAR is selected from the group consisting of CD2, CD5, CD7, MSLN, CEA, PSMA, CD19, CD28, CD3, CD33, CD38, CD138, CLL-1, CLL-3, C-KIT CD123, CD133, CD20, BCMA, EGFR, CD3, CD4, BAFF-R, EGFR, HER2, GD2, gp120 and gp41.

Illustrative scFv sequences capable of binding some of these target polypeptides are provided below.

Anti-Mesothelin scFv
(SEQ ID NO: 25)
ATGGCCTTACCAGTGACCGCCTTGCTCCTGCCGCTGGCCTTGCTGCTC
CACGCCGCCAGGCCGGGATCCCAGGTACAACTGCAGCAGTCTGGGCCT
GAGCTGGAGAAGCCTGGCGCTTCAGTGAAGATATCCTGCAAGGCTTCT
GGTTACTCATTCACTGGCTACACCATGAACTGGGTGAAGCAGAGCCAT
GGAAAGAGCCTTGAGTGGATTGGACTTATTACTCCTTACAATGGTGCT
TCTAGCTACAACCAGAAGTTCAGGGGCAAGGCCACATTAACTGTAGAC
AAGTCATCCAGCACAGCCTACATGGACCTCCTCAGTCTGACATCTGAA
GACTCTGCAGTCTATTTCTGTGCAAGGGGGGGTTACGACGGGAGGGGT
TTTGACTACTGGGGCCAAGGGACCACGGTCACCGTCTCCTCAGGTGGA
GGCGGTTCAGGCGGCGGTGGCTCTAGCGGTGGTGGATCGGACATCGAG
CTCACTCAGTCTCCAGCAATCATGTCTGCATCTCCAGGGGAGAAGGTC
ACCATGACCTGCAGTGCCAGCTCAAGTGTAAGTTACATGCACTGGTAC
CAGCAGAAGTCAGGCACCTCCCCCAAAAGATGGATTTATGACACATCC
AAACTGGCTTCTGGAGTCCCAGGTCGCTTCAGTGGCAGTGGGTCTGGA
AACTCTTACTCTCTCACAATCAGCAGCGTGGAGGCTGAAGATGATGCA
ACTTATTACTGCCAGCAGTGGAGTAAGCACCCTCTCACGTACGGTGCT
GGGACAAAGTTGGAAATCAAAGCTAGC
Anti-CD19 scFv
(SEQ ID NO: 26)
ATGGCCTTACCAGTGACCGCCTTGCTCCTGCCGCTGGCCTTGCTGCTC
CACGCCGCCAGGCCGGACATCCAGATGACACAAACCACCTCCAGCCTG
TCCGCATCCCTTGGAGATCGAGTCACCATTAGCTGCCGGGCGTCCCAG
GACATCAGCAAATACCTCAATTGGTATCAGCAAAAACCCGACGGCACG
GTGAAGCTTCTGATATACCACACTTCACGGCTGCACTCAGGCGTGCCA
AGTCGGTTTTCAGGATCCGGCTCCGGCACCGATTATTCTCTGACCATT
AGCAATCTCGAGCAAGAGGATATCGCCACTTATTTCTGTCAGCAGGGG
AACACCCTCCCTTATACGTTTGGTGGTGGGACCAAGTTGGAGATTACA
CGGGCAGATGCCGCCCCTACTGTGAGCATATTTCCTCCTAGCTCTAAT
GGGGGTGGAGGCTCCGGTGGTGGGGGGAGCGGCGGCGGTGGCTCAGGA
GGCGGCGGCAGCGAGGTCAAACTGCAAGAGTCTGGTCCAGGCCTGGTG
GCCCCCTCCCAGTCTCTCAGTGTGACCTGTACCGTTTCAGGCGTGTCT
TTGCCTGATTACGGCGTGTCCTGGATAAGGCAGCCACCAAGAAAGGGA
CTGGAATGGCTGGGCGTTATCTGGGGATCAGAGACAACTTATTACAAC
TCAGCTCTTAAATCCAGACTCACGATCATTAAGGATAACTCTAAATCC
CAGGTGTTCCTGAAGATGAATTCTCTCCAGACAGACGATACTGCCATC
TACTATTGCGCTAAGCATTATTATTACGGGGGCTCATACGCCATGGAC
TACTGGGGACAGGGCACCAGCGTGACTGTGAGTTCCCCTAGGGCTAGC
Anti-CD19 scFv
(SEQ ID NO: 27)
ATGGCCCTGCCCGTGACCGCTCTCCTGCTGCCTCTGGCCCTGCTCCTC
CATGCTGCCAGACCCGACATCCAGATGACACAGACAACCAGCAGCCTG
TCCGCTTCCCTCGGAGACAGGGTGACAATTTCCTGCAGGGCCAGCCAG
GACATCAGCAAGTACCTGAACTGGTACCAGCAGAAACCCGACGGCACC
GTCAAGCTCCTGATCTACCACACCAGCAGACTGCACAGCGGAGTGCCT
TCCAGGTTCAGCGGCAGCGGCTCCGGCACCGATTACTCCCTGACCATT
AGCAACTTAGAACAGGAGGACATTGCCACCTACTTTTGTCAGCAGGGC
AACACCCTCCCCTACACCTTTGGAGGCGGAACCAAGTTAGAAATCACC
GGCGGCGGCGGCAGCGGAGGAGGAGGCAGCGGAGGCGGAGGCTCCGAG
GTGAAACTGCAGGAGAGCGGCCCCGGACTGGTCGCCCCTAGCCAATCC
CTCTCCGTCACCTGCACCGTGAGCGGAGTGAGCCTGCCTGACTACGGA
GTGAGCTGGATCAGACAGCCCCCTAGGAAAGGACTGGAATGGCTGGGC
GTGATTTGGGGCAGCGAGACCACCTATTACAACAGCGCCCTGAAGTCC
AGACTGACAATCATCAAGGACAATAGCAAAAGCCAAGTGTTTCTGAAG
ATGAACAGCCTGCAGACCGATGACACCGCCATCTATTATTGCGCCAAG
CACTACTACTACGGAGGAAGCTACGCTATGGATTATTGGGGCCAAGGC
ACAAGCGTGACCGTCAGCAGCGCGGCCGCC
Anti-CD33 scFv
(SEQ ID NO: 28)
ATGGCCTTACCAGTGACCGCCTTGCTCCTGCCGCTGGCCTTGCTGCTC
CACGCCGCCAGGCCGATGGAAAAGGATACACTGTTGTTGTGGGTTCTG
CTCCTGTGGGTGCCCGGCAGCACCGGAGATATTGTGCTGACGCAGTCT
CCTGCATCACTCGCCGTGTCTCTGGGCCAGCGCGCTACCATCAGCTGC
AGAGCCTCTGAAAGTGTTGACAATTATGGAATTTCTTTCATGAATTGG
TTCCAGCAGAAGCCTGGCCAGCCCCCGAAACTCCTCATATATGCCGCG
TCTAATCAGGGCTCTGGGGTCCCTGCTAGATTTTCTGGCAGCGGCTCC
GGCACCGACTTCAGTCTGAATATACATCCCATGGAAGAAGACGATACC
GCCATGTACTTTTGCCAACAATCTAAGGAGGTGCCTTGGACGTTCGGC
GGCGGTACGAAGCTGGAAATTAAGGGCGGCGGGGGAAGCGGCGGGGGG
GGATCAGGCGGGGGTGGCTCCGGAGGCGGTGGAAGTATGGGCTGGAGT
TGGATCTTCCTTTTCCTTCTTTCTGGTACCGCGGGAGTGCACTCTGAG
GTGCAGCTCCAGCAGTCCGGCCCCGAGCTCGTCAAGCCTGGGGCCAGT
GTCAAGATTTCCTGTAAGGCATCTGGATATACCTTTACAGATTACAAT
ATGCATTGGGTGAAACAGTCACATGGAAAGTCACTCGAGTGGATCGGA
TACATTTACCCTTACAATGGAGGAACCGGATATAATCAGAAGTTTAAG
AGCAAGGCCACACTCACGGTGGACAATTCTTCATCTACAGCCTACATG
GATGTTCGGTCTCTGACTTCCGAGGATAGTGCGGTGTATTACTGCGCC
AGGGGACGCCCCGCTATGGATTACTGGGGGCAGGGAACCTCTGTAACA
GTTAGCTCA
Anti-CD123 scFv
(SEQ ID NO: 29)
ATGGCCTTACCAGTGACCGCCTTGCTCCTGCCGCTGGCCTTGCTGCTC
CACGCCGCCAGGCCGGACTTCGTGATGACTCAGTCTCCTAGCTCCCTG
ACCGTGACAGCCGGCGAGAAGGTGACCATGTCCTGCAAATCTAGTCAG
AGTCTGCTGAACTCAGGCAATCAGAAGAACTATCTGACATGGTACCTG
CAGAAGCCAGGGCAGCCCCCTAAACTGCTGATCTATTGGGCCAGCACC
AGGGAATCCGGCGTGCCCGACAGATTCACCGGCTCCGGGTCTGGAACA
GATTTTACTCTGACCATTTCAAGCGTGCAGGCCGAGGACCTGGCTGTG
TACTATTGTCAGAATGATTACAGCTATCCCTACACATTTGGCGGGGGA
ACTAAGCTGGAAATCAAAGGTGGTGGTGGTTCTGGTGGTGGTGGTTCC
GGCGGCGGCGGCTCCGGTGGTGGTGGATCCGAGGTGCAGCTGCAGCAG
AGTGGACCCGAACTGGTGAAACCTGGCGCCTCCGTGAAAATGTCTTGC
AAGGCTAGTGGGTACACCTTCACAGACTACTATATGAAATGGGTGAAG
CAGTCACACGGGAAGAGCCTGGAGTGGATCGGAGATATCATTCCCTCT
AACGGCGCCACTTTCTACAATCAGAAGTTTAAAGGCAAGGCTACTCTG
ACCGTGGACCGGAGCTCCTCTACCGCCTATATGCACCTGAACAGTCTG
ACATCAGAAGATAGCGCTGTGTACTATTGTACACGGTCCCATCTGCTG
AGAGCCTCTTGGTTTGCTTATTGGGGCCAGGGGACACTGGTGACTGTG
AGCTCCGCTAGCACCACGACGCCAGCGCCGCGACCACCAACACCGGCG
CCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGT
GATTTTTGGGTGCTGGTGGTGGTTGGTGGAGTCCTGGCTTGCTATAGC
TTGCTAGTAACAGTGGCCTTTATTATTTTCTGGGTG

### Other CAR Extracellular Domains

In some embodiments of the present disclosure, the extracellular domain of a CAR is one that is derived from a cellular receptor, such as an Fc receptor, such as CD16, CD32, CD64 or others. Extracellular domains comprising such sequences are particularly useful in the production of ADCC-enabled NK cells containing a CAR construct of the disclosure. In some embodiments, the CD16 sequence has a mutation corresponding to S197P at the ADAM17 cleavage site in order inhibit cleavage and shedding of the expressed CAR to enhance NK cell activity. As described herein, such Enhanced (E)-ADCC-enabled NK cells exhibit increased cytotoxicity towards different cancer cell types when compared to control NK cells. Additionally, similarly to the other CAR constructs encoding or expressing the intracellular signaling domains described herein, self-enrichment of the CAR-expressing NK cells was observed during the manufacturing process.

Illustrative CD16, CD32 and CD64 sequences capable of being used in the present disclosure.

CD16_S197P ECD-w/mutation at ADAM17 cleavage site
(SEQ ID NO: 30)
ATGGCCTTACCAGTGACCGCCTTGCTCCTGCCGCTGGCCTTGCTGCTCC
ACGCCGCCAGGCCGGGCATGCGCACCGAGGACCTGCCCAAGGCCGTGGT
GTTCCTGGAGCCCCAGTGGTACCGCGTGCTGGAGAAGGACTCCGTGACC
CTGAAGTGCCAGGGCGCCTACTCCCCCGAGGACAACTCCACCCAGTGGT
TCCACAACGAGTCCCTGATCTCCTCCCAGGCCTCCTCCTACTTCATCGA
CGCCGCCACCGTGGACGACTCCGGCGAGTACCGCTGCCAGACCAACCTG
TCCACCCTGTCCGACCCCGTGCAGCTGGAGGTGCACATCGGCTGGCTGC
TGCTGCAGGCCCCCCGCTGGGTGTTCAAGGAGGAGGACCCCATCCACCT
GCGCTGCCACTCCTGGAAGAACACCGCCCTGCACAAGGTGACCTACCTG
CAGAACGGCAAGGGCCGCAAGTACTTCCACCACAACTCCGACTTCTACA
TCCCCAAGGCCACCCTGAAGGACTCCGGCTCCTACTTCTGCCGCGGCCT
GTTCGGCTCCAAGAACGTGTCCTCCGAGACCGTGAACATCACCATCACC
CAGGGCCTGGCCGTGCCCACCATCTCCTCCTTCTTCCCCCCCGGCTACC
AG
CD32 ECD
(SEQ ID NO: 31)
ATGGCCTTACCAGTGACCGCCTTGCTCCTGCCGCTGGCCTTGCTGCTCC
ACGCCGCCAGGCCGCAGGCCGCCGCCCCCCCCAAGGCCGTGCTGAAGCT
GGAGCCCCCCTGGATCAACGTGCTGCAGGAGGACTCCGTGACCCTGACC
TGCCAGGGCGCCCGCTCCCCCGAGTCCGACTCCATCCAGTGGTTCCACA
ACGGCAACCTGATCCCCACCCACACCCAGCCCTCCTACCGCTTCAAGGC
CAACAACAACGACTCCGGCGAGTACACCTGCCAGACCGGCCAGACCTCC
CTGTCCGACCCCGTGCACCTGACCGTGCTGTCCGAGTGGCTGGTGCTGC
AGACCCCCCACCTGGAGTTCCAGGAGGGCGAGACCATCATGCTGCGCTG
CCACTCCTGGAAGGACAAGCCCCTGGTGAAGGTGACCTTCTTCCAGAAC
GGCAAGTCCCAGAAGTTCTCCCACCTGGACCCCACCTTCTCCATCCCCC
AGGCCAACCACTCCCACTCCGGCGACTACCACTGCACCGGCAACATCGG
CTACACCCTGTTCTCCTCCAAGCCCGTGACCATCACCGTGCAGGTGCCC
TCCATGGGCTCCTCCTCCCCCATGGGC
CD64 ECD
(SEQ ID NO: 32)
ATGGCCTTACCAGTGACCGCCTTGCTCCTGCCGCTGGCCTTGCTGCTCC
ACGCCGCCAGGCCGCAGGTGGACACCACCAAGGCCGTGATCACCCTGCA
GCCCCCCTGGGTGTCCGTGTTCCAGGAGGAGACCGTGACCCTGCACTGC
GAGGTGCTGCACCTGCCCGGCTCCTCCTCCACCCAGTGGTTCCTGAACG
GCACCGCCACCCAGACCTCCACCCCCTCCTACCGCATCACCTCCGCCTC
CGTGAACGACTCCGGCGAGTACCGCTGCCAGCGCGGCCTGTCCGGCCGC
TCCGACCCCATCCAGCTGGAGATCCACCGCGGCTGGCTGCTGCTGCAGG
TGTCCTCCCGCGTGTTCACCGAGGGCGAGCCCCTGGCCCTGCGCTGCCA
CGCCTGGAAGGACAAGCTGGTGTACAACGTGCTGTACTACCGCAACGGC
AAGGCCTTCAAGTTCTTCCACTGGAACTCCAACCTGACCATCCTGAAGA
CCAACATCTCCCACAACGGCACCTACCACTGCTCCGGCATGGGCAAGCA
CCGCTACACCTCCGCCGGCATCTCCGTGACCGTGAAGGAGCTGTTCCCC
GCCCCCGTGCTGAACGCCTCCGTGACCTCCCCCCTGCTGGAGGGCAACC
TGGTGACCCTGTCCTGCGAGACCAAGCTGCTGCTGCAGCGCCCCGGCCT
GCAGCTGTACTTCTCCTTCTACATGGGCTCCAAGACCCTGCGCGGCCGC
AACACCTCCTCCGAGTACCAGATCCTGACCGCCCGCCGCGAGGACTCCG
GCCTGTACTGGTGCGAGGCCGCCACCGAGGACGGCAACGTGCTGAAGCG
CTCCCCCGAGCTGGAGCTGCAGGTGCTGGGCCTGCAGCTGCCCACCCCC
GTGTGGTTCCAC

### C. CAR Transmembrane TM Domains and Adapters

The CAR constructs described herein also include a transmembrane (TM) domain comprising a hydrophobic a helix that spans the cell membrane. Although the main function of the transmembrane is to anchor the CAR in the NK cell membrane, some evidence suggests that the transmembrane domain can be relevant for CAR cell function.

The TM domain can be any TM domain suitable that is effective in an NK cell. For example, in some embodiments, the TM domain can be chosen or derived from a transmembrane sequence from CD3e, CD3g, CD3d, CD16, NKG2D, FcγRIIIa, NKp44, NKp30, NKp46, activating KIR (actKIR), NKG2C, or CD8α.

NK cells express transmembrane (TM) adapters (e.g. DAP10, CD3z) that transmit activation signals upon association with activating receptors (e.g. CD16, NKG2D). This provides an NK cell-specific signal enhancement via engineering the TM domains from activating receptors, and thereby harness endogenous adapters. The TM adapter can be any endogenous TM adapter capable of signaling activation. For example, the TM adapter can be FceR1γ (ITAMx1), CD3ζ (ITAMx3), DAP12 (ITAMx1), or DAP10 (YxxM/YINM).

In certain embodiments, the TM domains and adapters are paired, for example, NKG2D and DAP10, FcγRIIIa and CD3ζ or FceR1γ, NKp44 and DAP12, NKp30 and CD3ζ or FceR1γ, NKp46 and CD3ζ or FceR1γ, actKIR and DAP12, and NKG2C and DAP12.

### D. Hinge (Spacer) Domains

The hinge, also referred to as a spacer, is in the extracellular structural region of the CAR that separates the binding units from the transmembrane domain. The hinge can be any moiety capable of ensuring proximity of the CAR NK cell to the target (e.g., NKG2-based hinge, TMα-based hinge, CD8-based hinge). With the exception of a few CARs based on the entire extracellular moiety of a receptor, such as NKG2D, as described herein, the majority of CAR (such as CAR T) cells are designed with immunoglobulin (Ig)-like domain hinges.

Hinges generally supply stability for efficient CAR expression and activity. The NKG2 hinge (also in combination with the transmembrane domain), described herein also ensures proper proximity to target.

The hinge also provides flexibility to access the targeted antigen. An optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly.

Below are illustrative hinge and TM domain sequences. It will be understood that the hinge and TM sequences described below can be mixed, matched, and altered for improved performance.

Hinge/Transmembrane (TM) Domain Sequences
NKG2D (Hinge (SEQ ID NO: 34)/TM (SEQ ID NO: 35))
TCCACAAGAATCAAGATCTTCCCTCTCTGAGCAGGAATCCTTTGTGCAT
TGAAGACTTTAGATTCCTCTCTGCGGTAGACGTGCACTTATAAGTATTT
GATGGGGTGGATTCGTGGTCGGAGGTCTCGACACAGCTGGGAGATGAGT
GAATTTCATAATTATAACTTGGATCTGAAGAAGAGTGATTTTTCAACAC
GATGGCAAAAGCAAAGATGTCCAGTAGTCAAAAGCAAATGTAGAGAAAA
TGCATCT/CCATTTTTTTTCTGCTGCTTCATCGCTGTAGCCATGGGAAT
CCGTTTCATTATTATGGTAACAATATGGAGT
FcγRIIIa (Hinge (SEQ ID NO: 36)/
TM (SEQ ID NO: 37))
CACCTGAGGTGTCACAGCTGGAAGAACACTGCTCTGCATAAGGTCACAT
ATTTACAGAATGGCAAAGGCAGGAAGTATTTTCATCATAATTCTGACTT
CTACATTCCAAAAGCCACACTCAAAGACAGCGGCTCCTACTTCTGCAGG
GGGCTTTTTGGGAGTAAAAATGTGTCTTCAGAGACTGTGAACATCACCA
TCACTCAAGGTTTGGCAGTGTCAACCATCTCATCATTCTTTCCACCTGG
GTACCAA/GTCTCTTTCTGCTTGGTGATGGTACTCCTTTTTGCAGTGGA
CACAGGACTATATTTCTCTGTGAAGACAAACA
NKp44 (Hinge (SEQ ID NO: 38)/TM (SEQ ID NO: 39))
TGTAGAATCTACCGCCCTTCTGACAACTCTGTCTCTAAGTCCGTCAGAT
TCTATCTGGTGGTATCTCCAGCCTCTGCCTCCACACAGACCTCCTGGAC
TCCCCGCGACCTGGTCTCTTCACAGACCCAGACCCAGAGCTGTGTGCCT
CCCACTGCAGGAGCCAGACAAGCCCCTGAGTCTCCATCTACCATCCCTG
TCCCTTCACAGCCACAGAACTCCACGCTCCGCCCTGGCCCTGCAGCCCC
CATTGCC/CTGGTGCCTGTGTTCTGTGGACTCCTCGTAGCCAAGAGCCT
GGTGCTGTCAGCCCTGCTCGTCTGGTGGGGG
NKp30 (Hinge (SEQ ID NO: 40)/TM (SEQ ID NO: 41))
TCCGTCACGTGGTTCCGAGATGAGGTGGTTCCAGGGAAGGAGGTGAGGA
ATGGAACCCCAGAGTTCAGGGGCCGCCTGGCCCCACTTGCTTCTTCCCG
TTTCCTCCATGACCACCAGGCTGAGCTGCACATCCGGGACGTGCGAGGC
CATGACGCCAGCATCTACGTGTGCAGAGTGGAGGTGCTGGGCCTTGGTG
TCGGGACAGGGAATGGGACTCGGCTGGTGGTGGAGAAAGAACATCCTCA
GCTAGGG/GCTGGTACAGTCCTCCTCCTTCGGGCTGGATTCTATGCTGT
CAGCTTTCTCTCTGTGGCCGTGGGCAGCACC
NKp46 (Hinge (SEQ ID NO: 42)/TM (SEQ ID NO: 43))
TTCCCCCTGGGCCCTGTGACCACAGCCCACAGAGGGACATACCGATGTT
TTGGCTCCTATAACAACCATGCCTGGTCTTTCCCCAGTGAGCCAGTGAA
GCTCCTGGTCACAGGCGACATTGAGAACACCAGCCTTGCACCTGAAGAC
CCCACCTTTCCTGCAGACACTTGGGGCACCTACCTTTTAACCACAGAGA
CGGGACTCCAGAAAGACCATGCCCTCTGGGATCACACTGCCCAGAATCT
CCTTCGG/ATGGGCCTGGCCTTTCTAGTCCTGGTGGCTCTAGTGTGGTT
CCTGGTTGAAGACTGGCTCAGCAGGAAGAGG
Activating KIR (KIR2DS4) (Hinge (SEQ ID NO: 44)/
TM (SEQ ID NO: 45))
AGGGAAGGGGAGGCCCATGAACGTAGGCTCCCTGCAGTGCGCAGCATCA
ACGGAACATTCCAGGCCGACTTTCCTCTGGGCCCTGCCACCCACGGAGG
GACCTACAGATGCTTCGGCTCTTTCCGTGACGCTCCCTACGAGTGGTCA
AACTCGAGTGATCCACTGCTTGTTTCCGTCACAGGAAACCCTTCAAATA
GTTGGCCTTCACCCACTGAACCAAGCTCCAAAACCGGTAACCCCAGACA
CCTACAT/GTTCTGATTGGGACCTCAGTGGTCAAAATCCCTTTCACCAT
CCTCCTCTTCTTTCTCCTTCATCGCTGG
NKG2C (Hinge (SEQ ID NO: 46)/TM (SEQ ID NO: 47))
ATGAATAAACAAAGAGGAACCTTCTCAGAAGTGAGTCTGGCCCAGGACC
CAAAGCGGCAGCAAAGGAAACCTAAAGGCAATAAAAGCTCCATTTCAGG
AACCGAACAGGAAATATTCCAAGTAGAATTAAATCTTCAAAATCCTTCC
CTGAATCATCAAGGGATTGATAAAATATATGACTGCCAAGGTTTACTGC
CACCTCCAGAGAAG/CTCACTGCCGAGGTCCTAGGAATCATTTGCATTG
TCCTGATGGCCACTGTGTTAAAAACAATAGTTCTTATTCCTTTC
CD8a (Hinge (SEQ ID NO: 48)/TM (SEQ ID NO: 49))
GTCCTCACCCTGAGCGACTTCCGCCGAGAGAACGAGGGCTACTATTTCT
GCTCGGCCCTGAGCAACTCCATCATGTACTTCAGCCACTTCGTGCCGGT
CTTCCTGCCAGCGAAGCCCACCACGACGCCAGCGCCGCGACCACCAACA
CCGGCGCCCACCATCGCGTCGCAGCCCCTGTCCCTGCGCCCAGAGGCGT
GCCGGCCAGCGGCGGGGGGCGCAGTGCACACGAGGGGGCTGGACTTCGC
CTGTGAT/ATCTACATCTGGGCGCCCTTGGCCGGGACTTGTGGGGTCCT
TCTCCTGTCACTGGTTATCACCCTTTACTGC
IL15Rb (Hinge (SEQ ID NO: 50)/TM (SEQ ID NO: 51))
GCCTCCCACTACTTTGAAAGACACCTGGAGTTCGAGGCCCGGACGCTGT
CCCCAGGCCACACCTGGGAGGAGGCCCCCCTGCTGACTCTCAAGCAGAA
GCAGGAATGGATCTGCCTGGAGACGCTCACCCCAGACACCCAGTATGAG
TTTCAGGTGCGGGTCAAGCCTCTGCAAGGCGAGTTCACGACCTGGAGCC
CCTGGAGCCAGCCCCTGGCCTTCAGGACAAAGCCTGCAGCCCTTGGGAA
GGACACC/ATTCCGTGGCTCGGCCACCTCCTCGTGGGCCTCAGCGGGGC
TTTTGGCTTCATCATCTTAGTGTACTTGCTGATCAACTGCAGG
CD3E (TM (SEQ ID NO: 67))
GTGATGTCCGTGGCCACCATCGTGATCGTGGACATCTGCATCACCGGCG
GCCTGCTGCTGCTGGTGTACTACTGGTCC
CD3G (TM (SEQ ID NO: 68))
GGCTTCCTGTTCGCCGAGATCGTGTCCATCTTCGTGCTGGCCGTGGGCG
TGTACTTCATCGCC
CD3D (TM (SEQ ID NO: 69))
GGCATCATCGTGACCGACGTGATCGCCACCCTGCTGCTGGCCCTGGGCG
TGTTCTGCTTCGCC

In some embodiments, the hinge used in a CAR construct of the present disclosure is a CD8a hinge sequence set forth below, or a functional fragment or variant thereof.

CD8a Hinge
(SEQ ID NO: 33)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT

### Linkers

In some embodiments, a linker is a polypeptide of any suitable length that can be used to covalently link any of the extracellular, transmembrane, or intracellular domains to generate a single chain polypeptide. In some embodiments, the linker preserves the function of the domains that are linked. In some embodiments, the linker is a gly-ser linker. In some embodiments, the linker is a gly-ser linker that is 15 amino acid or 20 amino acid in length. In some embodiments, the linker has one of the two linker sequences set forth below.

15 amino acid GS linker
(SEQ ID NO: 52)
GGGGSGGGGSGGGGS
20 amino acid GS linker
(SEQ ID NO: 53)
GGGGSGGGGSGGGGSGGGGS

### Bicistronic Vectors and Bicistronic Receptors

In some embodiments, bicistronic vectors are vectors comprising a single transcript, wherein the transcript allows the simultaneous expression of two proteins separately. Given that cell surface expression of CD3e may be stabilized through association with the ECD of other CD3 subunits or other protein ECDs, expression of an additional chimeric receptor with an ECD with such a stabilizing effect on CD3e would be advantageous. The present disclosure describes such bicistronic receptors, wherein they comprise a first and a second single chain chimeric receptor and are encoded by a bicistronic vector. Therefore, they are expressed simultaneously, enabling the proper association of the ECDs of the two chimeric receptors and trafficking to the cell surface for stable expression of the CD3e-containing bicistronic receptor. In some embodiments, the first and second chimeric receptors of the bicistronic receptors are linked with a suitable linker of any length that preserves the function of all the extracellular, transmembrane, and intracellular domains. In some embodiments, the suitable linker is a cleavable linker. In some embodiments, the cleavable linker is P2A. In some embodiments, the linker has the linker sequence set forth below.

P2A cleavable linker
(SEQ ID NO: 54)
GSGATNFSLLKQAGDVEENPGP

In some embodiments, the present disclosure provides a bicistronic receptor construct comprising a first and a second chimeric receptor, wherein each receptor comprises at least one extracellular domain, at least one intracellular domain, and a transmembrane domain as specified in rows 1-7 of Table 3.

TABLE 3

| | | | Intracellular | | | Intracellular |
|---|---|---|---|---|---|---|
| Construct | Extracellular | Transmembrane | signaling | Extracellular | Transmembrane | signaling |
| # | domain 1 | domain 1 | domain 1 | domain 2 | domain 2 | domain 2 |
| 1 | CD3E | CD3E | CD3E | CD3G | CD3G | CD3G |
| 2 | CD3E | CD3E | CD79A_2B4 | CD3G | CD3G | CD79B |
| 3 | CD3E | CD3E | CD79A_ | CD3G | CD3G | CD79B_IL2RB |
| | | | CD132_2B4 | | | |
| 4 | CD3E | CD3E | CD3Z | CD3G | CD3G | 41BB |
| 5 | CD3E | CD3E | CD3Z_CD132 | CD3G | CD3G | 41BB_IL2RB |
| 6 | CD3E | CD3E | CD3E | CD3E | CD3E | CD3E |
| 7 | CD3E | CD3E | CD3E | CD3D | CD3D | CD3D |
| 8 | CD3E | CD3E | 2B4_CD3Z | CD3G | CD3G | OX40 |

Below is an illustrative polypeptide sequence for a bicistronic receptor (SEQ ID NO. 55). It will be understood that the sequence described below is nonlimiting and can be modified through insertion, deletion, or substitution of amino acids or other methods of modification which enhances or preserves activity and functionality of the receptor or any of its domains.

Bicistronic receptor_WU76E
(SEQ ID NO: 55)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSRVKESRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPRGSGATNFSLLKQAGDVEENPGPMEQGKGLA
VLILAIILLQGTLAQSIKGNHLVKVYDYQEDGSVLLTCDAEAKNITWFK
DGKMIGFLTEDKKKWNLGSNAKDPRGMYQCKGSQNKSKPLQVYYRMCQN
CIELNAATISGFLFAEIVSIFVLAVGVYFIAKRGRKKLLYIFKQPFMRP
VQTTQEEDGCSCRFPEEEEGGCEL

More illustrative polypeptide sequences for bicistronic receptors and components thereof are provided below. It will be understood that the sequences described below are nonlimiting and can be modified through insertion, deletion, or substitution of amino acids or other methods of modification which enhances or preserves activity and functionality of the receptor or any of its domains.

CD3 Epsilon (Signal peptide, extracellular
domain, and transmembrane)
(SEQ ID NO: 56)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWS
CD3 Zeta Intracellular domain
(SEQ ID NO: 57)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
CD3 Gamma (Signal peptide, extracellular domain,
and transmembrane)
(SEQ ID NO: 58)
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCONCIELNAATISGFLFAEIVSIFVLAVGVYFIA
41BB Intracellular domain
(SEQ ID NO: 59)
KRGRKKLLYIFKQPFMRPVQTTQEEDGCSCRFPEEEEGGCEL
CD3 Epsilon (Signal peptide, extracellular
domain, and transmembrane)/CD3 zeta intracellular
domain
(SEQ ID NO: 60)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWS/RVKFSRSADAPAYKQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPR
CD3 Epsilon (Signal peptide, extracellular
domain, and transmembrane)/CD3 zeta intracellular
domain/P2A Linker
(SEQ ID NO: 61)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWS/RVKFSRSADAPAYKQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPR/GSGATNFSLLKQAGDVEENPGP
CD3 Epsilon (Signal peptide, extracellular
domain, and transmembrane)/CD3 zeta intracellular
domain/P2A Linker/CD3 Gamma (Signal peptide,
extracellular domain, and transmembrane)
(SEQ ID NO: 62)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWS/RVKFSRSADAPAYKQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPR/GSGATNFSLLKQAGDVEENPGP/MEQGK
GLAVLILAIILLQGTLAQSIKGNHLVKVYDYQEDGSVLLTCDAEAKNIT
WFKDGKMIGFLTEDKKKWNLGSNAKDPRGMYQCKGSQNKSKPLQVYYRM
CQNCIELNAATISGFLFAEIVSIFVLAVGVYFIA
CD3 Gamma (Signal peptide, extracellular domain,
and transmembrane)/41BB Intracellular domain
(SEQ ID NO: 63)
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIA/KRGRKKLLY
IFKQPFMRPVQTTQEEDGCSCRFPEEEEGGCEL
Bicistronic receptor WU76C
CD3E_CD79A_2B4_P2A_CD3G_CD79B
(SEQ ID NO. 70)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSRKRWQNEKLGLDAGDEYEDENLYEGLNLDQTATYEDISRGLQGT
YQDVGSLNIGDVQLEKPWRRKRKEKQSETSPKEFLTIYEDVKDLKTRRN
HEQEQTFPGGGSTIYSMIQSQSSAPTSQEPAYTLYSLIQPSRKSGSRKR
NHSPSFNSTIYEVIGKSQPKAQNPARLSRKELENFDVYSGSGATNFSLL
KQAGDVEENPGPMEQGKGLAVLILAIILLQGTLAQSIKGNHLVKVYDYQ
EDGSVLLTCDAEAKNITWFKDGKMIGFLTEDKKKWNLGSNAKDPRGMYQ
CKGSQNKSKPLQVYYRMCQNCIELNAATISGFLFAEIVSIFVLAVGVYF
IALDKDDSKAGMEEDHTYEGLDIDQTATYEDIVTLRTGEVKWSVGEHPG
QE
Bicistronic receptor WU76C
CD3E_CD79A_CD132_2B4_P2A_CD3G_CD79B_IL2RB
(SEQ ID NO. 71)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSRKRWQNEKLGLDAGDEYEDENLYEGLNLDQTATYEDISRGLQGT
YQDVGSLNIGDVQLEKPFWLERTMPRIPTLKNLEDLVTEYHGNFSAWSG
VSKGLAESLQPDYSERLCLVSEIPPKGGALGEGPGASPCNQHSPYWAPP
CYTLKPETWRRKRKEKQSETSPKEFLTIYEDVKDLKTRRNHEQEQTFPG
GGSTIYSMIQSQSSAPTSQEPAYTLYSLIQPSRKSGSRKRNHSPSFNST
IYEVIGKSQPKAQNPARLSRKELENFDVYSGSGATNFSLLKQAGDVEEN
PGPMEQGKGLAVLILAIILLQGTLAQSIKGNHLVKVYDYQEDGSVLLTC
DAEAKNITWFKDGKMIGFLTEDKKKWNLGSNAKDPRGMYQCKGSQNKSK
PLQVYYRMCQNCIELNAATISGFLFAEIVSIFVLAVGVYFIALDKDDSK
AGMEEDHTYEGLDIDQTATYEDIVTLRTGEVKWSVGEHPGQENCRNTGP
WLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLP
DALEIEACQVYFTYDPYSEEDPDEGVAGAPTGSSPQPLQPLSGEDDAYC
TFPSRDDLLLESPSLLGGPSPPSTAPGGSGAGEERMPPSLQERVPRDWD
PQPLGPPTPGVPDLVDFQPPPELVLREAGEEVPDAGPREGVSFPWSRPP
GQGEFRALNARLPLNTDAYLSLQELQGQDPTHLV
Bicistronic receptor WU76E CD3E_3Z_P2A_CD3G_41BB
CAR
(SEQ ID NO. 55)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSRVKFSRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPRGSGATNFSLLKQAGDVEENPGPMEQGKGLA
VLILAIILLQGTLAQSIKGNHLVKVYDYQEDGSVLLTCDAEAKNITWFK
DGKMIGFLTEDKKKWNLGSNAKDPRGMYQCKGSQNKSKPLQVYYRMCQN
CIELNAATISGFLFAEIVSIFVLAVGVYFIAKRGRKKLLYIFKQPFMRP
VQTTQEEDGCSCRFPEEEEGGCEL.
Bicistronic receptor WU76F
CD3E_3Z_CD132_P2A_CD3G_41BB_IL2RB
(SEQ ID NO. 72)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSRVKFSRSADAPAYKQGQNQLYNELNLGRREEYDVLDKRRGRDPE
MGGKPRRKNPQEGLYNELQKDKMAEAYSEIGMKGERRRGKGHDGLYQGL
STATKDTYDALHMQALPPRFWLERTMPRIPTLKNLEDLVTEYHGNFSAW
SGVSKGLAESLQPDYSERLCLVSEIPPKGGALGEGPGASPCNQHSPYWA
PPCYTLKPETGSGATNFSLLKQAGDVEENPGPMEQGKGLAVLILAIILL
QGTLAQSIKGNHLVKVYDYQEDGSVLLTCDAEAKNITWFKDGKMIGFLT
EDKKKWNLGSNAKDPRGMYQCKGSQNKSKPLQVYYRMCQNCIELNAATI
SGFLFAEIVSIFVLAVGVYFIAKRGRKKLLYIFKQPFMRPVQTTQEEDG
CSCRFPEEEEGGCELNCRNTGPWLKKVLKCNTPDPSKFFSQLSSEHGGD
VQKWLSSPFPSSSFSPGGLAPEISPLEVLERDKVTQLLLQQDKVPEPAS
LSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPG
GSGAGEERMPPSLQERVPRDWDPQPLGPPTPGVPDLVDFQPPPELVLRE
AGEEVPDAGPREGVSFPWSRPPGQGEFRALNARLPLNTDAYLSLQELQG
QDPTHLV
Bicistronic receptor WU76UB
CD3E_CD3E_2B4_CD3Z_CD3G_CD3G_OX40
(SEQ ID NO: 74)
ATGCAGTCCGGCACCCACTGGCGCGTGCTGGGCCTGTGCCTGCTGTCCG
TGGGCGTGTGGGGCCAGGACGGCAACGAGGAGATGGGCGGCATCACCCA
GACCCCCTACAAGGTGTCCATCTCCGGCACCACCGTGATCCTGACCTGC
CCCCAGTACCCCGGCTCCGAGATCCTGTGGCAGCACAACGACAAGAACA
TCGGCGGCGACGAGGACGACAAGAACATCGGCTCCGACGAGGACCACCT
GTCCCTGAAGGAGTTCTCCGAGCTGGAGCAGTCCGGCTACTACGTGTGC
TACCCCCGCGGCTCCAAGCCCGAGGACGCCAACTTCTACCTGTACCTGC
GCGCCCGCGTGTGCGAGAACTGCATGGAGATGGACGTGATGTCCGTGGC
CACCATCGTGATCGTGGACATCTGCATCACCGGCGGCCTGCTGCTGCTG
GTGTACTACTGGTCCGGCAGTGGCtggcgccgcaagcgcaaggagaagc
agtccgagacctcccccaaggagttcctgaccatctacgaggacgtgaa
ggacctgaagacccgccgcaaccacgagcaggagcagaccttccccggg
gggctccaccatctactccatgatccagtcccagtcctccgcccccacc
tcccaggagcccgcctacaccctgtactccctgatccagccctcccgca
agtccggctcccgcaagcgcaaccactccccctccttcaactccaccat
ctacgaggtgatcggcaagtcccagcccaaggcccagaaccccgcccgc
ctgtcccgcaaggagctggagaacttcgacgtgtactccGGCAGTGGCC
GCGTGAAGTTCTCCCGCTCCGCCGACGCCCCCGCCTACAAGCAGGGCCA
GAACCAGCTGTACAACGAGCTGAACCTGGGCCGCCGCGAGGAGTACGAC
GTGCTGGACAAGCGCCGCGGCCGCGACCCCGAGATGGGCGGCAAGCCCC
GCCGCAAGAACCCCCAGGAGGGCCTGTACAACGAGCTGCAGAAGGACAA
GATGGCCGAGGCCTACTCCGAGATCGGCATGAAGGGCGAGCGCCGCCGC
GGCAAGGGCCACGACGGCCTGTACCAGGGCCTGTCCACCGCCACCAAGG
ACACCTACGACGCCCTGCACATGCAGGCCCTGCCCCCCCGCggaagcgg
agccacgaacttctctctgttaaagcaagcaggagatgttgaagaaaac
cccgggcctATGGAGCAGGGCAAGGGCCTGGCCGTGCTGATCCTGGCCA
TCATCCTGCTGCAGGGCACCCTGGCCCAGTCCATCAAGGGCAACCACCT
GGTGAAGGTGTACGACTACCAGGAGGACGGCTCCGTGCTGCTGACCTGC
GACGCCGAGGCCAAGAACATCACCTGGTTCAAGGACGGCAAGATGATCG
GCTTCCTGACCGAGGACAAGAAGAAGTGGAACCTGGGCTCCAACGCCAA
GGACCCCCGCGGCATGTACCAGTGCAAGGGCTCCCAGAACAAGTCCAAG
CCCCTGCAGGTGTACTACCGCATGTGCCAGAACTGCATCGAGCTGAACG
CCGCCACCATCTCCGGCTTCCTGTTCGCCGAGATCGTGTCCATCTTCGT
GCTGGCCGTGGGCGTGTACTTCATCGCCGGCTCCGGCGCCCTGTACCTG
CTGCGCCGCGACCAGCGCCTGCCCCCCGACGCCCACAAGCCCCCCGGCG
GCGGCTCCTTCCGCACCCCCATCCAGGAGGAGCAGGCCGACGCCCACTC
CACCCTGGCCAAGATCTAA

### Tricistronic Vectors and Tricistronic Receptors

In some embodiments, tricistronic vectors are vectors comprising a single transcript, where the transcript allows the simultaneous expression of three proteins separately. Given that cell surface expression of CD3e may be stabilized through association with the ECD of other CD3 subunits or other protein ECDs, expression of a second and a third chimeric receptor with an ECD with such a stabilizing effect on the CD3e ECD of the first chimeric receptor would be advantageous. Moreover, additional chimeric receptors can provide additional potential payload area for membrane-proximal intracellular signaling domains. The present disclosure describes such tricistronic receptors that comprise a first, a second, and a third single chain chimeric receptor and are encoded by a tricistronic vector. These chains may be expressed simultaneously, allowing the proper association of the ECDs of the three single chain chimeric receptors and trafficking to the cell surface for stable expression of the CD3e-containing tricistronic receptor. In some embodiments, the first, the second, and/or the third chimeric receptors of the tricistronic receptors are linked with a suitable linker of any length that preserves the function of all the extracellular, transmembrane, and intracellular domains. In some embodiments, the suitable linker is a cleavable linker. In some embodiments, the cleavable linker is P2A. In some embodiments, the linker has the linker sequence set forth below.

P2A cleavable linker
(SEQ ID NO: 54)
GSGATNFSLLKQAGDVEENPGP

Below is an illustrative polypeptide sequence for a tricistronic receptor (SEQ ID NO. 75). It will be understood that the sequence described below is nonlimiting and can be modified through insertion, deletion, or substitution of amino acids or other methods of modification which enhances or preserves activity and functionality of the receptor or any of its domains.

Tricistronic receptor WU76Z
(SEQ ID NO: 75)
ATGCAGTCCGGCACCCACTGGCGCGTGCTGGGCCTGTGCCTGCTGTCCG
TGGGCGTGTGGGGCCAGGACGGCAACGAGGAGATGGGCGGCATCACCCA
GACCCCCTACAAGGTGTCCATCTCCGGCACCACCGTGATCCTGACCTGC
CCCCAGTACCCCGGCTCCGAGATCCTGTGGCAGCACAACGACAAGAACA
TCGGCGGCGACGAGGACGACAAGAACATCGGCTCCGACGAGGACCACCT
GTCCCTGAAGGAGTTCTCCGAGCTGGAGCAGTCCGGCTACTACGTGTGC
TACCCCCGCGGCTCCAAGCCCGAGGACGCCAACTTCTACCTGTACCTGC
GCGCCCGCGTGTGCGAGAACTGCATGGAGATGGACGTGATGTCCGTGGC
CACCATCGTGATCGTGGACATCTGCATCACCGGCGGCCTGCTGCTGCTG
GTGTACTACTGGTCCCGCGTGAAGTTCTCCCGCTCCGCCGACGCCCCCG
CCTACAAGCAGGGCCAGAACCAGCTGTACAACGAGCTGAACCTGGGCCG
CCGCGAGGAGTACGACGTGCTGGACAAGCGCCGCGGCCGCGACCCCGAG
ATGGGCGGCAAGCCCCGCCGCAAGAACCCCCAGGAGGGCCTGTACAACG
AGCTGCAGAAGGACAAGATGGCCGAGGCCTACTCCGAGATCGGCATGAA
GGGCGAGCGCCGCCGCGGCAAGGGCCACGACGGCCTGTACCAGGGCCTG
TCCACCGCCACCAAGGACACCTACGACGCCCTGCACATGCAGGCCCTGC
CCCCCCGCggaagcggagccacgaacttctctctgttaaagcaagcagg
agatgttgaagaaaaccccgggcctATGGAGCAGGGCAAGGGCCTGGCC
GTGCTGATCCTGGCCATCATCCTGCTGCAGGGCACCCTGGCCCAGTCCA
TCAAGGGCAACCACCTGGTGAAGGTGTACGACTACCAGGAGGACGGCTC
CGTGCTGCTGACCTGCGACGCCGAGGCCAAGAACATCACCTGGTTCAAG
GACGGCAAGATGATCGGCTTCCTGACCGAGGACAAGAAGAAGTGGAACC
TGGGCTCCAACGCCAAGGACCCCCGCGGCATGTACCAGTGCAAGGGCTC
CCAGAACAAGTCCAAGCCCCTGCAGGTGTACTACCGCATGTGCCAGAAC
TGCATCGAGCTGAACGCCGCCACCATCTCCGGCTTCCTGTTCGCCGAGA
TCGTGTCCATCTTCGTGCTGGCCGTGGGCGTGTACTTCATCGCCAAGCG
CGGCCGCAAGAAGCTGCTGTACATCTTCAAGCAGCCCTTCATGCGCCCC
GTGCAGACCACCCAGGAGGAGGACGGCTGCTCCTGCCGCTTCCCCGAGG
AGGAGGAGGGCGGCTGCGAGCTGGGAAGCGGAGAGGGCAGGGGAAGTCT
TCTAACATGCGGGGACGTGGAGGAAAATCCCGGCCCCATGGAGCACTCC
ACCTTCCTGTCCGGCCTGGTGCTGGCCACCCTGCTGTCCCAGGTGTCCC
CCTTCAAGATCCCCATCGAGGAGCTGGAGGACCGCGTGTTCGTGAACTG
CAACACCTCCATCACCTGGGTGGAGGGCACCGTGGGCACCCTGCTGTCC
GACATCACCCGCCTGGACCTGGGCAAGCGCATCCTGGACCCCCGCGGCA
TCTACCGCTGCAACGGCACCGACATCTACAAGGACAAGGAGTCCACCGT
GCAGGTGCACTACCGCATGTGCCAGTCCTGCGTGGAGCTGGACCCCGCC
ACCGTGGCCGGCATCATCGTGACCGACGTGATCGCCACCCTGCTGCTGG
CCCTGGGCGTGTTCTGCTTCGCCtggcgccgcaagcgcaaggagaagca
gtccgagacctcccccaaggagttcctgaccatctacgaggacgtgaag
gacctgaagacccgccgcaaccacgagcaggagcagaccttccccgggg
ggctccaccatctactccatgatccagtcccagtcctccgcccccacct
cccaggagcccgcctacaccctgtactccctgatccagccctcccgcaa
gtccggctcccgcaagcgcaaccactccccctccttcaactccaccatc
tacgaggtgatcggcaagtcccagcccaaggcccagaaccccgcccgcc
tgtcccgcaaggagctggagaacttcgacgtgtactccTAA

### T Cell Engagers

A T cell engager is a molecule that acts as a bridge between a target cell and a T cell. A bispecific T cell engager (BiTE) is a T cell engager with bispecific affinity to two antigens/ligands. A wide variety of molecules have been developed which are based on the basic concept of having two antibody-like binding domains. BiTEs are a class of bispecific antibody-type molecules that have been developed, primarily for the use as anti-cancer drugs. They direct a host's immune system, such as the T cells' cytotoxic activity, against a target cell, such as a cancer cell. In these molecules, one binding domain binds to a T cell via the CD3 receptor, and the other binding domain binds to a target cell such as a tumor cell (via targeting of a tumor specific antigen). Since the bispecific molecule binds both the target cell and the T cell, it brings the target cell into proximity with the T cell, so that the T cell can exert its effect, for example, a cytotoxic effect on a cancer cell. The formation of the T cell:bispecific Ab:cancer cell complex induces signaling in the T cell leading to, for example, the release of cytotoxic mediators. Ideally, the agent only induces the desired signaling in the presence of the target cell, leading to selective killing. A BiTE is generally a fusion protein comprising two single-chain variable fragments (scFvs) of different antibodies. In some embodiments, a BiTE comprises a spacer sequence to connect the first domain with the second domain and spatially separate the two domains. The terms “bispecific T cell engager” or “BiTE” or “BiTEs” in the present disclosure refers to a molecule with two antigen binding domains, which can bind the same or different antigens. In some embodiments, the BiTE comprises an anti-CD3 binding domain and a further binding domain or the BiTE comprises an anti-CD28 binding domain and a further binding domain. Other T cell engagers include, but are not limited to, a trispecific T cell engager (TriTE), a dual affinity retargeting antibody (DART), a TeTriTE, and a quadrispecific T cell engagers. In some embodiments, the antigen binding regions of these engagers can bind the same or different antigens. In some embodiments, the T cell engager binding the extracellular domain comprises at least one antigen binding region that binds CD3 or CD28.

### Bispecific Natural Killer Cell Engagers

A natural killer cell engager (NKE) is a molecule that acts as a bridge between a target cell and a NK cell. Structurally, bispecific NKEs have affinity to two antigens/ligands, where one binding domain binds to a NK cell via an NK cell activating receptor such as CD16, NKG2D, NKp30, or NKp46, and the other binding domain binds to a target cell such as a tumor cell (via targeting of a tumor specific antigen). In some embodiments, the natural killer cell engager is a bispecific killer cell engager (BiKE). In some embodiments, the NKE is a trispecific killer cell engager (TriKE) or a TeTriKE.

### Killer Cells

In some embodiments, the constructs of the present disclosure are engineered for enhanced activity and performance in NK cells and T cells.

The term “T cells” can refer generally to T cells and subtypes thereof, such as naïve T cells, CD4+ T cells, helper T cells, CD8+ T cells, cytotoxic or killer T cells, Th1, Th2, Th9, Th17, central memory, and effector memory T cells and variations thereof, any of which may be derived from various sources, including peripheral or cord blood cells, stem cells, induced pluripotent stem cells (iPSCs), and immortalized T cells such as Jurkat cells. In some embodiment, the immune cells of the present disclosure have been engineered or genetically modified to mitigate allogeneic rejection by a recipient into whom the cells are introduced. In some embodiments, the immune cells of the present disclosure are engineered to be deficient in expression, activity, or signaling, relative to corresponding cells that have not been so engineered, of molecules that mediate allogeneic rejection.

In some embodiments, the T cell is genetically modified to be deficient in an endogenous gene. In some embodiments, the endogenous TCR is deleted from the T cell through gene editing. In some embodiments, the TCR is deleted through TRAC gene editing. In some embodiments, the TCR is modified, deleted, or silenced by molecular biology methods. In some embodiments, the TCR is deleted by CRISPR/Cas9 mediated gene knockout.

The term “NK cells” can refer generally to NK cells and subtypes thereof, such as memory NK cells, memory-like (ML) NK cells, and cytokine-induced memory-like (CIML) NK cells, and variations thereof, any of which may be derived from various sources, including peripheral or cord blood cells, stem cells, induced pluripotent stem cells (iPSCs), and immortalized NK cells such as NK-92 cells. In some embodiments, the engineered NK cells of the present disclosure are deficient in NKG2A and/or CD8 expression, activity, or signaling.

### NK Cells

NK cells are generally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. NK cells are an alternative to T cells for allogeneic cellular immunotherapy since, in some instances, they have been administered safely without severe toxicity. In general, NK cells do not cause graft versus host disease (GvHD), can naturally recognize and remove malignant cells, and are amendable to cellular engineering.

### Memory, Memory-Like, and CIML NK Cells

In addition to their innate cytotoxic and immunostimulatory activity, NK cells constitute a heterogeneous and versatile cell subset, including persistent memory NK populations, in some cases also called memory-like or cytokine-induced-memory-like (CIML) NK cells, that mount robust recall responses. Memory NK cells can be produced by stimulation by pro-inflammatory cytokines or activating receptor pathways, either naturally or artificially (“priming”). Memory NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated memory NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+NK cells were reduced. Finally, unlike in vitro differentiated ML NK cells, in vivo differentiated ML NK cells did not express CD25.

### Cytokine-Induced Memory-Like Natural Killer Cells (CIML-NKs)

In some embodiments, NK cells are induced to acquire a memory-like phenotype, for example, by priming (preactivation) with combinations of cytokines, such as interleukin-12 (IL-12), IL-15, and IL-18. These cytokine-induced memory-like NK cells (CIML-NK) exhibit enhanced response upon restimulation with the cytokines or engagement of activating receptors. In some embodiments, CIML-NK cells are produced by activation with cytokines such as IL-12, IL-15, and IL-18 and/or their related family members, or functional fragments thereof, or fusion proteins comprising functional fragments thereof.

Memory NK cells typically exhibit differential cell surface protein expression patterns when compared to conventional NK cells. Such expression patterns are known in the art and can comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML-NK cells (see e.g., Romee et al. Sci Transl Med. 2016 Sep. 21; 8(357):357). In some embodiments, memory NK cells are identified by observed in vitro and in vivo properties, such as enhanced effector functions such as cytotoxicity, improved persistence and increased IFN-γ production when compared to a heterogenous NK cell population.

The NK cells used according to the present disclosure can be prepared using any known methodologies. For example, in some embodiments, the isolated NK cells can be activated using cytokines, such as IL-12/15/18. The NK cells can be incubated in the presence of the cytokines for an amount of time sufficient to form CIML-NK cells. For example, the amount of time sufficient to form CIML-NK cells can be between about 8 and about 24 hours, about 12 hours, or about 16 hours. As another example, the amount of time sufficient to form cytokine-activated memory-like (ML) NK cells can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

In some embodiments, the chimeric antigen receptor (CAR) can then be transduced via a viral vector (e.g., lentivirus) into the CIML-NK cells in the presence of IL-15 for an amount of time sufficient to virally transduce CAR into the CIML-NK cells, resulting in CAR-transduced ML NK cells. For example, the amount of time sufficient to form CAR-transduced ML NK cells can be between about 12 hours and about 24 hours. As another example, the amount of time sufficient to virally transduce CAR into the ML NK cells (forming CAR-transduced ML NK cells) can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

In some embodiments, the CAR-transduced ML NK cells can then be incubated in the presence of IL-15 for an amount of time sufficient to express the vector and to form CAR-expressing ML NK (CARML NK cells). For example, the amount of time sufficient to form CARML NK cells can be between about 3 days and about 8 days. As an example, the amount of time sufficient to form CARML NK cells can be at least about 1 day; about 2 days; about 3 days; about 4 days; about 5 days; about 6 days; about 7 days; about 8 days; about 9 days; about 10 days; about 11 days; about 12 days; about 13 days; or about 14 days.

In some embodiments, methods for preparing ML NK cells to be used according to the present disclosure include those described in WO2020/047299 and WO2020/047473, the contents of which are incorporated herein by reference in their entireties.

### Formulation

The agents and compositions described herein can be formulated by any suitable manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. In some embodiments, such formulations contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, epidural, ophthalmic, transdermal, buccal, and rectal. In some embodiments, the individual agents are administered in combination with one or more additional agents or together with other biologically active or biologically inert agents. Such biologically active or inert agents can be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating a proliferative disease, disorder, or condition, infectious disease, or immune disorder in a subject in need administration of a therapeutically effective amount of NK cell-based therapy (e.g., using genetically modified NK cells). The disclosed NK-cell based therapy can be used as a treatment for cancer (e.g., as an immunotherapy drug), for an autoimmune disease (e.g., treatment to deplete B cells), or for an infectious disease.

The scFvs described herein and the engineered NK cells described herein in combination with any bispecific target antigen-binding agent can be used for targeting cancer antigens associated with hematological malignancies such as AML, ALL, or Lymphoma, but can also be expanded for use in any malignancy, autoimmune, or infectious disease where a scFv or bispecific target antigen-binding agent can be generated against a target. For example, the constructs described herein can be used to treat or prevent autoimmunity associated with auto-antibodies (similar indications as rituximab for autoimmunity). As another example, the disclosed constructs can also be applied to virally infected cells, using a scFv or bispecific target antigen-binding agent that can recognize viral antigens, for example gp120 and gp41 on HIV-infected cells.

Methods described herein are generally performed on a subject in need thereof. A subject in need of the therapeutic methods described herein can be a subject having, diagnosed with, suspected of having, or at risk for developing a proliferative disease, disorder, or condition; an immune disorder; or an infectious disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a NK cell-based treatment is, for example, that amount that would cause the desired therapeutic effect in a subject while minimizing undesired side effects. In various embodiments, an effective amount of a NK cell-based treatment described herein can substantially inhibit a disease, disorder, or condition, slow the progress of a disease, disorder, or condition, or limit the development of a disease, disorder, or condition.

“Substantially” can be any large portion up to totality. Thus “substantially blocked or inhibited”, or “substantially removed” can be nearly or nearly completely blocked, inhibited, or removed.

According to the methods described herein, administration can be parenteral, pulmonary, oral, topical, intradermal, intramuscular, intraperitoneal, intravenous, subcutaneous, intranasal, epidural, ophthalmic, buccal, or rectal administration. Preferably, NK cells can be administered as an intravenous infusion.

When used in the treatments described herein, a therapeutically effective amount of a NK cell-based treatment can be employed in a purified form or, where such forms exist, in pharmaceutically acceptable form and with or without a pharmaceutically acceptable excipient. For example, the compounds of the present disclosure can be administered, at a reasonable benefit/risk ratio applicable to any medical treatment, in a sufficient amount to inhibit a disease, disorder, or condition, slow the progress of a disease, disorder, or condition, or limit the development of a disease, disorder, or condition.

The amount of NK cell-based treatment (e.g., CARML NK cells) described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by suitable pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be preferred.

The specific therapeutically effective dose level for any particular subject depends upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the NK cell-based treatment can occur as a single event or over a time course of treatment. For example, NK cell-based treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease, disorder, or condition, such as chemotherapy, immunotherapy, or checkpoint blockade therapy. For example, a subject can be administered at least one therapeutic agent selected from an interferon; a checkpoint inhibitor antibody; an antibody-drug conjugate (ADC); an anti-HLA-DR antibody; or an anti-CD74 antibody. Other examples can include a therapeutic agent selected from a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), a boron compound, or a radioisotope.

A NK cell-based treatment can be administered simultaneously or sequentially with another agent, such as an antibiotic, an anti-inflammatory, or another agent. For example, a NK cell-based treatment can be administered simultaneously with another agent, such as an antibiotic or an anti-inflammatory. Simultaneous administration can occur through administration of separate compositions, each containing one or more of a NK cell-based treatment, an antibiotic, an anti-inflammatory, or another agent. Simultaneous administration can occur through administration of one composition containing two or more of a NK cell-based treatment, an antibiotic, an anti-inflammatory, or another agent. A NK cell-based treatment can be administered sequentially with an antibiotic, an anti-inflammatory, or another agent. For example, a NK cell-based treatment can be administered before or after administration of an antibiotic, an anti-inflammatory, or another agent.

Methods and compositions as described herein can be used for the prevention, treatment, or slowing the progression of cancer, autoimmune conditions associated with autoantibodies, immune disorder, or infectious diseases (e.g., bacterial, viral). The disclosed CARML NK cell constructs can be designed to incorporate a targeting antibody fragment against a disease-associated antigen, such as scFvs that target cancer or an infectious disease. As described herein, targeting antibody fragments against a disease-associated antigens are well known.

For example, the cancer can a hematological cancer or a cancer with a solid tumor. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

As another example, the autoimmune condition or immune disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Bald disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; or Vogt-Koyanagi-Harada Disease.

As another example the autoimmune condition or immune disorder can be an autoinflammatory disease. The autoinflammatory can be Familial Mediterranean Fever (FMF), neonatal Onset Multisystem Inflammatory Disease (NOMID), Tumor Necrosis Factor Receptor-Associated Periodic Syndrome (TRAPS), Deficiency of the Interleukin-1 Receptor Antagonist (DIRA), Behçet's Disease, or Chronic Atypical Neutrophilic Dermatosis with Lipodystrophy and Elevated Temperature (CANDLE).

As another example, the treatment of an infectious disease can be any bacterial infection or viral infection, using a scFv that can recognize antigens, such as antigens on HIV infected cells. The infectious disease can be Acute Flaccid Myelitis (AFM); Anaplasmosis; Anthrax; Babesiosis; Botulism; Brucellosis; Campylobacteriosis; Carbapenem-resistant Infection (CRE/CRPA); Chancroid; Chikungunya Virus Infection (Chikungunya); Chlamydia; Ciguatera (Harmful Algae Blooms (HABs)); Clostridium Difficile Infection; Clostridium Perfringens (Epsilon Toxin); Coccidioidomycosis fungal infection (Valley fever); Creutzfeldt-Jacob Disease, transmissible spongiform encephalopathy (CJD); Cryptosporidiosis (Crypto); Cyclosporiasis; Dengue, 1,2,3,4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin—B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Tuberculosis (TB); Tuberculosis (Latent) (LTBI); Tularemia (Rabbit fever); Typhoid Fever, Group D; Typhus; Vaginosis, bacterial (Yeast Infection); Vaping-Associated Lung Injury (e-Cigarette Associated Lung Injury); Varicella (Chickenpox); Vibrio cholerae (Cholera); Vibriosis (Vibrio); Viral Hemorrhagic Fever (Ebola, Lassa, Marburg); West Nile Virus; Yellow Fever; Yersenia (Yersinia); or Zika Virus Infection (Zika).

### Administration

An aspect of the present disclosure provides for NK cells (e.g., CARML NK cells, modified NK cells, pre-activated NK cells, NKG2A-blocked NK cells, pre-activated and NKG2A-blocked NK cells) to be directly administered to a subject.

Apheresis (e.g., the removal of blood plasma from the body by the withdrawal of blood, its separation into plasma and cells, and the reintroduction of the cells) can be performed on the subject.

In some embodiments, the NK cells can be purified and activated with IL-12/IL-15/IL-18 for about 12 hours. The NK cells can be washed and transduced with CAR lentivirus (e.g., twice over about two days). The cells can be washed and infused into the patient at about 10⁷ cell/kg. In the haplo/allo setting the cells can be supported with rhIL-2 and in the autologous setting the cells can be supported with IL-15.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 mm), nanospheres (e.g., less than 1 mm), microspheres (e.g., 1-100 mm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems include, for example, an infusion pump which can be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

A “promoter” is generally understood as a nucleic acid control sequence that directs transcription of a nucleic acid. An inducible promoter is generally understood as a promoter that mediates transcription of an operably linked gene in response to a particular stimulus. A promoter can include nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs can also be constructed to be capable of expressing antisense RNA molecules, in order to inhibit translation of a specific RNA molecule of interest. For the practice of the present disclosure, suitable compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by and/or supports the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. In some embodiments, the two nucleic acid molecules are part of a single contiguous nucleic acid molecule. In some embodiments, the two nucleic acid molecules are adjacent. For example, a promoter is operably linked to a gene of interest if the promoter regulates or mediates transcription of the gene of interest in a cell. In another example, the domains present in a CAR construct according to the present disclosure can be said to be operably linked to one another so long as they carry out their intended function within the CAR.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked to another.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “vector” refers to a carrier for a nucleic acid, which can be used to introduce the nucleic acid into a cell. An “expression vector” is a vector that comprises a sequence encoding a protein or an RNA (e.g., a circular RNA) and any regulatory regions needed for expression of the sequence in a cell. In some embodiments, the sequence encoding a protein or an RNA is operably linked to another sequence in the vector. The term “operably linked” refers to the regulatory sequences necessary for expression of the sequence encoding a protein or an RNA are placed in the nucleic acid molecule in the appropriate positions relative to the sequence to effect expression of the protein or RNA.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

In some embodiments, directed evolution and rapid isolation of mutants are performed according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the desired activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Host cells can be transformed using a variety of techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

TABLE 2
Substitutions

| | |
|---|---|
| Side Chain Characteristic | Amino Acid |

Conservative Substitutions I

| | |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that can be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene is already present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The term “modified” refers to a substance or compound (e.g., a cell, a polynucleotide sequence, and/or a polypeptide sequence) that has been altered or changed as compared to the corresponding unmodified substance or compound.

The term “genetically modified” or “engineered” refers to a method of modifying the genome of a cell, including, but not limited to, deleting a coding or non-coding region or a portion thereof or inserting a coding region or a portion thereof. In some embodiments, the cell that is modified is a NK cell which can either be obtained from a patient or a donor. The cell can be modified to express an exogenous construct, such as a chimeric antigen receptor (CAR), which is incorporated into the cell's genome.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “polynucleotide” and “nucleic acid,” used interchangeably herein, refer to a polymeric form of nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of about 5 to about 100 nucleotides of single- or double-stranded DNA, However, in this disclosure, there is no upper or lower limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or anti sense) and double-stranded polynucleotides.

The term “conditioning” indicates preparing a patient in need of a NK or T cell therapy for a suitable condition. Methods of conditioning can include, but are not limited to, reducing the number of endogenous lymphocytes, removing a cytokine sink, increasing a serum level of one or more homeostatic cytokines or pro-inflammatory factors, enhancing an effector function of NK or T cells administered after the conditioning, enhancing antigen presenting cell activation and/or availability, or any combination thereof prior to a NK or T cell therapy.

The term “subject” includes animals, such as mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments, subjects are rodents (e.g., mice, rats, hamsters), rabbits, primates, or swine such as inbred pigs and the like. The terms “subject” and “patient” are used interchangeably herein.

The terms “treatment” or “treating” used to refer to treatment or treating of a subject indicate any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In some embodiments, “treatment” or “treating” includes a complete remission.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application, including US2020216859A1, US2017066827A1, and U.S. Pat. No. 9,855,298B2, are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims.

### Other Embodiments of the Disclosure

Notwithstanding the appended claims, the following numbered clauses also form part of the instant disclosure.

1. An engineered natural killer cell (NK cell) comprising a chimeric receptor comprising a CD3 epsilon (CD3e) extracellular domain, a first transmembrane domain, and a first intracellular domain; wherein the first transmembrane domain does not comprise a CD3e transmembrane domain.

2. An engineered natural killer cell (NK cell) comprising a chimeric receptor comprising a CD3 epsilon (CD3e) extracellular domain, a first transmembrane domain, and a first intracellular domain; wherein the first intracellular domain does not comprise a CD3e intracellular domain.

3. The engineered NK cell of clause 1 or clause 2, wherein the CD3e extracellular domain is noncovalently associated with or linked to a CD3 gamma (CD3g) extracellular domain or a CD3 delta (CD3d) extracellular domain by a linker.

4. The engineered NK cell of clause 3, wherein the linker comprises a polypeptide chain comprising a Gly Ser linker.

5. The engineered NK cell of clause 4, wherein the linker comprises the polypeptide sequence set forth in SEQ ID NO. 52 or SEQ ID NO. 53.

6. The engineered NK cell of any one of clause 1 to clause 5, wherein the CD3e extracellular domain is capable of binding to a T cell engager.

7. The engineered NK cell of clause 6, wherein the T cell engager is selected from the list consisting of a bispecific T cell engager (BiTE), a trispecific T cell engager (TriTE), a TeTriTE, and a dual affinity retargeting antibody (DART).

8. The engineered NK cell of any one of clause 6 to clause 7, wherein the T cell engager is capable of binding to a target selected from PSMA, MAGE-A4, HER2, GPC3, GD2, FLT3, EPCAM, DLL3, CLDN 18.2, CD38, CD33, CD20, CD19, CD123, BCMA, B&-H7, and Mesothelin.

9. The engineered NK cell of any one of clause 6 to clause 8, wherein the CD3e extracellular domain is capable of binding to a bispecific T cell engager (BiTE).

10. The engineered NK cell of clause 6, wherein the BiTE is Blinatumomab, Tebentafusp, Mosunetuzumab, Teclistamab, Cibisatamab, or Tarlatamab.

11. The engineered NK cell of clause 10, wherein the BiTE is Blinatumomab.

12. The engineered NK cell of any one of clause 1 to clause 11, wherein the CD3e extracellular domain comprises a polypeptide encoded by a nucleic acid comprising SEQ ID NO. 23, or a functional fragment or variant thereof having at least 90% identity to SEQ ID 23.

13. The engineered NK cell of any one of clause 3 to clause 12, wherein the CD3g extracellular domain or the CD3d extracellular domain is linked to a second transmembrane domain, wherein the second transmembrane domain is linked to a second intracellular domain.

14. The engineered NK cell of any one of clause 1 to clause 13, wherein the first transmembrane domain is selected from the group consisting of: CD3d, CD3e, CD3g, CD16, NKG2D, DAP10, FcγRIIIa, NKp44, NKp30, NKp46, actKIR, NKG2C, CD8a, and IL15 transmembrane domains.

15. The engineered NK cell of clause 14, wherein the first transmembrane domain comprises a CD3e transmembrane domain.

16. The engineered NK cell of any one of clause 13 to clause 15, wherein the second transmembrane domain is selected from the group consisting of: CD3d, CD3e, CD3g, CD16, NKG2D, DAP10, FcγRIIIa, NKp44, NKp30, NKp46, actKIR, NKG2C, CD8a, and IL15 transmembrane domains.

17. The engineered NK cell of clause 16, wherein the second transmembrane domain comprises a CD3g transmembrane domain.

18. The engineered NK cell of any one of clause 1 to clause 17, wherein the first intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

19. The engineered NK cell of clause 18, wherein the first intracellular signaling domain comprises a CD3z ITAM intracellular signaling domain.

20. The engineered NK cell of any one of clause 13 to clause 19, wherein the second intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

21. The engineered NK cell of clause 20, wherein the second intracellular signaling domain comprises a 4-1BB intracellular signaling domain.

22. The engineered NK cell of any one of clause 18 to clause 21, wherein the 4-1BB intracellular signaling domain comprises a polypeptide encoded by a nucleic acid comprising SEQ ID NO. 7, or a functional fragment or variant thereof having at least 90% identity to SEQ ID NO. 7.

23. The engineered NK cell of any one of clause 18 to clause 21, wherein the CD79A intracellular signaling domain comprises a polypeptide encoded by a nucleic acid comprising SEQ ID NO: 1 (WT), a polypeptide encoded by a nucleic acid sequence comprising SEQ ID NO: 2 (CD79A (S197A, S203A, T209V), or a functional fragment or variant thereof having at least 90% identity to SEQ ID NO: 1 or SEQ ID NO: 2.

24. The engineered NK cell of any one of clause 18 to clause 21, wherein the CD79B intracellular signaling domain comprises a polypeptide encoded by a nucleic acid comprising SEQ ID NO: 3, or a functional fragment or variant thereof having at least 90% identity to SEQ ID NO: 3.

25. The engineered NK cell of any one of clause 18 to clause 21, wherein the 2B4 intracellular signaling domain comprises a polypeptide encoded by a nucleic acid comprising SEQ ID NO: 4, or a functional fragment or variant thereof having at least 90% identity to SEQ ID NO: 4.

26. The engineered NK cell of any one of clause 18 to clause 21, wherein the CD132 intracellular signaling domain comprises a polypeptide encoded by a nucleic acid comprising SEQ ID NO: 6, or a functional fragment or variant thereof having at least 90% identity to SEQ ID NO: 6.

27. The engineered NK cell of any one of clause 1 to clause 26, wherein the chimeric receptor comprises a CD3e extracellular domain linked to a CD3g extracellular domain, a CD16 transmembrane domain, and 2B4, CD79A, CD79B and CD132 intracellular domains.

28. The engineered NK cell of any one of clause 1 to clause 26, wherein the chimeric receptor comprises a CD3e extracellular domain linked to a CD3g extracellular domain, a CD3e transmembrane domain, and a CD3z intracellular domain.

29. The engineered NK cell of clause 27 or clause 28, wherein the CD3g extracellular domain is linked to a CD3g transmembrane domain, and wherein the CD3g transmembrane domain is linked to a 4-1BB intracellular domain.

30. The engineered NK cell of any one of clause 1 to clause 26, wherein the chimeric receptor comprises a CD3e extracellular domain linked to a CD3d extracellular domain, a CD16 transmembrane domain, and 2B4, CD79A, CD79B and CD132 intracellular domains.

31. The engineered NK cell of any one of clause 1 to clause 30, wherein the chimeric receptor is expressed under the control of a promoter that is transcriptionally active in NK cells.

32. The engineered NK cell of clause 31, wherein the promoter is MND.

33. The engineered NK cell of any one of clause 1 to clause 32, wherein the chimeric receptor comprises a P2A truncated CD34 protein on the terminal end of the chimeric receptor.

34. The engineered NK cell of any one of clause 1 to clause 33, wherein the cell is deficient for NKG2A and/or CD8 expression, activity, or signaling.

35. The engineered NK cell of any one of clause 1 to clause 34, wherein the NK cell is derived from cord blood, peripheral blood, an immortalized cell line, or an iPSC.

36. The engineered NK cell of any one of clause 1 to clause 35, wherein the NK cell is a memory-like NK cell.

37. An engineered T cell comprising a chimeric receptor comprising a CD3 epsilon (CD3e) extracellular domain, a first transmembrane domain, and a first intracellular domain; wherein the first transmembrane domain does not comprise a CD3e transmembrane domain; wherein the engineered T cell does not comprise a T cell receptor (TCR).

38. An engineered T cell comprising a chimeric receptor comprising a CD3 epsilon (CD3e) extracellular domain, a first transmembrane domain, and a first intracellular domain; wherein the first intracellular domain does not comprise a CD3e intracellular domain; wherein the engineered T cell does not comprise a T cell receptor (TCR).

39. The engineered T cell of clause 37 or clause 38, wherein the T Cell Receptor Alpha chain (TRAC) gene is genetically modified or deleted.

40. The engineered T cell of any one of clause 37 to clause 39, wherein the CD3e extracellular domain is noncovalently associated with or linked to a CD3 gamma (CD3g) extracellular domain or a CD3 delta (CD3d) extracellular domain by a linker.

41. The engineered T cell of clause 40, wherein the linker comprises a polypeptide chain comprising a Gly Ser linker.

42. The engineered T cell of clause 40, wherein the linker comprises the polypeptide sequence set forth in SEQ ID NO. 52 or SEQ ID NO. 53.

43. The engineered T cell of any one of clause 37 to clause 42, wherein the CD3e extracellular domain is capable of binding to a T cell engager.

44. The engineered T cell of clause 43, wherein the T cell engager is selected from the list consisting of a bispecific T cell engager (BiTE), a trispecific T cell engager (TriTE), a TeTriTE, and a dual affinity retargeting antibody (DART).

45. The engineered T cell of any one of clause 43 to clause 44, wherein the T cell engager is capable of binding to a target selected from PSMA, MAGE-A4, HER2, GPC3, GD2, FLT3, EPCAM, DLL3, CLDN 18.2, CD38, CD33, CD20, CD19, CD123, BCMA, B&-H7, and Mesothelin.

46. The engineered T cell of any one of clause 43 to clause 45, wherein the CD3e extracellular domain is capable of binding to a bispecific T cell engager (BiTE).

47. The engineered T cell of clause 46, wherein the BiTE is Blinatumomab, Tebentafusp, Mosunetuzumab, Teclistamab, Cibisatamab, or Tarlatamab.

48. The engineered T cell of clause 47, wherein the BiTE is Blinatumomab.

49. The engineered T cell of any one of clause 37 to clause 48, wherein the CD3e extracellular domain comprises a polypeptide encoded by a nucleic acid comprising SEQ ID NO. 23, or a functional fragment or variant thereof having at least 90% identity to SEQ ID 23.

50. The engineered T cell of any one of clause 40 to clause 49, wherein the CD3g extracellular domain or the CD3d extracellular domain is linked to a second transmembrane domain, wherein the second transmembrane domain is linked to a second intracellular domain.

51. The engineered T cell of any one of clause 37 to clause 50, wherein the first transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

52. The engineered T cell of clause 51, wherein the first transmembrane domain comprises a CD3e transmembrane domain.

53. The engineered T cell of any one of clause 50 to clause 52, wherein the second transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

54. The engineered T cell of clause 53, wherein the second transmembrane domain comprises a CD3g transmembrane domain.

55. The engineered T cell of any one of clause 37 to clause 54, wherein the first intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

56. The engineered T cell of clause 55, wherein the first intracellular signaling domain comprises a CD3z ITAM intracellular signaling domain.

57. The engineered T cell of any one of clause 50 to clause 56, wherein the second intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

58. The engineered T cell of clause 57, wherein the second intracellular signaling domain comprises a 4-1BB intracellular signaling domain.

59. The engineered T cell of any one of clause 55 to clause 58, wherein the 4-1BB intracellular signaling domain comprises a polypeptide encoded by a nucleic acid comprising SEQ ID NO. 7, or a functional fragment or variant thereof having at least 90% identity to SEQ ID NO. 7.

60. The engineered T cell of any one of clause 55 to clause 58, wherein the CD79A intracellular signaling domain comprises a polypeptide encoded by a nucleic acid comprising SEQ ID NO: 1 (WT), a polypeptide encoded by a nucleic acid sequence comprising SEQ ID NO: 2 (CD79A (S197A, S203A, T209V), or a functional fragment or variant thereof having at least 90% identity to SEQ ID NO: 1 or SEQ ID NO: 2.

61. The engineered T cell of any one of clause 55 to clause 58, wherein the CD79B intracellular signaling domain comprises a polypeptide encoded by a nucleic acid comprising SEQ ID NO: 3, or a functional fragment or variant thereof having at least 90% identity to SEQ ID NO: 3.

62. The engineered T cell of any one of clause 55 to clause 58, wherein the 2B4 intracellular signaling domain comprises a polypeptide encoded by a nucleic acid comprising SEQ ID NO: 4, or a functional fragment or variant thereof having at least 90% identity to SEQ ID NO: 4.

63. The engineered T cell of any one of clause 55 to clause 58, wherein the CD132 intracellular signaling domain comprises a polypeptide encoded by a nucleic acid comprising SEQ ID NO: 6, or a functional fragment or variant thereof having at least 90% identity to SEQ ID NO: 6.

64. The engineered T cell of any one of clause 37 to clause 63, wherein the chimeric receptor comprises a CD3e extracellular domain linked to a CD3g extracellular domain, a CD16 transmembrane domain, and 2B4, CD79A, CD79B and CD132 intracellular domains.

65. The engineered T cell of any one of clause 37 to clause 63, wherein the chimeric receptor comprises a CD3e extracellular domain linked to a CD3g extracellular domain, a CD3e transmembrane domain, and a CD3z intracellular domain.

66. The engineered T cell of clause 64 or clause 65, wherein the CD3g extracellular domain is linked to a CD3g transmembrane domain, and wherein the CD3g transmembrane domain is linked to a 4-1BB intracellular domain.

67. The engineered T cell of any one of clause 37 to clause 63, wherein the chimeric receptor comprises a CD3e extracellular domain linked to a CD3d extracellular domain, a CD16 transmembrane domain, and 2B4, CD79A, CD79B and CD132 intracellular domains.

68. The engineered T cell of any one of clause 37 to clause 67, wherein the chimeric receptor is expressed under the control of a promoter that is transcriptionally active in T cells.

69. The engineered T cell of clause 68, wherein the promoter is MND.

70. The engineered T cell of any one of clause 37 to clause 69, wherein the chimeric receptor comprises a P2A truncated CD34 protein on the terminal end of the chimeric receptor.

71. The engineered T cell of any one of clause 37 to clause 70, wherein the cell is deficient for NKG2A and/or CD8 expression, activity, or signaling.

72. The engineered T cell of any one of clause 37 to clause 71, wherein the T cell is derived from cord blood, peripheral blood, an immortalized cell line, or an iPSC.

73. The engineered T cell of any one of clause 37 to clause 72, wherein the T cell is a memory T cell.

74. A chimeric receptor capable of being expressed in a natural killer (NK) cell, wherein the receptor comprises:
(a) a CD3 epsilon (CD3e) extracellular domain;
(b) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
(c) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, and IRE1a.

75. A chimeric receptor capable of being expressed in a natural killer (NK) cell, wherein the receptor comprises:
(a) a CD3 epsilon (CD3e) extracellular domain;
(b) a first transmembrane domain selected from the group consisting of: CD3d, CD3e, CD3g, CD16, NKG2D, DAP10, FcγRIIIa, NKp44, NKp30, NKp46, actKIR, NKG2C, CD8α, and IL15; and
(c) a first intracellular signaling domain selected from the group consisting of: CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, and IRE1a.

76. A chimeric receptor capable of being expressed in a natural killer (NK) cell, wherein the receptor comprises:
a CD3 epsilon (CD3e) extracellular domain;
a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

77. A chimeric receptor capable of being expressed in a natural killer (NK) cell, wherein the receptor comprises:
a CD3 epsilon (CD3e) extracellular domain;
a first transmembrane domain selected from the group consisting of: CD3d, CD3e, CD3g, CD16, NKG2D, DAP10, FcγRIIIa, NKp44, NKp30, NKp46, actKIR, NKG2C, CD8α, and IL15; and
a first intracellular signaling domain selected from the group consisting of: CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

78. The chimeric receptor of any one of clauses 74 to 77, wherein the CD3e extracellular domain is noncovalently associated with or linked to a CD3 gamma (CD3g) extracellular domain or a CD3 delta (CD3d) extracellular domain by a linker.

79. The chimeric receptor of clause 78, wherein the linker comprises a polypeptide chain comprising a Gly Ser linker.

80. The chimeric receptor of clause 79, wherein the linker comprises the polypeptide sequence set forth in SEQ ID NO. 52 or SEQ ID NO. 53.

81. The chimeric receptor of any one of clause 74 to clause 80, wherein the CD3e extracellular domain is capable of binding to a bispecific T cell engager (BiTE).

82. The chimeric receptor of clause 81, wherein the BiTE is selected from Blinatumomab, Tebentafusp, Mosunetuzumab, Teclistamab, Cibisatamab, and Tarlatamab.

83. The chimeric receptor of clause 82, wherein the BiTE is Blinatumomab.

84. The chimeric receptor of any one of clause 74 to clause 83, wherein the CD3e extracellular domain comprises a polypeptide encoded by a nucleic acid comprising SEQ ID NO. 23, or a functional fragment or variant thereof having at least 90% identity to SEQ ID NO. 23.

85. The chimeric receptor of any one of clause 74 to clause 84, wherein the first transmembrane domain comprises a CD3e transmembrane domain.

86. The chimeric receptor of any one of clause 78 to clause 85, wherein the CD3g extracellular domain or the CD3d extracellular domain is linked to a second transmembrane domain, wherein the second transmembrane domain is linked to a second intracellular domain.

87. The chimeric receptor of clause 86, wherein the second transmembrane domain is selected from the group consisting of: CD3d, CD3e, CD3g, CD16, NKG2D, DAP10, FcγRIIIa, NKp44, NKp30, NKp46, actKIR, NKG2C, CD8α, and IL15 transmembrane domains.

88. The chimeric receptor of clause 87, wherein the second transmembrane domain comprises a CD3g transmembrane domain.

89. The chimeric receptor of any one of clause 74 to clause 88, wherein the first intracellular signaling domain comprises a CD3z intracellular signaling domain.

90. The chimeric receptor of any one of clause 86 to clause 89, wherein the second intracellular signaling domain is selected from the group consisting of: CD3e, CD3g, 2B4, CD79A, CD79B, CD132, IL2R beta, 4-1BB, FcR gamma ITAM, CD3 zeta ITAM (CD3z ITAM), DNAM-1, NKp80, NTBA, CRACC, CD2, CD27, integrins, IL-15R, IL-18R, IL-12R, IL-21R, and IRE1a intracellular signaling domains.

91. The chimeric receptor of clause 90, wherein second intracellular signaling domain comprises a 4-1BB intracellular signaling domain.

92. The chimeric receptor of any one of clause 74 to clause 91, wherein the 4-1BB intracellular signaling domain comprises a polypeptide encoded by a nucleic acid comprising SEQ ID NO. 7, or a functional fragment or variant thereof having at least 90% identity to SEQ ID NO. 7.

93. The chimeric receptor of any one of clause 74 to clause 91, wherein the CD79A intracellular signaling domain comprises a polypeptide encoded by a nucleic acid comprising SEQ ID NO: 1 (WT), a polypeptide encoded by a nucleic acid sequence comprising SEQ ID NO: 2 (CD79A (S197A, S203A, T209V), or a functional fragment or variant thereof having at least 90% identity to SEQ ID NO: 1 or SEQ ID NO: 2.

94. The chimeric receptor of any one of clause 74 to clause 91, wherein the CD79B intracellular signaling domain comprises a polypeptide encoded by a nucleic acid comprising SEQ ID NO: 3, or a functional fragment or variant thereof having at least 90% identity to SEQ ID NO: 3.

95. The chimeric receptor of any one of clause 74 to clause 91, wherein the 2B4 intracellular signaling domain comprises a polypeptide encoded by a nucleic acid comprising SEQ ID NO: 4, or a functional fragment or variant thereof having at least 90% identity to SEQ ID NO: 4.

96. The chimeric receptor of any one of clause 74 to clause 91, wherein the CD132 intracellular signaling domain comprises a polypeptide encoded by a nucleic acid comprising SEQ ID NO: 6, or a functional fragment or variant thereof having at least 90% identity to SEQ ID NO: 6.

97. The chimeric receptor of any one of clause 74 to clause 96, wherein the chimeric receptor comprises a CD3e extracellular domain linked to a CD3g extracellular domain, a CD16 transmembrane domain, and 2B4, CD79A, CD79B and CD132 intracellular domains.

98. The chimeric receptor of any one of clause 74 to clause 96, wherein the chimeric receptor comprises a CD3e extracellular domain linked to a CD3g extracellular domain, a CD3e transmembrane domain, and a CD3z intracellular domain.

99. The chimeric receptor of clause 97 or clause 98, wherein the CD3g extracellular domain is linked to a CD3g transmembrane domain, and wherein the CD3g transmembrane domain is linked to a 4-1BB intracellular domain.

100. The chimeric receptor of any one of clause 74 to clause 96, wherein the chimeric receptor comprises a CD3e extracellular domain linked to a CD3d extracellular domain, a CD16 transmembrane domain, and 2B4, CD79A, CD79B and CD132 intracellular domains.

101. The chimeric receptor of any one of clause 74 to clause 100, wherein the chimeric receptor is expressed under the control of a promoter that is transcriptionally active in NK cells.

102. The chimeric receptor of clause 101, wherein the promoter is MND.

103. The chimeric receptor of any one of clause 74 to clause 102, wherein the chimeric receptor comprises a P2A truncated CD34 protein on the terminal end of the chimeric receptor.

104. A vector comprising a nucleic acid encoding the chimeric receptor of any one of clause 74 to clause 103.

105. The vector of clause 104, wherein the vector is a viral vector.

106. The vector of clause 104 or clause 105, wherein the viral vector is a retroviral or lentiviral vector.

107. An engineered NK cell comprising (i) the chimeric receptor of any one of clause 74 to clause 103, and/or (ii) the vector according to any one of clause 104 to clause 106.

108. The engineered NK cell of clause 107, wherein the cell is deficient for NKG2A and/or CD8 expression, activity, or signaling.

109. The engineered NK cell of any one of clause 107 to clause 108, wherein the NK cell is derived from cord blood, peripheral blood, an immortalized cell line, or an iPSC.

110. The engineered NK cell of any one of clause 107 to clause 109, wherein the NK cell is a memory-like NK cell.

111. The engineered NK cell of any one of clause 107 to clause 110, wherein the extracellular domain is capable of binding to a T cell engager.

112. The engineered NK cell of clause 111, wherein the T cell engager is selected from the list consisting of a bispecific T cell engager (BiTE), a trispecific T cell engager (TriTE), a TeTriTE, and a dual affinity retargeting antibody (DART).

113. An engineered T cell comprising (i) the chimeric receptor of any one of clause 74 to clause 103, and/or (ii) the vector according to any one of clause 104 to clause 106.

114. The engineered T cell of clause 113, wherein the engineered T cell does not comprise a T cell receptor (TCR).

115. The engineered T cell of clause 113 or clause 114, wherein the T Cell Receptor Alpha chain (TRAC) gene is genetically modified or deleted.

116. The engineered T cell of any one of clause 113 to clause 115, wherein the cell is deficient for NKG2A and/or CD8 expression, activity, or signaling.

117. The engineered T cell of any one of clause 113 to clause 116, wherein the T cell is derived from cord blood, peripheral blood, an immortalized cell line, or an iPSC.

118. The engineered T cell of any one of clause 113 to clause 117, wherein the T cell is a memory T cell.

119. The engineered T cell of any one of clause 113 to clause 118, wherein the extracellular domain is capable of binding to a T cell engager.

120. The engineered T cell of clause 119, wherein the T cell engager is selected from the list consisting of a bispecific T cell engager (BiTE), a trispecific T cell engager (TriTE), a TeTriTE, and a dual affinity retargeting antibody (DART).

121. A bicistronic chimeric receptor comprising a first and a second chimeric receptor, wherein the first and the second chimeric receptors are co-expressed by a bicistronic vector, wherein the first chimeric receptor is the chimeric receptor of any one of clause 74 to clause 103.

122. A bicistronic chimeric receptor comprising a first and a second chimeric receptor, wherein the first and the second chimeric receptors are co-expressed by a bicistronic vector, wherein the first chimeric receptor comprises a CD3e extracellular domain; and wherein the second chimeric receptor comprises a CD3g or a CD3d extracellular domain.

123. The bicistronic chimeric receptor of clause 121 or clause 122, wherein the first chimeric receptor comprises a CD3e extracellular domain, a CD3e transmembrane domain, and a CD3e intracellular domain; wherein the second chimeric receptor comprises a CD3g extracellular domain, a CD3g transmembrane domain, and a CD3g intracellular domain.

124. The bicistronic chimeric receptor of clause 121, wherein the first chimeric receptor comprises a CD3e extracellular domain, a CD3e transmembrane domain, and CD79A and 2B4 intracellular domains; wherein the second chimeric receptor comprises a CD3g extracellular domain, a CD3g transmembrane domain, and a CD79B intracellular domain.

125. The bicistronic chimeric receptor of clause 121 or clause 122, wherein the first chimeric receptor comprises a CD3e extracellular domain, a CD3e transmembrane domain, and CD79A, CD132 and 2B4 intracellular domains; wherein the second chimeric receptor comprises a CD3g extracellular domain, a CD3g transmembrane domain, and CD79B and IL2Rbeta intracellular domains.

126. The bicistronic chimeric receptor of clause 121 or clause 122, wherein the first chimeric receptor comprises a CD3e extracellular domain, a CD3e transmembrane domain, and CD79A, CD132 and 2B4 intracellular domains; wherein the second chimeric receptor comprises a CD3d extracellular domain, a CD3d transmembrane domain, and CD79B and IL2Rbeta intracellular domains.

127. The bicistronic chimeric receptor of clause 121 or clause 122, wherein the first chimeric receptor comprises a CD3e extracellular domain, a CD3e transmembrane domain, and a CD3zeta intracellular domain; wherein the second chimeric receptor comprises a CD3g extracellular domain, a CD3g transmembrane domain, and a 41BB intracellular domain.

128. The bicistronic chimeric receptor of clause 121 or clause 122, wherein the first chimeric receptor comprises a CD3e extracellular domain, a CD3e transmembrane domain, and CD3zeta and CD132 intracellular domains; wherein the second chimeric receptor comprises a CD3g extracellular domain, a CD3g transmembrane domain, and 41BB and IL2Rb intracellular domains.

129. The bicistronic chimeric receptor of clause 121 or clause 122, wherein the first chimeric receptor comprises:
(a) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

130. The bicistronic chimeric receptor of clause 121 or clause 122, wherein the first chimeric receptor comprises:
(a) a first transmembrane domain selected from the group consisting of: CD3e, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3d, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

131. The bicistronic chimeric receptor of clause 121 or clause 122, wherein the first chimeric receptor comprises a CD3e extracellular domain, a CD3e transmembrane domain, and a CD3zeta intracellular domain; and wherein the second chimeric receptor comprises a CD3g extracellular domain, a CD3g transmembrane domain, and a 41BB intracellular domain.

132. The bicistronic chimeric receptor of clause 121 or clause 122, wherein the first chimeric receptor comprises a CD3e extracellular domain, a CD3e transmembrane domain, and CD3zeta and 2B4 intracellular domains; and wherein the second chimeric receptor comprises a CD3g extracellular domain, a CD3g transmembrane domain, and a OX40 intracellular domain.

133. The bicistronic chimeric receptor of any one of clause 121 to clause 132, wherein the bicistronic chimeric receptor is expressed under the control of a promoter that is transcriptionally active in NK cells.

134. The bicistronic chimeric receptor of clause 133, wherein the promoter is MND.

135. The bicistronic chimeric receptor of any one of clause 121 to clause 134, comprising a P2A truncated CD34 protein on the terminal end of the chimeric receptor.

136. A bicistronic vector comprising a nucleic acid encoding the bicistronic chimeric receptor of any one of clause 121 to clause 135.

137. The bicistronic vector of clause 136, wherein the bicistronic vector is a viral vector.

138. The bicistronic vector of clause 137, wherein the viral vector is a retroviral or lentiviral vector.

139. A tricistronic chimeric receptor comprising a first, a second, and a third chimeric receptor, wherein the first, the second, and the third chimeric receptors are co-expressed by a tricistronic vector, wherein the first chimeric receptor comprises a CD3e extracellular domain; wherein the second chimeric receptor comprises a CD3g extracellular domain; and wherein the third chimeric receptor comprises a CD3d extracellular domain.

140. The tricistronic chimeric receptor of clause 139, wherein the first chimeric receptor comprises:
(a) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

141. The tricistronic chimeric receptor of clause 139, wherein the first chimeric receptor comprises:
(a) a first transmembrane domain selected from the group consisting of: CD3e, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3d, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

142. The tricistronic chimeric receptor of clause 139, wherein the first chimeric receptor comprises a CD3e extracellular domain, a CD3e transmembrane domain, and a CD3z intracellular domain; wherein the second chimeric receptor comprises a CD3d extracellular domain, a CD3d transmembrane domain, and a 2B4 intracellular domain; and wherein the third chimeric receptor comprises a CD3g extracellular domain, a CD3g transmembrane domain, and a 4-1BB intracellular domain.

143. A tricistronic vector comprising a nucleic acid encoding the tricistronic chimeric receptor of any one of clause 139 to clause 142.

144. The tricistronic vector of clause 143, wherein the tricistronic vector is a viral vector.

145. The tricistronic vector of clause 144, wherein the viral vector is a retroviral or lentiviral vector.

146. An engineered NK cell comprising (i) the bicistronic chimeric receptor of any one of clause 121 to clause 135, and/or (ii) the bicistronic vector according to any one of clause 136 to clause 138.

147. An engineered NK cell comprising (i) the tricistronic chimeric receptor of any one of clause 139 to clause 142, and/or (ii) the tricistronic vector according to any one of clause 143 to clause 145.

148. The engineered NK cell of clause 146 or 147, wherein the cell is deficient for NKG2A and/or CD8 expression, activity, or signaling.

149. The engineered NK cell of any one of clause 146 to clause 148, wherein the NK cell is derived from cord blood, peripheral blood, an immortalized cell line, or an iPSC.

150. The engineered NK cell of any one of clause 146 to clause 149, wherein the NK cell is a memory-like NK cell.

151. An engineered T cell comprising (i) the bicistronic chimeric receptor of any one of clause 121 to clause 135, and/or (ii) the bicistronic vector according to any one of clause 136 to clause 138.

152. An engineered T cell comprising (i) the tricistronic chimeric receptor of any one of clause 139 to clause 142, and/or (ii) the tricistronic vector according to any one of clause 143 to clause 145.

153. The engineered T cell of clause 151 or 152, wherein the engineered T cell does not comprise a T cell receptor (TCR).

154. The engineered T cell of clause 151 or clause 153, wherein the T Cell Receptor Alpha chain (TRAC) gene is genetically modified or deleted.

155. The engineered T cell of any one of clause 151 to clause 154, wherein the cell is deficient for NKG2A and/or CD8 expression, activity, or signaling.

156. The engineered T cell of any one of clause 151 to clause 155, wherein the T cell is derived from cord blood, peripheral blood, an immortalized cell line, or an iPSC.

157. The engineered T cell of any one of clause 151 to clause 156, wherein the T cell is a memory T cell.

158. A method of inducing an immune response to a disease in a subject in need thereof comprising administering to the subject the engineered NK cell of any one of clause 1 to clause 36, clause 107 to clause 112, and clause 146 to clause 150.

159. A method of inducing an immune response to a disease in a subject in need thereof comprising administering to the subject the engineered T cell of any one of clause 37 to clause 73, clause 113 to clause 120, and clause 151 to clause 157.

160. The method of clause 158 or clause 159, comprising administering to the subject a T cell engager.

161. The method of clause 160, comprising administering to the subject a BiTE.

162. The method of clause 161, wherein the BiTE is selected from Blinatumomab, Tebentafusp, Mosunetuzumab, Teclistamab, Cibisatamab, and Tarlatamab.

163. The method of clause 162, wherein the BiTE is Blinatumomab.

164. The method of any one of clause 160 to clause 163, wherein the T cell engager is capable of binding to a target selected from PSMA, MAGE-A4, HER2, GPC3, GD2, FLT3, EPCAM, DLL3, CLDN 18.2, CD38, CD33, CD20, CD19, CD123, BCMA, B&-H7, and Mesothelin.

165. The method of any one of clause 158 to clause 164, wherein the disease is cancer, an autoimmune condition, or an infectious disease.

166. The method of clause 165, wherein the disease is cancer.

167. The method of clause 166, wherein the cancer comprises a solid tumor.

168. The method of clause 166, wherein the cancer is a hematologic cancer.

169. The method of clause 166, wherein the cancer is colorectal cancer.

170. The method of clause 166, wherein the cancer is small cell lung cancer.

171. The method of any one of clause 158 to clause 170, wherein Graft Versus Host Disease (GVHD) is not induced in the subject.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: CD3 Chimeric Receptor Expressing NK Cells Enable New Strategies to Target Different Cancer with Reduced Toxicity

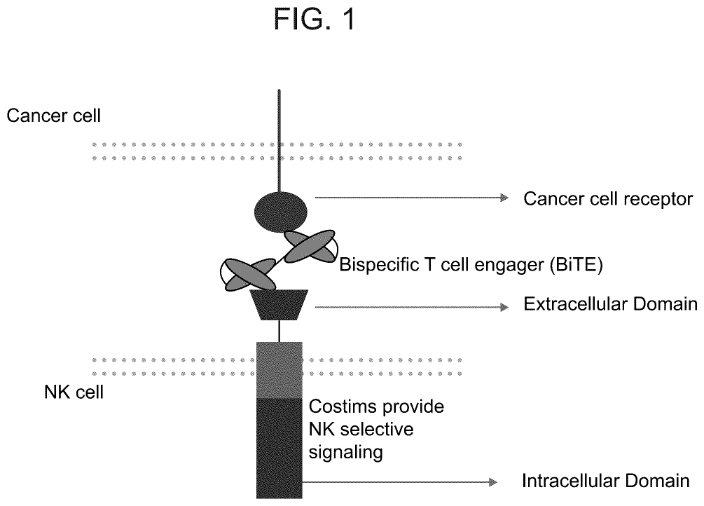

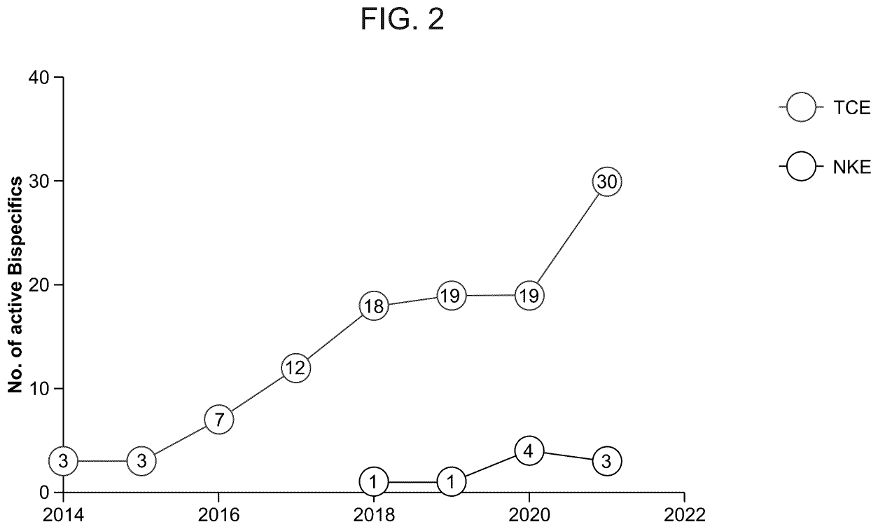

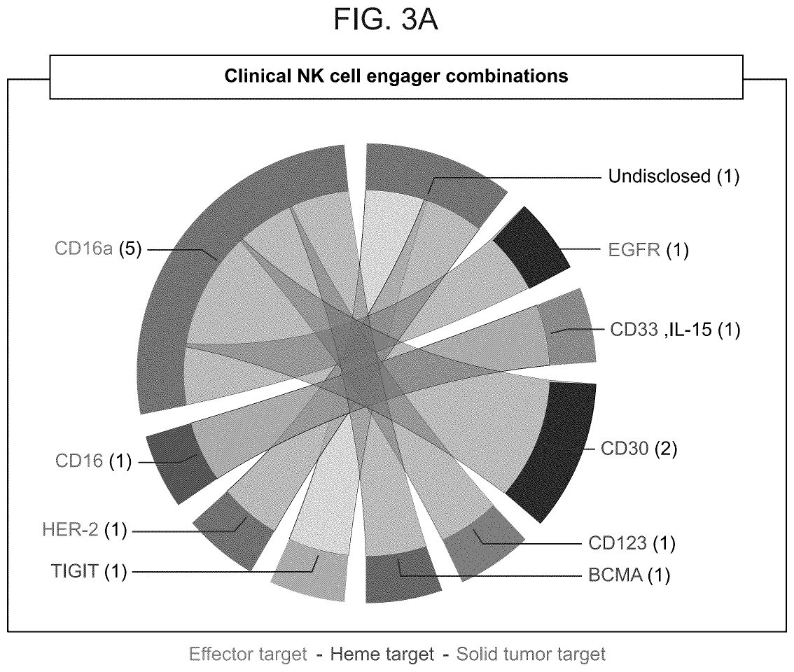

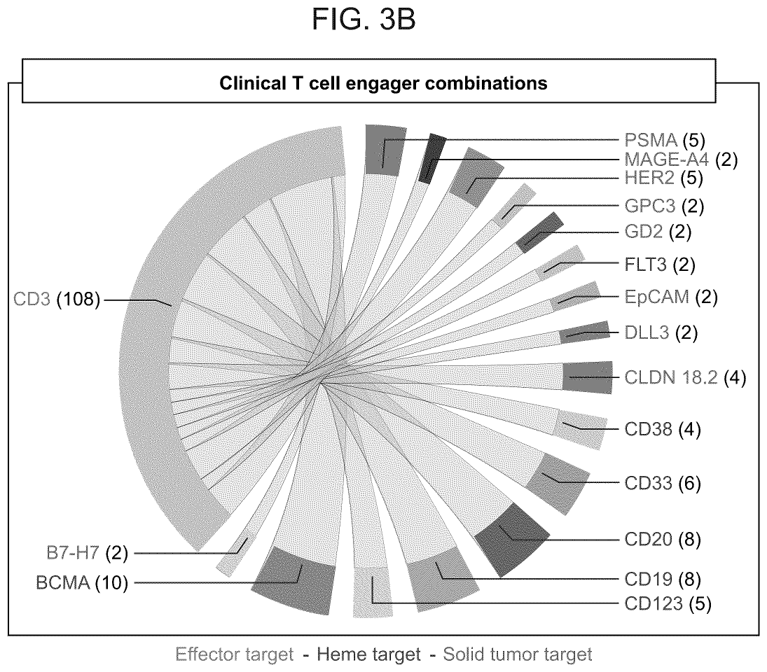

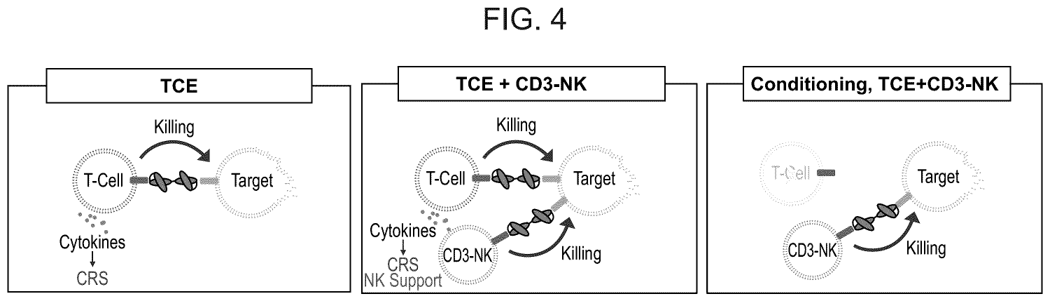

FIG. 1 shows different strategies for using NK cells to target cancer. CD3 chimeric receptor expressing NK cells (CD3-NK cells) would enable use of bispecific T cell engagers (BiTEs) to direct CD3-NK cell killing of cancer cells. The combination of CD3-NK cells would allow for use of NK cells in novel indications with target flexibility. Additional, use of a CD3 chimeric receptor with improved intracellular domains would provide signaling selective to NK cells, improving function. While there are NK cell engagers in development, FIG. 2, FIG. 3 and FIG. 4 illustrate that T cell engagers (TCEs), such as BiTEs, dominate the retargeting landscape. FIG. 2 shows the number of bispecific TCEs far outnumber that of NK cell engagers (NKEs). Additionally, FIG. 3A shows that NKEs are not only few in numbers but limited in tumor target antigens while FIG. 3B shows that TCEs are plentiful and cover a diverse tumor antigen target repertoire. Moreover, as shown in FIG. 4, TCEs activate endogenous T cells and pose a high risk of cytokine release syndrome (CRS) and might not overcome the suppressive tumor microenvironment (TME). CD3-NK cells, used in combination with TCEs, would enable a coordinated antitumor attack and increase anti-tumor activity and help in overcoming the suppressive TME. Alternatively, a conditioning regimen to deplete endogenous T cells would minimize risk of CRS while engineered CD3-NK cells used in combination with TCEs can still effectively kill tumor cells.

### Example 2: Stable Cell Surface Expression of CD3e Containing Receptors in NK Cells

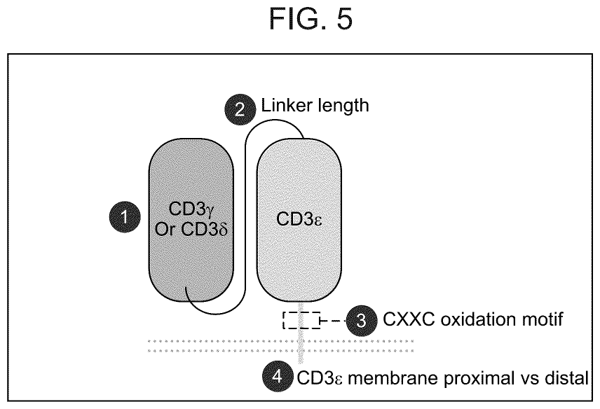

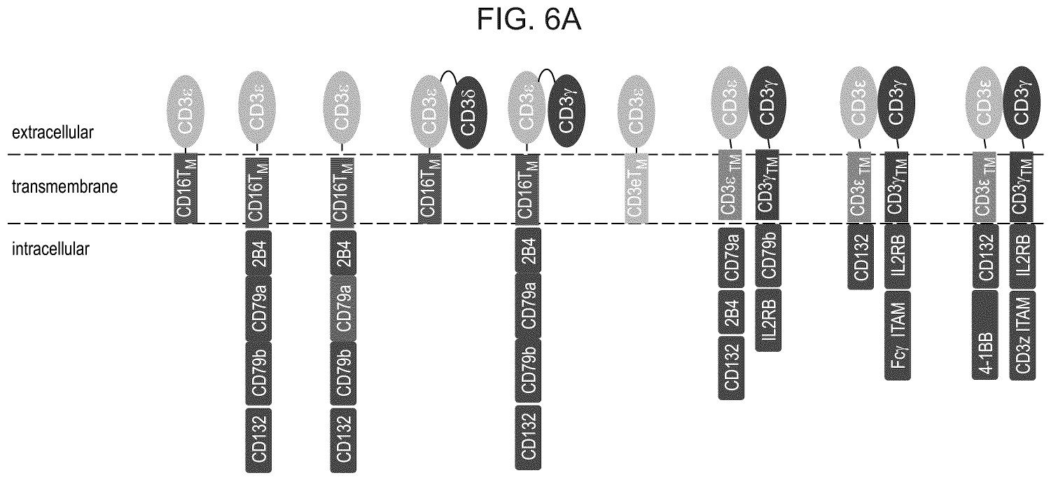

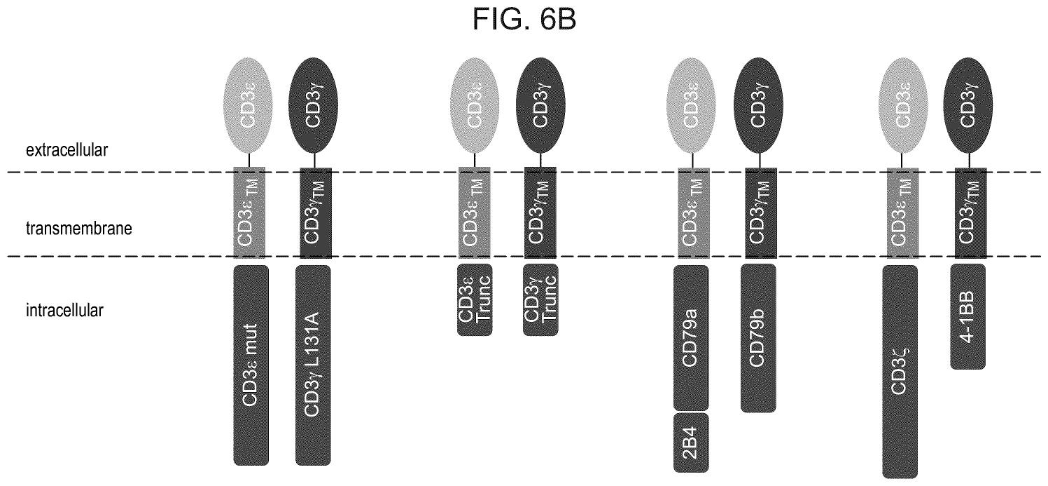

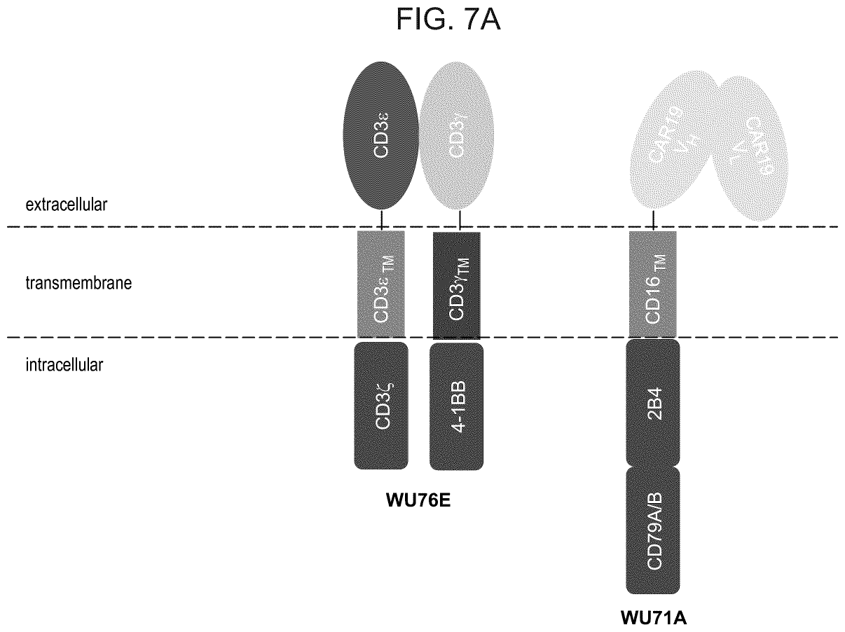

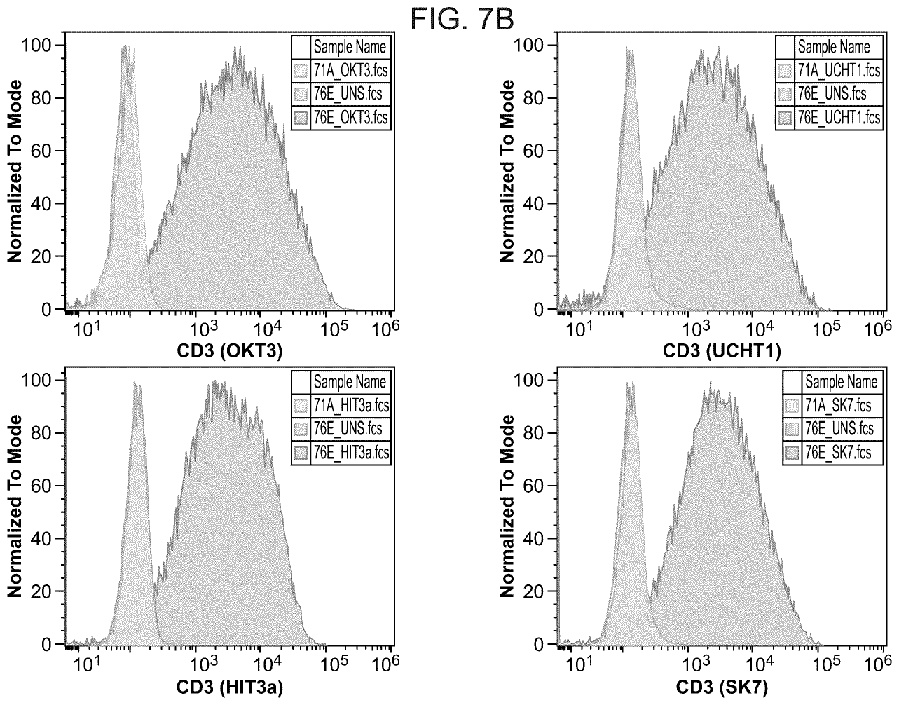

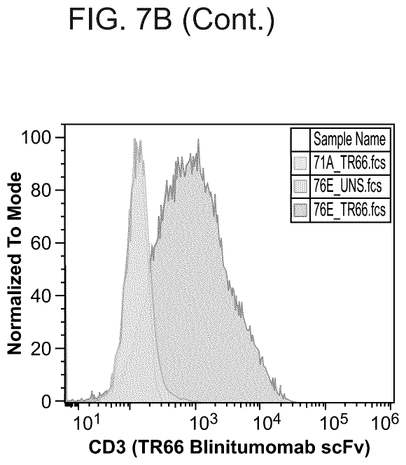

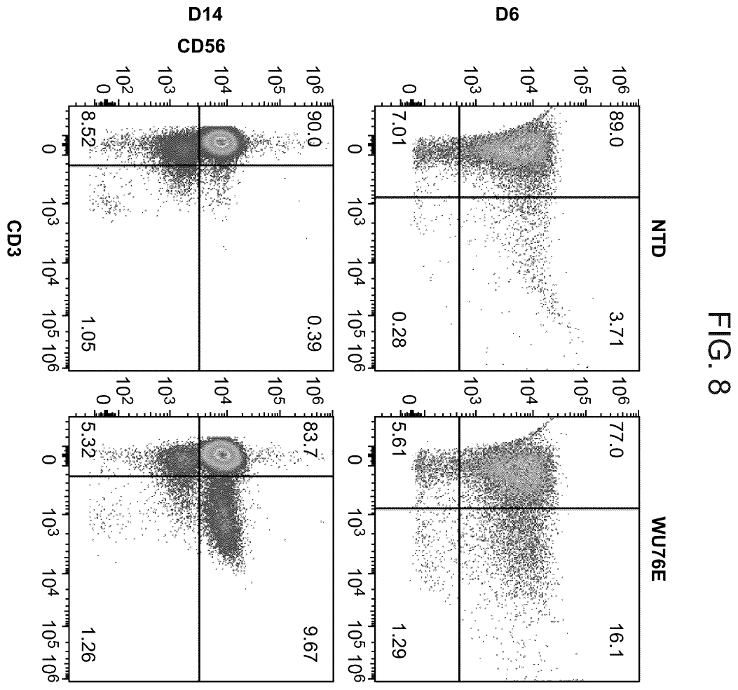

Many BiTEs target the CD3e chain of the CD3-TCR complex, but CD3e typically cannot traffic to the NK cell surface on its own. FIG. 5 shows a strategy for stabilizing cell surface expression of CD3e as a single chain variable fragment (Fv) by co-expression with other CD3 members through a linker and through altering the highly conserved CXXC motif found on the membrane proximal end of CD3e, CD3g or CD3d and through varying the position of CD3e as the membrane proximal or distal domain within the extracellular region. Utilizing the aforementioned strategies, several illustrative chimeric receptor and bicistronic chimeric receptor constructs for expression of CD3e on the cell surface are shown in FIG. 6A and FIG. 6B. To generate CD3-NK cells, NK cells were isolated using STEMCELL kits and primed on day 0. Exogenous factors were then added to further expand the NK cell culture on day 1. On day 3, NK cells were transduced with lentivirus plasmid constructs expressing the desired chimeric receptor through an 8 hour co-culture in a cell culture vessel. Given that CD3e cannot traffic to the cell membrane on its own, experiments were performed to assess the feasibility of expressing CD3e on the cell surface. 293T-X cells were transfected with lentivirus plasmid constructs expressing the chimeric receptors WU76E or WU71A shown in FIG. 7A. Forty-eight hours post transfection, 293T-X cells were stained using 1 of 5 anti-CD3 antibody clones that target CD3e: OKT3, UCHT1, TR66, HIT3a, or SK7 and assessed for cell surface expression of CD3 by flow cytometry. As shown in FIG. 7B, CD3e expression was detected on 293T-X cells expressing the WU76E construct comprising a CD3e extracellular domain but not on the cells expressing the negative control WU71A construct comprising a CD19 extracellular domain.

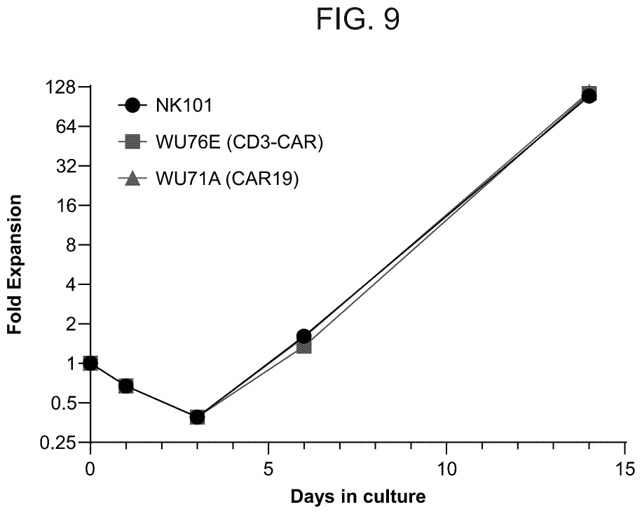

To assess expression of CD3e on human NK cells, non-transduced NK cells (NTD), or NK cells virally transduced with the WU76E CD3e expressing construct (WU76E-NK cells) were stained with anti-CD3e and anti-CD56 antibodies on day 6 or day 14 of manufacturing. Percent of cells with cell surface expression of CD3e and CD56 was quantified by flow cytometry. FIG. 8 shows stable CD3e expression on the cell surface of NK cells transduced with WU76E and FIG. 9 shows that NK cells transduced with WU76E expanded in culture similarly to non-transduced NK cells (NK101) and NK cells transduced with WU71A (WU71A-NK cells). Taken together, Example 1 demonstrates that CD3e can traffic and be expressed on the cell surface of NK cells in the absence of the complete TCR complex. Furthermore, the expression of CD3 on the cell surface did not inhibit NK cell growth.

### Example 3: NK Cells Expressing CD3e Chimeric Receptors Display Potent Cytotoxicity Against Tumor Cells in Combination with a BiTE

Experiments were performed to assess the ability of WU76E NK cells to kill tumor cells in the presence of Blinatumomab, a BiTE which targets CD19 and CD3.

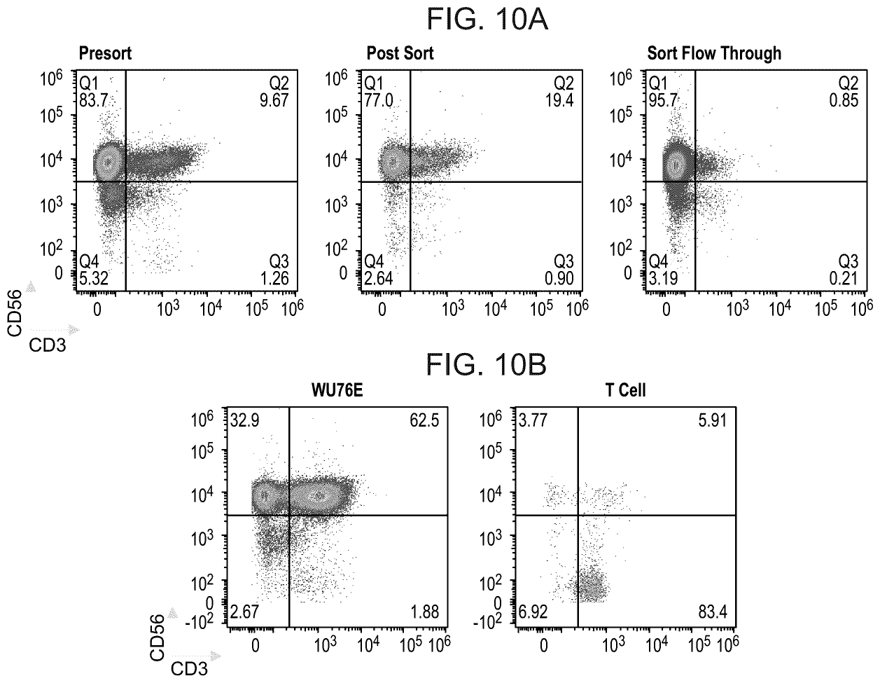

STEMCELL Human CD3 Positive Selection kits were used to positively select for CD3+WU76E-NK cells on day 13 of manufacturing. Cell surface expression of CD56 and CD3 on WU76E-NK cells were quantified by flow cytometry on three populations: WU76E-NK cells prior to CD3 positive selection (Presort), WU76E-NK cells that were positively selected (Post Sort) and the flow through population that was not selected. As shown in FIG. 10A, purity of CD3+WU76E-NK cells immediately post cell enrichment appeared low at 19.4% due to epitope blocking by the CD3 selection antibody cocktail, which dampened the binding of the anti-CD3 antibody used for flow cytometry staining. However, using T cells as a negative staining control, FIG. 10B shows CD3+WU76E-NK cells were enriched after the effects of epitope blocking waned 24 hours after positive selection.

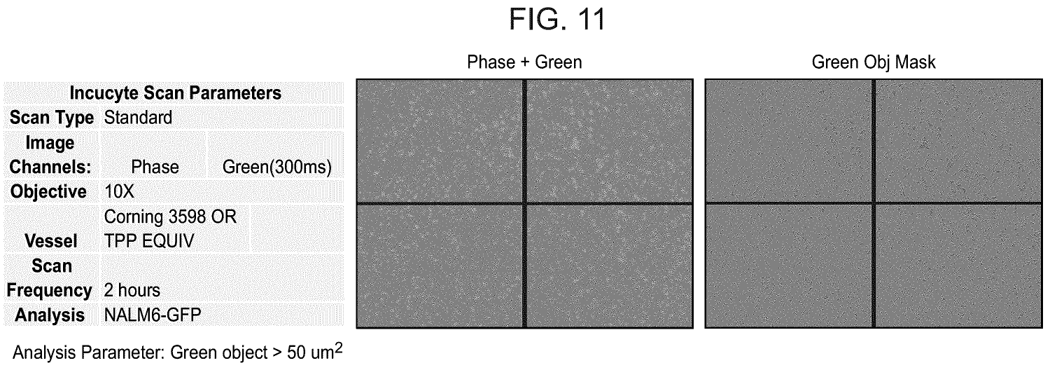

To test the cytotoxicity of CD3+WU76E NK cells in combination with Blinatumomab, GFP+NALM6 cancer cells expressing CD19 were selected as targets for killing. Cells were cultured and imaged with a Sartorius Incucyte using the 10× objective every two hours. Green objects greater than 50 μm² in size were identified as NALM6-GFP cells and quantified. FIG. 11 shows the actual phase and green signal (GFP) images captured and the green object mask analysis done by the Sartorius Incucyte software to discern viable NALM6 cells.

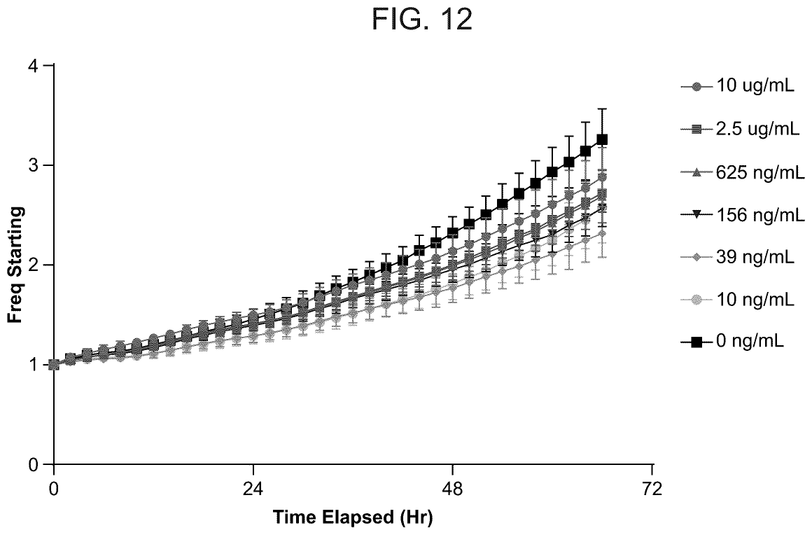

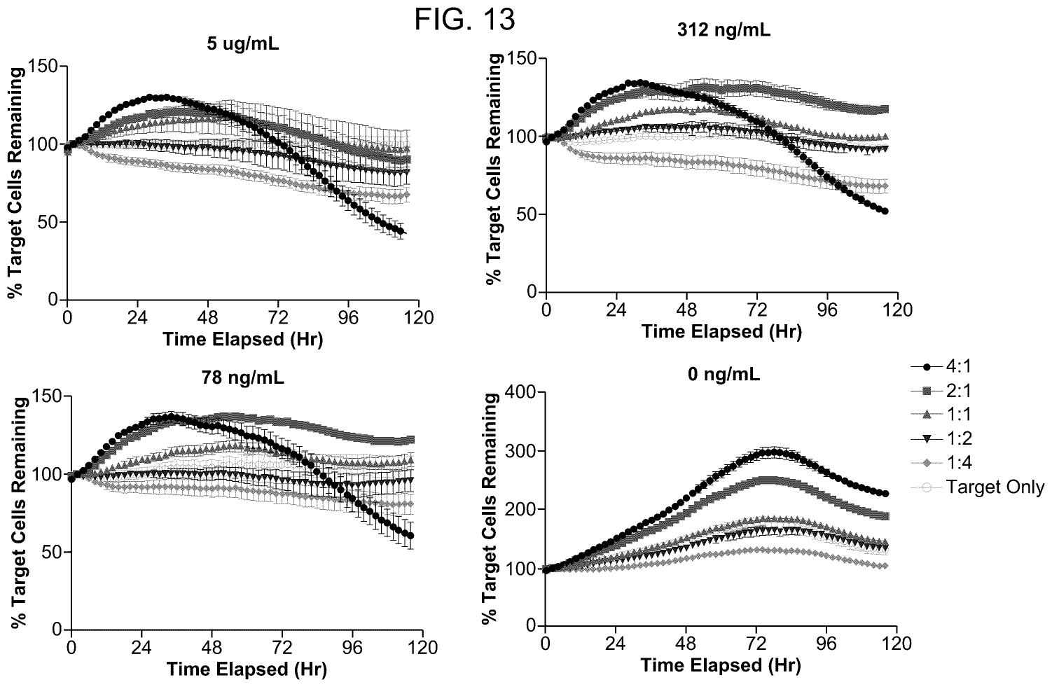

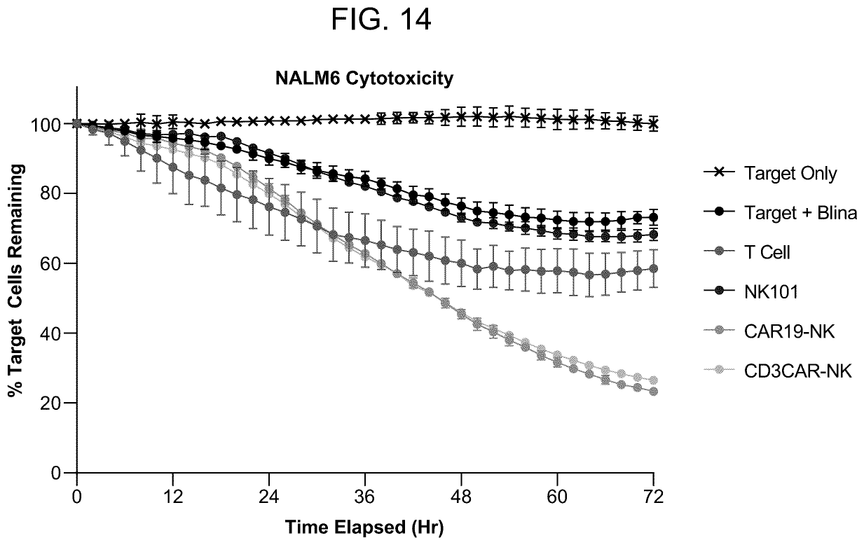

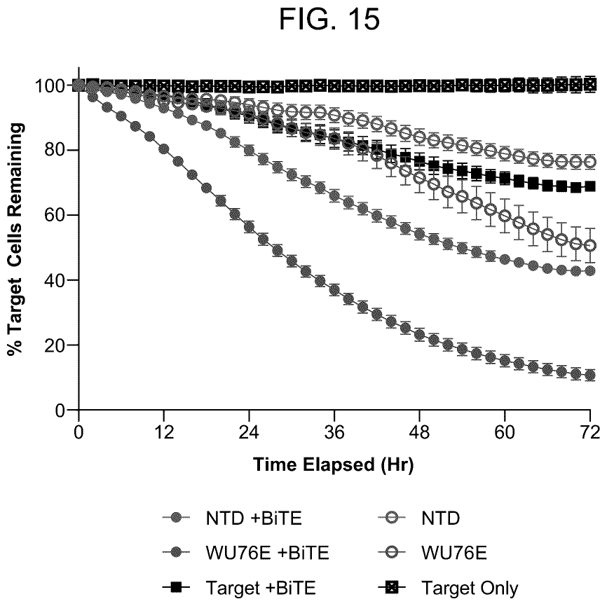

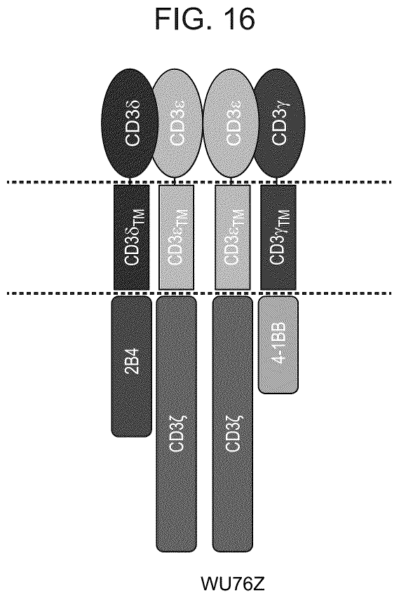

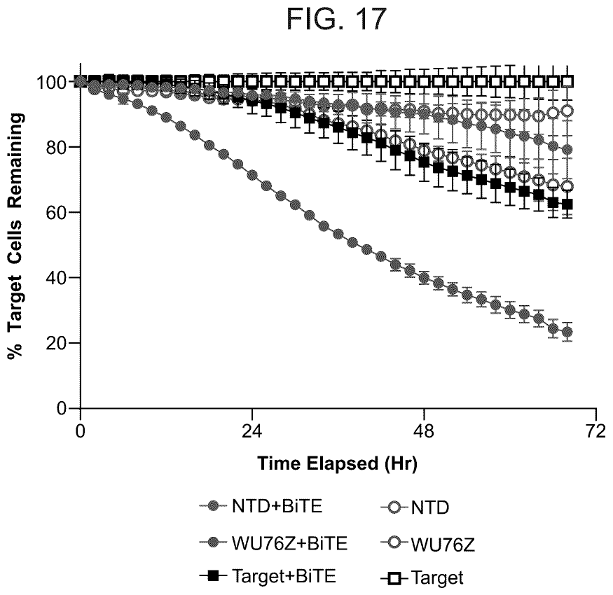

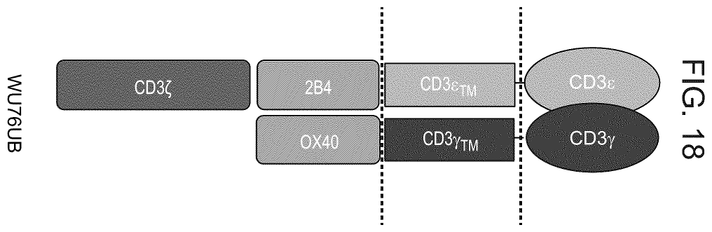

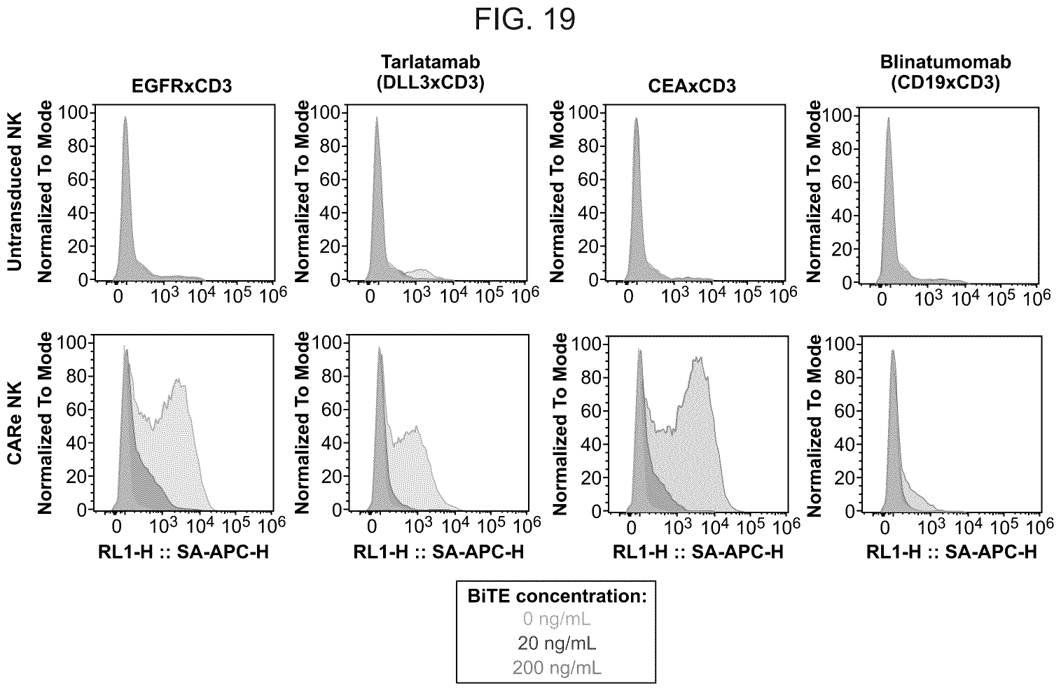

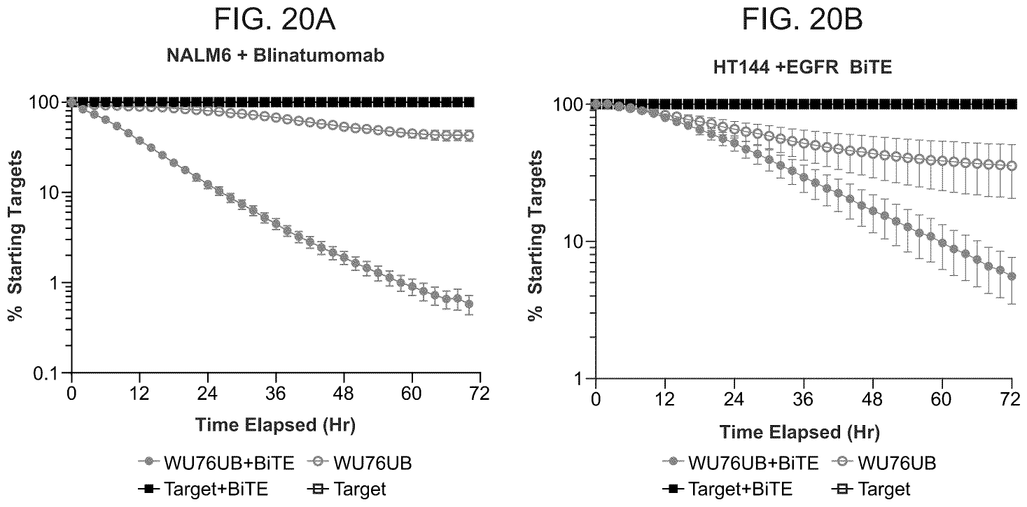

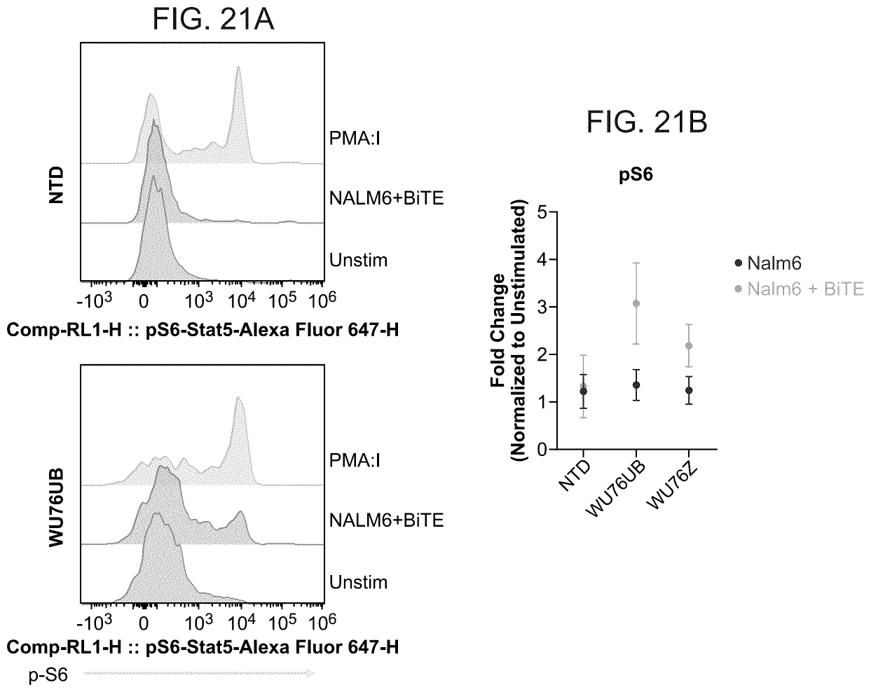

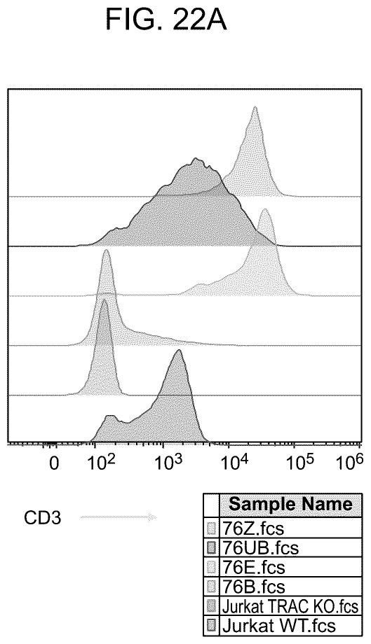

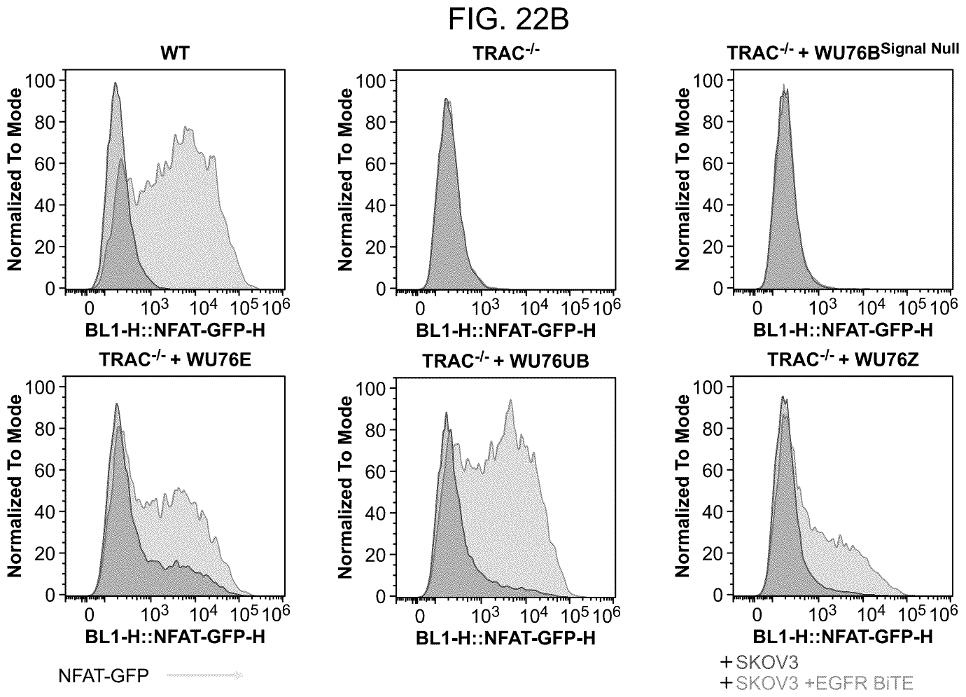

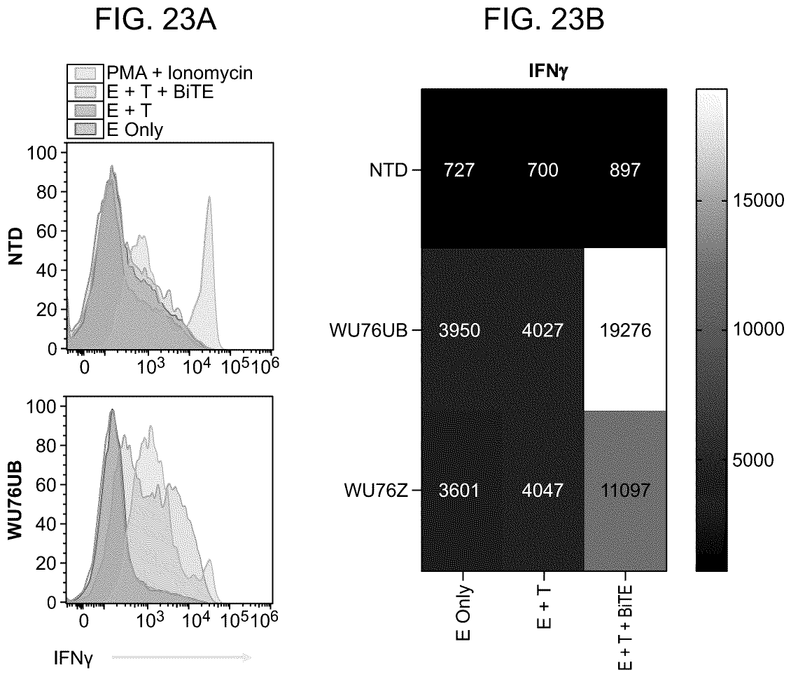

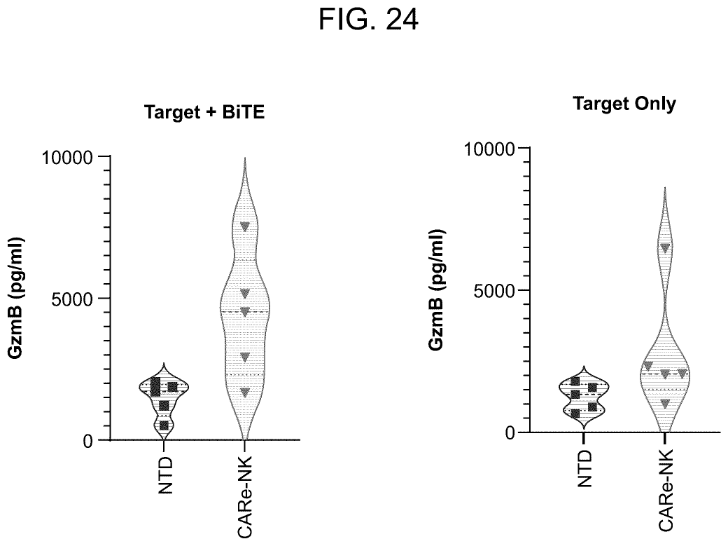

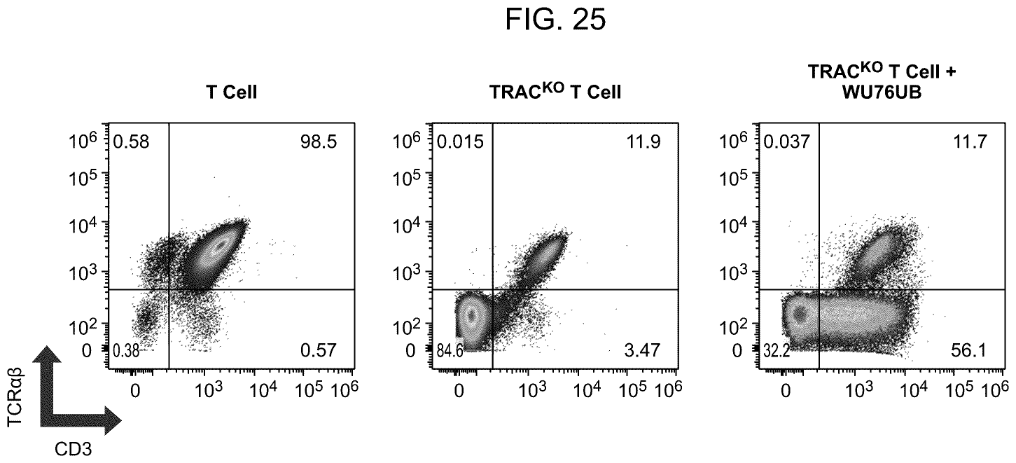

NALM6 cells were cultured in the presence of different concentrations of Blinatumomab in complete RPMI media to determine the effects of Blinatumomab alone on cell growth and identify a suitable concentration for use with WU76E-NK cells. Cell growth was measured via the Sartorius Incucyte and normalized to the number of starting cells (2×10⁵ cells/well). As shown in FIG. 12, there were small decreases in cell growth across the tested blinatumomab concentrations but the NALM6 cells still expanded. NALM6 cells were then co-cultured with different ratios of naïve T cells in the presence of 5 g/mL, 312 ng/mL, 78 ng/mL or Ong/mL of Blinatumomab and growth of NALM6 cells were measured via the Sartorius Incucyte and normalized to the number of starting cells (2×10⁵ cells/well). FIG. 13 shows that while naïve T cells alone cannot kill NALM6 cells, they do kill NALM6 cells in the presence of Blinatumomab, evident by the drop in percent of target cells remaining after 72 hours of co-culturing at the 4:1 effector to target ratio across all concentrations of Blinatumomab.

Lastly, isolated NK cells that were primed, expanded, and transduced with lentivirus expressing WU76E as described in example 2 and subsequently positively selected for CD3 on day 14 of culturing as described earlier in this example were used on day 15 for cytotoxicity assays. Briefly, 2×10⁴ NALM6 cells per well were cultured in six different conditions:
(1) target only—NALM6 cells alone,
(2) target+Blina, NALM6 cells with 100 ng/mL of Blinatumomab,
(3) T Cell—NALM6 cells co-cultured with naïve, isolated CD3+ T cells at a 1:3 effector to target ratio in the presence of 100 ng/mL of Blinatumomab,
(4) NK101—NALM6 cells co-cultured with NK101 at a 1:3 effector to target ratio in the presence of 100 ng/mL of Blinatumomab,
(5) CAR19-NK—NALM6 cells co-cultured with WU71A-NK cells at a 1:3 effector to target ratio in the presence of 100 ng/mL of Blinatumomab, and
(6) CD3CAR-NK—NALM6 cells co-cultured with WU76E-NK cells at a 1:3 effector to target ratio in the presence of 100 ng/mL of Blinatumomab.

All T and NK cells were isolated from the same donor. As shown in FIG. 14, WU76E-NK cells were effective at killing NALM6 cells in the presence of 100 ng/mL of Blinatumomab at a 1:3 E:T ratio and their cytotoxicity activity was comparable to that of WU71A-NK cells expressing a chimeric receptor directly targeting CD19.

WU76E-NK cells were further tested in another set of experiments with the following conditions:
(1) Target only—NALM6 cells alone
(2) Target+BiTE—NALM6 cells with 200 ng/mL of Blinatumomab
(3) NTD—NALM6 cells co-cultured with untransduced NK cells (NTD)
(4) NTD+BiTE—NALM6 cells co-cultured with untransduced NK cells at a 1:1 effector to target ratio in the presence of 200 ng/mL of Blinatumomab
(5) WU76E—NALM6 cells co-cultured with WU76E-NK cells at a 1:1 effector to target ratio
(6) WU76E+BiTE—NALM6 cells co-cultured with WU76E-NK cells at a 1:1 effector to target ratio in the presence of 200 ng/mL of Blinatumomab

As shown in FIG. 15, WU76E-NK cells were effective at killing NALM6 cells at a 1:1 E:T ratio and in the presence of 200 ng/mL of Blinatumomab. Collectively, these results show that NK cells transduced with CD3e constructs work well with a commercially available BiTE in killing cancer cells.

### Example 4: NK Cells Expressing a Tricistronic CD3e Chimeric Receptor Display Potent Cytotoxicity Against Tumor Cells in Combination with a BiTE

Experiments were performed to assess the ability of WU76Z-NK cells to kill tumor cells in the presence of Blinatumomab.

As shown in FIG. 16, WU76Z is a tricistronic construct encoding three CD3 chains. The extracellular, transmembrane, and intracellular signaling domains for the first chain are CD3e, CD3e, and CD3z, respectively. Those for the second chain are CD3d CD3d, and 2B4, respectively. Those for the third chain are CD3g, CD3g, and 41BB, respectively. NK cells were prepared and transduced with WU76Z construct as described in the preceding examples and tested in the following coculture conditions:
(1) Target—NALM6 cells alone
(2) Target+BiTE—NALM6 cells with 200 ng/mL of Blinatumomab
(3) NTD—NALM6 cells co-cultured with untransduced NK cells
(4) NTD+BiTE—NALM6 cells co-cultured with untransduced NK cells at a 1:1 effector to target ratio in the presence of 200 ng/mL of Blinatumomab
(5) WU76Z—NALM6 cells co-cultured with WU76Z-NK cells at a 1:1 effector to target ratio
(6) WU76Z+BiTE—NALM6 cells co-cultured with WU76Z-NK cells at a 1:1 effector to target ratio in the presence of 200 ng/mL of Blinatumomab

As shown in FIG. 17, WU76Z-NK cells were effective at killing NALM6 cells at a 1:1 E:T ratio and in the presence of 200 ng/mL of Blinatumomab. These results show that tricistronic constructs are stably expressed on the cell surface of NK cells and mediate tumor cell killing in the presence of commercially available BiTEs.

### Example 5: CD3e Chimeric Receptor Constructs with Varying Intracellular Domains are all Capable of Conferring T Cell Engager Specific Activity to NK Cells

Experiments were performed to assess the ability of NK cells with a chimeric CD3e receptor with different intracellular signaling domains to kill tumor cells in the presence of Blinatumomab.

As shown in FIG. 18, the WU76UB construct differs from the WU76E construct in its intracellular signaling domains. The intracellular signaling domains of the CD3e chain are 2B4 and CD3z while that of the CD3g chain is OX40. NK cells were prepared and transduced with the WU76UB construct as described in the preceding examples. To confirm that the CD3 chains expressed on the NK cell surface are capable of binding commercially available T cell engagers, untransduced NK cells and WU76UB-NK cells were incubated with His-tagged biosimilars for blinatumomab and tarlatamab, as well as EGFR×CD3 and CEA×CD3 T cell engagers at two different concentrations (20 ng/ml and 200 ng/ml). Binding of the T cell engagers was detected using an anti-His antibody and quantified by flow cytometry. As shown in FIG. 19, there was no binding of any of the T cell engagers to untransduced NK cells. At 20 ng/mL, both EGFR×CD3 and CEA×CD3 T cell engagers binding to WU76UB-NK cells was detected by flow cytometry.

At 200 ng/mL, binding to WU76UB-NK cells was detected for all four T cell engagers. The cytotoxicity activity of WU76UB-NK cells against NALM6 and HT144 target cells were then tested in the following conditions:
(1) Target—target cells alone
(2) Target+BiTE—target cells with 200 ng/mL of BiTE
(3) WU76UB—target cells co-cultured with WU76UB-NK cells at a 1:1 effector to target ratio
(4) WU76UB+BiTE—target cells co-cultured with WU76UB-NK cells at a 1:1 effector to target ratio in the presence of 200 ng/mL of BiTE
   The WU76UB-NK cells effectively killed NALM6 target cells (FIG. 20A) and HT144 target cells (FIG. 20B) in the presence of Blinatumomab and EGFR BiTE, respectively. These results show that the intracellular domains of the CD3e chains in the constructs used to transduce NK cells can vary and BiTE specific tumor cell killing by transduced NK cells is maintained.

### Example 6: NK Cells Expressing CD3e Chimeric Receptors Display CD3-Specific Signal Transduction and NFAT Signaling is Induced Downstream of Various CD3e Chimeric Receptors

Untransduced NK cells, WU76UB-NK cells and WU76Z-NK cells were stimulated with NALM6 target cells in the presence of 200 ng/mL of Blinatumomab (NALM+BiTE), PMA/Ionomycin (PMA:J, as a positive control), or left unstimulated (Unstim). Cells are then fixed, permeabilized, and stained with a fluorophore conjugated antibody against phosphorylated S6 protein. Phospho-S6 levels were measured by flow cytometry. As shown in FIG. 21A, there was robust p-S6 signaling in WU76UB-NK cells in the presence of Blinatumomab and NALM6 tumor target cells. As shown in FIG. 21B, the fold change in p-S6 levels was about 3 fold in WU76UB-NK cells and about 2 fold in WU76Z-NK cells in the presence of Blinatumomab and NALM6 target cells compared to NALM6 target cells alone. These results demonstrate that there is CD3-specific signal transduction in the engineered NK cells in the presence of a T cell engager and a tumor target.

Jurkat NFAT-GFP reporter cells were then used to test expression and downstream signaling of various CD3 encoding sequences. Jurkat reporter cells were engineered using CRISPR/Cas9 to knock out the T cell receptor alpha chain (TRAC). The knockout Jurkat reporter cells were then transduced with the WU76B, WU76E, WU76UB, and WU76Z constructs using lentiviral vectors. The expression of CD3 on the different Jurkat reporter cells are shown in FIG. 22A. The different populations of engineered Jurkat cells were challenged with SKOV3 tumor cells in the absence (SKOV3) or presence (SKOV3+EGFR BiTE) of an EGFR targeting T cell engager. As shown in FIG. 22B, WT Jurkat cells demonstrated a strong response upon addition of the EGFR T cell engager, compared to the TRAC^{-/-} cells, where there was no response. While the signaling null construct, WU76B, recapitulated the TRAC^{-/-} response, the other CD3 constructs demonstrated expression of GFP in response to the presence of the EGFR T cell engager. These results indicate that the CD3 constructs are inducing signaling through NFAT in the engineered NK cells.

### Example 7: NK Cells Expressing CD3e Chimeric Receptors Display Antitumor Immune Properties

IFNγ plays an important role in the activation of cellular immunity and contributes to an antitumor immune response. Experiments were performed to assess the capacity of engineered NK cells expressing CD3e chimeric receptors to produce IFNγ upon recognition of target cancer cells in the presence of T cell engagers.

Untransduced NK cells (NTD), WU76UB-NK cells and WU76Z-NK cells were stimulated with PMA+Ionomycin, NALM6 target cells in the presence of 200 ng/mL of Blinatumomab (E+T+BiTE), NALM6 target cells alone (E+T), or left unstimulated (E Only). Cells were then fixed, permeabilized, and stained with a fluorophore conjugated antibody against interferon gamma (IFNγ). Intracellular IFNγ levels were measured by flow cytometry. While untransduced NK cells only produced background levels of IFNγ in the presence of target cells and Blinatumomab, WU76B-NK cells (FIG. 23A and FIG. 23B) and WU76Z-NK cells (FIG. 23B) produced increased amounts of IFNγ when stimulated with target cells in the presence of Blinatumomab, showing their potential in enhancing the antitumor immune response.

As shown in the preceding examples, NK cells expressing the CD3e chimeric receptors are potent cytotoxic cells. Granzyme B is an apoptotic protease found in the lytic granules of NK cells. Upon activation, NK cells release granzyme B to kill tumor cells. Untransduced NK cells (NTD) or WU76UB-NK cells were cocultured with NALM6 target cells and Blinatumomab (Target+BiTE) or NALM6 cells only (Target Only). Supernatants from the cocultures were harvested after 24 hours and levels of secreted granzyme B were measured by ELISA. As shown in FIG. 24, in cocultures with NALM6 targets, WU76UB-NK cells released significantly higher levels of granzyme B compared to untransduced NK cells in the presence of Blinatumomab. These results further demonstrate that the CD3 chimeric receptors confer the engineered NK cells potent, antigen specific, cytotoxic activity.

### Example 8: WU76UB Restores CD3 Expression in TRAC^{-/-} T Cells

Experiments were performed to assess CD3 cell surface expression in TRAC^{-/-} (TRAC^{KO}) T cells transduced with the WU76UB construct. T cells were harvested from human PBMCs via magnetic separation. Isolated T cells were then activated with anti-CD28 and anti-CD3 antibodies-coated beads and cultured in TexMACS media containing 3% human AB serum (HAB) and 10 ng/mL IL-7 and IL-15. Two days after activation, T cells were electroporated with CRISPR/CAS9 protein complexed to TRAC sgRNA to delete expression of TCRαβ and CD3. T cells were then transduced with lentivirus encoding WU76UB by electroporation. Five days after viral transduction, cells were collected and analyzed for TCRαβ and CD3 cell surface expression by flow cytometry. As shown in FIG. 25, in TRAC^{KO} T cells, deletion of TRAC significantly diminished expression of the T cell receptor (TCRαβ) and CD3 compared to control T cells. Importantly, CD3 expression was restored in TRAC^{KO} T cells transduced with WU76UB in the absence of TCR. These results demonstrate that CD3e chimeric receptors were stably expressed by engineered TRAC^{KO} T cells, allowing engineered T cells that lack native T cell receptor expression to bind to T cell engagers.

1. A chimeric receptor capable of being expressed in a natural killer (NK) cell, wherein the chimeric receptor comprises:
a CD3 epsilon (CD3e) extracellular domain;
a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

2. A chimeric receptor capable of being expressed in a natural killer (NK) cell, wherein the chimeric receptor comprises:
a CD3 epsilon (CD3e) extracellular domain;
a first transmembrane domain selected from the group consisting of: CD3d, CD3e, CD3g, CD16, NKG2D, DAP10, FcγRIIIa, NKp44, NKp30, NKp46, actKIR, NKG2C, CD8α, and IL15; and
a first intracellular signaling domain selected from the group consisting of: CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

3. A bicistronic chimeric receptor comprising a first and a second chimeric receptor, wherein the first and the second chimeric receptors are co-expressed by a bicistronic vector, wherein the first chimeric receptor comprises a CD3e extracellular domain; and wherein the second chimeric receptor comprises a CD3g or a CD3d extracellular domain.

4. The bicistronic chimeric receptor of claim 3, wherein the first chimeric receptor comprises:
(a) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

5. The bicistronic chimeric receptor of claim 3, wherein the first chimeric receptor comprises:
(a) a first transmembrane domain selected from the group consisting of: CD3e, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3d, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

6. The bicistronic chimeric receptor of claim 3, wherein the first chimeric receptor comprises a CD3e extracellular domain, a CD3e transmembrane domain, and a CD3zeta intracellular domain; and wherein the second chimeric receptor comprises a CD3g extracellular domain, a CD3g transmembrane domain, and a 41BB intracellular domain.

7. The bicistronic chimeric receptor of claim 3, wherein the first chimeric receptor comprises a CD3e extracellular domain, a CD3e transmembrane domain, and CD3zeta and 2B4 intracellular domains; and wherein the second chimeric receptor comprises a CD3g extracellular domain, a CD3g transmembrane domain, and a OX40 intracellular domain.

8. A tricistronic chimeric receptor comprising a first, a second, and a third chimeric receptor, wherein the first, the second, and the third chimeric receptors are co-expressed by a tricistronic vector, wherein the first chimeric receptor comprises a CD3e extracellular domain; wherein the second chimeric receptor comprises a CD3g extracellular domain; and wherein the third chimeric receptor comprises a CD3d extracellular domain.

9. The tricistronic chimeric receptor of claim 8, wherein the first chimeric receptor comprises:
(a) a first transmembrane domain selected from the group consisting of: CD3d, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3e, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

10. The tricistronic chimeric receptor of claim 8, wherein the first chimeric receptor comprises:
(a) a first transmembrane domain selected from the group consisting of: CD3e, CD3g, CD16, NKG2D, DAP10, FcγRIIIa, NKp44, NKp30, NKp46, actKIR, NKG2C, CD8α, and IL15; and
(b) a first intracellular signaling domain selected from the group consisting of: CD3d, CD3g, 2B4, CD79A, CD79B, CD132, IL2R beta, 4-1BB, FcR gamma ITAM, CD3zeta ITAM, DNAM-1, NKp80, NTBA, CRACC, CD2, CD27, integrins, IL-15R, IL-18R, IL-12R, IL-21R, IRE1a, and OX40.

11. The tricistronic chimeric receptor of claim 8, wherein the first chimeric receptor comprises a CD3e extracellular domain, a CD3e transmembrane domain, and a CD3z intracellular domain; wherein the second chimeric receptor comprises a CD3d extracellular domain, a CD3d transmembrane domain, and a 2B4 intracellular domain; and wherein the third chimeric receptor comprises a CD3g extracellular domain, a CD3g transmembrane domain, and a 4-1BB intracellular domain.

12. A vector comprising a nucleic acid encoding the bicistronic chimeric receptor of claim 3.

13. An engineered NK cell comprising
the bicistronic chimeric receptor of claim 3.

14. The engineered NK cell of claim 13, wherein the engineered NK cell is deficient for NKG2A and/or CD8 expression, activity, or signaling.

15. The engineered NK cell of claim 13, wherein the extracellular domain of the bicistronic chimeric receptor is capable of binding to a T cell engager.

16. An engineered T cell comprising
the bicistronic chimeric receptor of claim 3.

17. The engineered T cell of claim 16, wherein the T Cell Receptor Alpha chain (TRAC) gene is genetically modified or deleted.

18. The engineered T cell of claim 16, wherein the extracellular domain is capable of binding to a T cell engager.

19. The engineered NK cell of claim 15, wherein the T cell engager is selected from the list consisting of a bispecific T cell engager (BiTE), a trispecific T cell engager (TriTE), a TeTriTE, and a dual affinity retargeting antibody (DART).

20. A method of inducing an immune response to a disease in a subject in need thereof comprising administering to the subject the engineered NK cell of claim 13.

21. The method of claim 20, comprising administering to the subject a T cell engager.
